(19) United States
(12) Patent Application Publication
(10) Pub. No.: US 2019/0351017 A1
(43) Pub. Date: Nov. 21, 2019

(54) METHODS AND COMPOSITIONS FOR TREATING HYPOGLYCEMIA

(71) Applicant: Joslin Diabetes Center, Inc., Boston, MA (US)

(72) Inventors: Mary-Elizabeth Patti, Newton, MA (US); Allison Goldfine, Wayland, MA (US)

(21) Appl. No.: 16/265,290

(22) Filed: Feb. 1, 2019

Related U.S. Application Data
(63) Continuation of application No. PCT/US2017/045061, filed on Aug. 2, 2017.
(60) Provisional application No. 62/370,532, filed on Aug. 3, 2016.

(51) Int. Cl. A61K 38/18; A61P 3/08

(52) U.S. Cl. CPC A61K 38/1825; A61K 38/30; A61P 3/08

(57) ABSTRACT

The invention provides methods and compositions relating to molecular targets associated with treating or preventing hypoglycemia. Included in the invention are methods and compositions relating to inhibiting the expression or activity of a glucose modulating agent associated with hypoglycemia e.g., Fibroblast Growth Factor 19 (FGF19). Also included in the invention are methods and compositions for increasing the blood glucose level of a subject. Additional aspects of the invention relate to methods for determining whether a subject has or is at risk for developing hypoglycemia, for example, post-bariatric hypoglycemia.

### RELATED APPLICATIONS

This application is a Continuation of International Application No. PCT/US2017/045061, filed Aug. 2, 2017, which claims priority to U.S. Provisional Patent Application No. 62/370,532 filed Aug. 3, 2016, and entitled “Methods and Compositions for Treating Hypoglycemia.” Each of the foregoing applications is incorporated herein by reference in its entirety.

### SEQUENCE LISTING

The instant application contains a Sequence Listing which has been submitted electronically in ASCII format and is hereby incorporated by reference herein in its entirety. Said ASCII copy, created on Feb. 1, 2019, is named J103021_1030US.PCT_Sequence_Listing.txt and is 269 kilobytes in size.

### BACKGROUND OF THE INVENTION

Obesity and related comorbidities, such as type 2 diabetes and cardiovascular disease, are increasingly recognized as a major threat to individual and public health. Unfortunately, it is very difficult to achieve sustained weight loss with current medical approaches. Given these critical unmet needs, both clinicians and patients alike have embraced the results of recent controlled clinical trials demonstrating potent effects of bariatric surgical procedures to not only induce sustained weight loss but also to improve or normalize obesity-related comorbidities, including type 2 diabetes (Sjostrom, L. J. Intern. Med. 273, 219-234, 2013; Schauer, P. R. et al. N. Engl. J. Med. 366, 1567-1576, 2012; Mingrone, G. et al. N. Engl. J. Med. 2012; Schauer, P. R. et al. N Engl. J Med, 2014; Zaloga, G. P. & Dons, R. F. Dig. Dis Sci 29, 1164-1166, 1984). Remarkably, surgery is superior to medical therapy for weight loss and diabetes, improves lifespan, and results in sustained improvement in glycemic control and reduced need for medications (Schauer, P. R. et al. N Engl. J Med, 2014). Such data have led to an explosion in the number of bariatric surgeries performed in the US—an estimated 179,000 in 2013 (Estimate of Bariatric Surgery Numbers, 2014. ASMB). While benefits of bariatric surgery are achieved with low operative mortality (Sjostrom, L. J. Intern. Med. 273, 219-234, 2013), longer-term intestinal and nutritional complications can occur.

One particularly challenging and sometimes severe complication of bariatric surgery is hyperinsulinemic hypoglycemia (Service, G. J. et al. N Engl J Med 353, 249-254, 2005; Patti, M. E. et al. Diabetologia 48, 2236-2240, 2005). While most commonly associated with roux-en-Y gastric bypass, hypoglycemia has also been observed following sleeve gastrectomy (Papamargaritis, D. et al. Obes. Surg. 22, 1600-1606, 2012), but is rarely reported after banding (Scavini, M., et al. N Engl J Med 353, 2822-2823, 2005), and is qualitatively similar to hypoglycemia reported after gastrostomy for ulcers or fundoplication in children and adults (Palladino, A. A. et al. J Clin Endocrinol Metab 94, 39-44, 2009; Ng, D. D. et al. J Pediatr 139, 877-879, 2001; Bernard, B. et al, BMC. Gastroenterol. 10, 77, 2010).

Post-bariatric hypoglycemia (PBH) typically occurs within 1-3 hours after meals, and is not present after prolonged fasting. Plasma insulin concentrations are inappropriately high at the time of hypoglycemia, indicating dysregulation of insulin secretion as an important mechanism (Goldfine, A. B. et al. J. Clin. Endocrinol. Metab 92, 4678-4685, 2007). Mild, often undiagnosed, hypoglycemia is increasingly recognized as a potential contributor to increased appetite and weight regain (Roslin, M. et al. Surg. Endosc. 25, 1926-1932, 2011). More severely affected patients can develop profound neuroglycopenia, with loss of consciousness, seizures and motor vehicle accidents.

Current therapy for hypoglycemia is focused on diet and use of specific medications. Unfortunately, these are not typically adequate. Dietary modification is aimed at reducing intake of high glycemic index carbohydrates (Kellogg, T. A. et al. Surg. Obes. Relat Dis. 4, 492-499, 2008). Both diet and pre-meal acarbose (Valderas, J. P. et al. Obes. Surg. 22, 582-586, 2012) aim to minimize rapid postprandial surges in glucose which are triggers for glucose-dependent insulin secretion. Continuous glucose monitoring can be helpful to improve patient safety, particularly for those with hypoglycemic unawareness (Halperin, F., et al, J. Obes. 2011, 869536). Additional therapies include octreotide (to reduce incretin and insulin secretion) (Myint, K. S. et al. Eur. J. Endocrinol. 166, 951-955, 2012), diazoxide (to reduce insulin secretion) (Spanakis, E. & Gragnoli, C. Obes. Surg. 19, 1333-1334, 2009), calcium channel blockade (to reduce insulin secretion) (Moreira, R. O., et al, Obes. Surg. 18, 1618-1621, 2008), gastric restriction or banding (to slow gastric emptying) (Fernandez-Esparrach, G., et al, Surg. Obes. Relat Dis. 6, 36-40, 2010), and providing nutrition solely through a gastrostomy tube placed into the bypassed duodenum (Fernandez-Esparrach, G., et al, Surg. Obes. Relat Dis. 6, 36-40, 2010; McLaughlin, T., et al, J Clin Endocrinol Metab 95, 1851-1855, 2010). Pancreatic resection was initially employed for patients with life-threatening hypoglycemia; however, this procedure is not uniformly successful in remitting hypoglycemia and thus is not routinely recommended at the present time. Surprisingly, reversal of gastric surgery is not uniformly successful (Patti, M. E. et al. Diabetologia 48, 2236-2240, 2005; Lee, C. J. et al. J Clin Endocrinol Metab 98, E1208-E1212, 2013), suggesting the importance of underlying genetics and/or compensatory mechanisms which persist after surgical reversal.

Despite strict adherence to medical nutrition therapy and clinical use of multiple medical options above, usually in combination, many patients continue to have frequent hypoglycemia. While hypoglycemia most commonly occurs in the postprandial state, it can also be observed in response to increased activity and emotional stress. Patient safety is additionally compromised when hypoglycemia unawareness develops with recurrent hypoglycemia. Patients are often disabled by hypoglycemia which occurs multiple times per day, leading to inability to drive or maintain employment, and causing fear of eating and exercise due to potential provocation of hypoglycemic events, cardiac arrhythmias (Clark, A. L., et al, Diabetes 63, 1457-1459, 2014), syncope, falls, and seizures. Thus, there is an urgent need for new approaches to the treatment of severe hypoglycemia to maintain health, allow optimal nutrition, and improve safety.

### SUMMARY OF THE INVENTION

The present invention is based, at least in part, on the discovery that certain molecular targets are mediators of hypoglycemia. Modulating the activity or expression of these targets can alter the blood glucose level of a subject and serve to treat or prevent hypoglycemia, including, for example, hypoglycemia in a subject having or at risk for PBH. The methods and compositions of the invention are based on the identification of proteins associated with hypoglycemia, including post-bariatric hypoglycemia (PBH). These proteins are described throughout as glucose modulating molecules, as these molecules are either overexpressed or underexpressed in PBH patients, relative to patients who do not have hypoglycemia.

Accordingly, in one aspect, the invention provides a method of increasing the blood glucose level of a subject in need thereof, comprising administering an antagonist of a glucose modulating molecule to the subject, such that the blood glucose level of the subject is increased, wherein the glucose modulating molecule is FGF19, IGFBP1, ADIPOQ, GCG, SHBG, CXCL3, CXCL2, TNFRSF17, AMICA1, TFF3, EFNB3 and/or LSAMP, or combinations thereof.

In another aspect, the invention provides a method of treating or preventing hypoglycemia in a subject in need thereof, comprising administering an antagonist of a glucose modulating molecule to the subject, such that hypoglycemia is treated or prevented, wherein the glucose modulating molecule is FGF19, IGFBP1, ADIPOQ, GCG, SHBG, CXCL3, CXCL2, TNFRSF17, AMICA1, TFF3, EFNB3 and/or LSAMP, or combinations thereof.

In one embodiment of the foregoing aspects, the subject has undergone bariatric surgery. In some embodiments, the bariatric surgery is gastric bypass, roux-en-Y gastric bypass, biliopancreatic bypass, duodenal switch, gastric banding, gastrectomy, sleeve gastrectomy, fundoplication, and/or other gastrointestinal surgical procedures. In another embodiment, the subject has reactive hypoglycemia.

In some embodiments, the antagonist of the glucose modulating molecule can be an antibody, or an antigen binding fragment thereof, which specifically binds the glucose modulating molecule. In other embodiments, the antagonist can be a soluble form of a receptor specific for the glucose modulating molecule. In some embodiments, the antagonist can be a small molecule inhibitor specific for the glucose modulating molecule. In other embodiments, the antagonist can be an antisense oligonucleotide specific for the glucose modulating molecule. In other embodiments, the antagonist can be an inhibitory aptamer that specifically binds the glucose modulating molecule.

In certain embodiments, the glucose modulating molecule is FGF19. In some embodiments, the antagonist is an FGF19 inhibitor.

In some embodiments, the glucose modulating molecule is FGF19, and the antagonist of FGF19 is an inhibitor of an FGF19 receptor, e.g., FGFR4 or Klotho. In exemplary embodiments, the inhibitor of the FGF19 receptor is selected from the group consisting of an anti-FGFR4 antibody, or an antigen binding fragment thereof, a small molecule inhibitor specific for FGFR4, an antisense oligonucleotide specific for FGFR4, an aptamer that specifically binds FGFR4, an anti-Klotho antibody, or an antigen binding fragment thereof, a small molecule inhibitor specific for Klotho, an antisense oligonucleotide specific for Klotho, and an aptamer that specifically binds Klotho.

In another aspect, the invention provides a method of increasing the blood glucose level of a subject in need thereof, comprising administering an agonist of a glucose modulating molecule to the subject, wherein the glucose modulating molecule is HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and/or SORCS2, or combinations thereof, such that the blood glucose level of the subject is increased.

In another aspect, the invention provides a method of treating or preventing hypoglycemia in a subject in need thereof, comprising administering an agonist of a glucose modulating molecule to the subject, such that hypoglycemia is treated or prevented, wherein the glucose modulating molecule is HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and/or SORCS2, or combinations thereof.

In one embodiment of the foregoing aspects, the subject has undergone bariatric surgery. In some embodiments, the bariatric surgery is gastric bypass, roux-en-Y gastric bypass, biliopancreatic bypass, duodenal switch, gastric banding, gastrectomy, sleeve gastrectomy, fundoplication, and/or other gastrointestinal surgical procedures. In another embodiment, the subject has reactive hypoglycemia.

In some embodiments, the agonist of the glucose modulating molecule is an agonist antibody, or an antigen binding fragment thereof, which specifically binds the glucose modulating molecule, or a receptor thereof. In other embodiments, the agonist is a small molecule specific for the glucose modulating molecule. In some embodiments, the agonist is a stimulatory aptamer that specifically binds the glucose modulating molecule.

In some embodiments, the agonist of the glucose modulating molecule is a protein having an amino acid sequence of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB or SORCS2, or a nucleic acid encoding HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB or SORCS2.

In another aspect, the invention provides a method of determining whether a subject has or is at risk for having post-bariatric hypoglycemia (PBH), comprising determining the level of a glucose modulating molecule(s) in a sample obtained from the subject, wherein the glucose modulating molecule is FGF19, IGFBP1, ADIPOQ, GCG, SHBG, CXCL3, CXCL2, TNFRSF17, AMICA1, TFF3, EFNB3 and/or LSAMP, or a combination thereof; and comparing the level of the glucose modulating molecule(s) in the sample to a control level of the glucose modulating molecule from a subject who does not have or is not at risk for having PBH; wherein an increase in the level of the glucose modulating molecule(s) in the sample relative to the control level is indicative that the subject has or is at risk for post-bariatric hypoglycemia; and wherein no change or a decrease in the level of the glucose modulating molecule in the sample relative to the control is indicative that the subject does not have or is not at risk for post-bariatric hypoglycemia.

In another aspect, the invention provides a method of determining whether a subject has or is at risk for having post-bariatric hypoglycemia (PBH), comprising determining the level of a glucose modulating molecule(s) in a sample obtained from the subject, wherein the glucose modulating molecule is HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and/or SORCS2, or a combination thereof; and comparing the level of the glucose modulating molecule(s) in the sample to a control level of the glucose modulating molecule from a subject who does not have or is not at risk for having PBH; wherein decrease in the level of the glucose modulating molecule(s) in the sample relative to the control level is indicative that the subject has or is at risk for post-bariatric hypoglycemia; and wherein no change or an increase in the level of the glucose modulating molecule in the sample relative to the control is indicative that the subject does not have or is not at risk for post-bariatric hypoglycemia.

In one embodiment of the foregoing aspects, the sample is a blood sample. In another embodiment, the sample is a plasma sample. In another embodiment, the sample is a serum sample.

In another embodiment of the foregoing aspects, the method further comprises administering a therapeutically effective amount of an antagonist of a glucose modulating molecule to the subject, wherein the glucose modulating molecule is selected from a group consisting of FGF19, IGFBP1, ADIPOQ, GCG, SHBG, CXCL3, CXCL2, TNFRSF17, AMICA1, TFF3, EFNB3 and/or LSAMP. In another embodiment, the method further comprises administering a therapeutically effective amount of an agonist of a glucose modulating molecule to the subject, wherein the glucose modulating molecule is selected from a group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and/or SORCS2.

In another aspect, the invention provides a method of selecting a bariatric surgery for a subject having obesity, comprising comparing the level of one or more glucose modulating molecule(s) selected from the group consisting of FGF19, IGFBP1, ADIPOQ, GCG, SHBG, CXCL3, CXCL2, TNFRSF17, AMICA1, TFF3, EFNB3, LSAMP, and combinations thereof, in a sample obtained from the subject to a control level of the glucose modulating molecule in a comparable sample from a subject who does not have or is not at risk for post-bariatric hypoglycemia (PBH), and selecting a bariatric surgery for the subject if the level of the one or more glucose modulating molecule(s) in the sample obtained from the subject is equivalent to or lower than the control level of the one or more glucose modulating molecules.

In one embodiment of this aspect, a treatment other than bariatric surgery is selected for a subject having obesity if the level of the one or more glucose modulating molecule(s) in the sample obtained from the subject is higher than the control level of the one or more glucose modulating molecules.

In another aspect, the invention provides a method of selecting a bariatric surgery for a subject having obesity, comprising comparing the level of one or more glucose modulating molecule(s) selected from the group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB, SORCS2, and combinations thereof, in a sample obtained from the subject to a control level of the glucose modulating molecule in a comparable sample from a subject who does not have or is not at risk for post-bariatric hypoglycemia (PBH), and selecting a bariatric surgery for the subject if the level of the one or more glucose modulating molecule(s) in the sample obtained from the subject is equivalent to or higher than the control level of the one or more glucose modulating molecules.

In one embodiment of this aspect, a treatment other than bariatric surgery is selected for a subject having obesity if the level of the one or more glucose modulating molecule(s) in the sample obtained from the subject is lower than the control level of the one or more glucose modulating molecules.

In one embodiment of the foregoing aspects, the method can further comprise determining the level of the one or more glucose modulating molecule(s) in a sample obtained from the subject. In exemplary embodiments, the sample is a blood sample, e.g., a plasma sample or a serum sample.

### BRIEF DESCRIPTION OF THE DRAWINGS

FIG. 1 depicts an exemplary pattern of continuous glucose monitoring (CGM) tracing in a patient with post bariatric hypoglycemia (PBH) in the ambulatory state. Food intake and rapid emptying of the gastric pouch triggers a brisk and excessive rise in glucose (1st arrow), with subsequent rapid decline in glucose precipitating adrenergic symptoms (2nd arrow). Despite treatment with glucose tablets, the patient subsequently developed more severe hypoglycemia (51 mg/dl) with neuroglycopenic symptoms (3rd arrow).

FIG. 2 graphically depicts multiple interacting pathways that may contribute to PBH: 1) increased gastric emptying, 2) increased intestinal secretion of metabolically active hormones, such as GLP1, incretins, and FGF19, 3) intestinal mucosal adaptations, 4) disordered pancreatic islet function with increased insulin secretion and β-cell glucose responsiveness, 5) altered bile acid composition or content, 6) gut microbiota, 7) altered hepatic glucose uptake and metabolism, or counterregulatory responses.

FIG. 3 graphically depicts the postprandial plasma levels of FGF19 protein (described as RFU) in patients with PBH, and asymptomatic post-surgical patients, as determined using the Somalogic platform.

FIG. 4 graphically depicts the postprandial plasma levels (pg/ml) of FGF19 protein in patients with PBH and asymptomatic post-surgical patients as determined by ELISA.

FIG. 5A provides a table describing proteins that were determined to have increased expression levels in patients with PBH, and FIG. 5B provides a table describing proteins determined to have decreased expression levels in patients with PBH. The molecular targets described in FIGS. 5A and 5B may contribute to insulin-independent metabolic changes and may serve as novel therapeutic targets for improving hypoglycemia in patients.

### DETAILED DESCRIPTION OF THE INVENTION

### I. Definitions

In order that the present invention may be more readily understood, certain term are first defined.

As used herein, the term “hypoglycemia” refers to a condition characterized by abnormally low blood glucose (blood sugar) levels. In one embodiment, a subject having hypoglycemia has a blood sugar level which is less than about 70 mg/dl.

As used herein, the term “glucose modulating molecule” refers to a gene or a protein whose activity (directly or indirectly) is capable of modulating, e.g., increasing or decreasing, the level of glucose in a subject, e.g., a human subject. In one embodiment, the glucose modulating molecule is able to modulate glucose levels in the blood of a human subject. In one embodiment, the glucose modulating molecule is a protein. An example of a glucose modulating molecules whose expression and/or activity levels are negatively correlated with the level of glucose in a subject includes, but is not limited to, FGF19, IGFBP1, ADIPOQ, GCG, SHBG, CXCL3, CXCL2, TNFRSF17, AMICA1, TFF3, EFNB3, LSAMP. An example of a glucose modulating agent whose expression and/or activity levels are positively correlated with the level of glucose in a subject includes, but is not limited to, HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2.

As used herein, the terms,“inhibitor of a glucose modulating molecule,” and “antagonist of a glucose modulating molecule,” refer to an agent that partially or fully blocks, inhibits, or neutralizes a biological activity mediated by a glucose modulating molecule.

As used herein, the terms, “activator of a glucose modulating molecule,” and “agonist of a glucose modulating molecule,” refer to an agent that partially or fully activates, stimulates, or increases a biological activity mediated by a glucose modulating molecule.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH 1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from aminoterminus to carboxy-terminus in the following order: FR1, CDR1, FR1, CDR2, FR3, CDR3, FR4.

The term “antigen-binding portion” or “antigen-binding fragment” of an antibody (or simply “antibody portion”), as used herein, refers to a portion of a full-length antibody, generally the target binding or variable region. Examples of antibody fragments include Fab, Fab′, F(ab′)₂ and Fv fragments. The phrase “functional fragment” of an antibody is a compound having qualitative biological activity in common with a full-length antibody. For example, a functional fragment of an FGF19 antibody is one which can bind to FGF19 in such a manner so as to block, inhibit, or neutralize a biological activity mediated by FGF19. As used herein, “functional fragment” with respect to antibodies, refers to Fv, scFv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. An scFv contains one heavy and one light chain variable domain connected by a linker peptide of a size that permits the VH and VL domains to interact to form the target binding site. Collectively, the six CDRs confer target binding specificity to the antibody or antibody fragment. However, even a single variable domain (or half of an Fv comprising only three CDRs specific for a target) can have the ability to recognize and bind target, although at a lower affinity than the entire binding site.

The terms “antagonist antibody” or “blocking antibody” as used herein refer to an antibody which inhibits or reduces the biological activity of the antigen to which it binds. Exemplary antagonist antibodies substantially or completely inhibit the biological activity of the antigen.

The terms “agonist antibody” or “activating antibody” as used herein refer to an antibody which increases or activates the biological activity of the antigen to which it binds. Exemplary agonist antibodies substantially or completely increase the biological activity of the antigen.

The term “subject,” as used herein, refers to either a human or non-human animal. In one embodiment, the subject is a human subject. In another embodiment, the subject is a mammal.

The term “detection” includes any means of detecting, including direct and indirect detection.

The “presence,” “amount” or “level” refers to a detectable level of a protein or nucleic acid in a biological sample. A level may be measured by methods known to one skilled in the art and also disclosed herein.

The terms “express,” “expression,” or “expressed”, used interchangeably herein, refer to a gene that is transcribed or translated at a detectable level. Unless otherwise specified, expression refers either protein or RNA levels.

“Increased expression,” “elevated expression,” “elevated expression levels,” or “elevated levels” refers to an increased expression or increased levels of a certain nucleic acid(s) or protein(s) in an individual relative to a suitable control, such as an individual or individuals who are not suffering from a disease or disorder (e.g., hypoglycemia) or an internal control (e.g., housekeeping biomarker). In some embodiments, a suitable control can be a known standard value or range of values representative of a “normal” subject, i.e., a subject not afflicted with hypoglycemia.

“Decreased expression,” “reduced expression,” “reduced expression levels,” or “reduced levels” refers to a decrease expression or decreased levels of a certain nucleic acid(s) or protein(s) in an individual relative to a control, such as an individual or individuals who are not suffering from the disease or disorder (e.g., hypoglycemia) or an internal control (e.g., housekeeping biomarker).

The terms “sample,” or “biological sample” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

A “therapeutically effective amount” of a therapeutic agent, or combinations thereof, is an amount sufficient to treat disease in a subject. For example, a therapeutically effective amount of an FGF19 antagonist can be an amount of an agent that provides an observable therapeutic benefit compared to baseline clinically observable signs and symptoms of hypoglycemia, e.g., by increasing blood glucose levels.

The term “about” or “approximately” generally means within 5%. In one embodiment, the term about refers to a number(s) which is within 1%, of a given value or range.

As used herein, the term “isolated” refers to a molecule, e.g., a protein or nucleic acid, which is separated from other molecules that are present in the natural source of the molecule. In one embodiment, an “isolated” molecule is substantially free of other cellular material, or culture media when produced by recombinant techniques, or, in the alternative, substantially free of chemical precursors or other chemicals when chemically synthesized. A molecule that is substantially free of cellular material includes preparations having less than about 30%, 20%, 19%, 18%, 17%, 16%, 15%, 14%, 13%, 12%, 11%, 10%, 9%, 8%, 7%, 6%, or about 5% of heterologous molecules and which retains the biological activity of the molecule.

As used herein, the term “vector” refers to a nucleic acid molecule capable of transporting another nucleic acid to which it has been linked.

As used herein, the term “mimetic” when made in reference to a protein refers to a molecular structure which serves as a substitute for a protein used in the present invention (see Morgan et al. (1989) Ann. Reports Med. Chem. 24:243-252 for a review of peptide mimetics). In one embodiment, a mimetic may be an organic compound that imitates the binding site of a specific FGF protein, and, therefore, the functionality of the FGF protein, e.g., increasing glucose levels in the blood of a hypoglycemic subject.

The term “isostere”, as used herein, is intended to include a chemical structure that can be substituted for a second. chemical structure because the steric conformation of the first structure fits a binding site specific for the second structure. The term specifically includes peptide backbone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets. Other examples of isosteres include peptides substituted with one or more benzodiazepine molecules (see e.g., James, G. L. et al. (1993) Science 260:1937-1942).

The term “subject” or “patient,” as used herein interchangeably, refers to either a human or non-human animal. In one embodiment, the subject is a human.

The term “dose,” as used herein, refers to an amount of an agent, (e.g., an FGF19 antagonist such as an anti-FGF19 antibody).

The term “dosing”, as used herein, refers to the administration of a substance (e.g., an FGF19 antagonist such as an anti-FGF19 antibody) to achieve a therapeutic objective (e.g., the treatment of hypoglycemia, including, but not limited to, PBH).

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

### II. Methods and Compositions of the Invention

Hypoglycemia is a condition characterized by abnormally low blood glucose (blood sugar) levels and may result in a variety of symptoms including clumsiness, trouble talking, confusion, loss of consciousness, seizures, or death. A feeling of hunger, sweating, shakiness, or weakness may also be present. The most common cause of hypoglycemia is medications used to treat diabetes mellitus such as insulin, sulfonylureas, and biguanides. (Yanai, H et al, World journal of diabetes 6 (1): 30-6, 2015). Other causes of hypoglycemia include kidney failure, certain tumors, liver disease, hypothyroidism, starvation, inborn error of metabolism, severe infections, reactive hypoglycemia, and a number of drugs including alcohol. (Schrier, Robert W. The internal medicine casebook real patients, real answers (3 ed.). Philadelphia: Lippincott Williams & Wilkins. p. 119. 2007).

Post-bariatric hypoglycemia (PBH) is defined as a plasma glucose level <70 mg/dl in conjunction with neuroglycopenia. Relief of PBH is normalization of glucose levels. Hypoglycemia typically occurs within 1-3 hours after meals, particularly meals rich in simple carbohydrates, and is not present after prolonged fasting. Plasma insulin concentrations are inappropriately high at the time of hypoglycemia, indicating dysregulation of insulin secretion as an important mechanism (Goldfine, A. B. et al. J. Clin. Endocrinol. Metab 92, 4678-4685, 2007). Hypoglycemic symptoms may be autonomic (e.g., palpitations, lightheadedness, sweating) or neuroglycopenic (e.g., confusion, decreased attentiveness, seizure, loss of consciousness). Early in the post-operative period, hypoglycemia is usually mild, often associated with dumping syndrome, and effectively treated with low glycemic index diets. Mild, often unrecognized, hypoglycemia is increasingly recognized as a potential contributor to increased appetite and weight regain (Roslin, M. et al. Surg. Endosc. 25, 1926-1932, 2011). A subset of post-bariatric patients develops very severe hypoglycemia with neuroglycopenia, with loss of consciousness, seizures and motor vehicle accidents, typically occurring 1-3 years following bypass. For these patients, a comprehensive multidisciplinary approach, including medical nutrition therapy and multiple medications, is required but often incompletely effective.

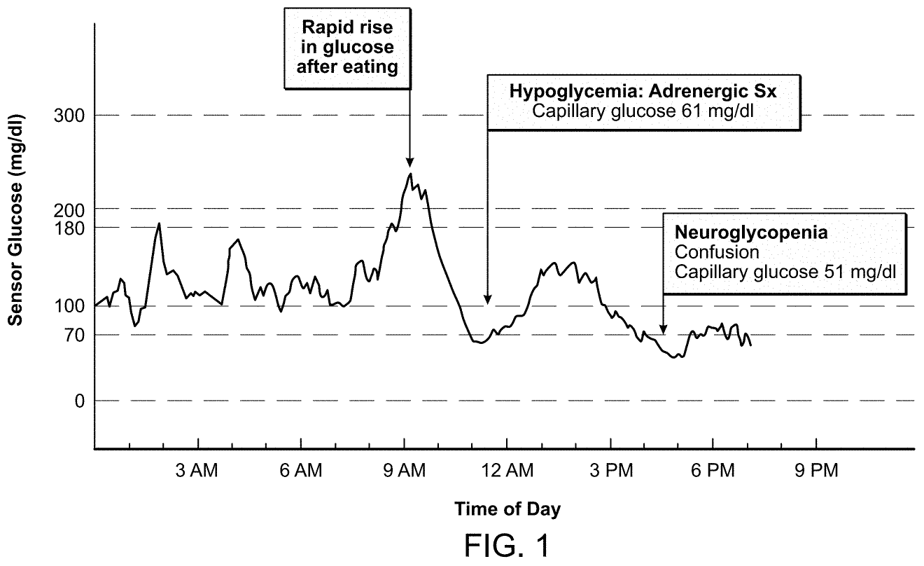

Metabolic studies in PBH patients reveal profound alterations in glycemic and hormonal patterns in the postprandial state occurring with gastric bypass anatomy and profound weight loss (Patti, M. E. & Goldfine, A. B. Gastroenterology 146, 605-608, 2014). A typical pattern in the ambulatory state, as revealed by continuous glucose monitoring (CGM), can be seen in FIG. 1. Food intake and rapid emptying of the gastric pouch triggers a brisk and excessive rise in glucose (1st red arrow), with subsequent rapid decline in glucose precipitating adrenergic symptoms (2nd red arrow). Despite treatment with glucose tablets, the patient subsequently developed more severe hypoglycemia (51 mg/dl) with neuroglycopenic symptoms (3rd red arrow). Clinical research studies from our group and others have demonstrated increased insulin secretion in the postprandial state in patients with severe PBH, as compared with asymptomatic post-GB or nonsurgical controls matched for degree of obesity (Service, G. J. et al. N Engl J Med 353, 249-254, 2005; Goldfine, A. B. et al. J. Clin. Endocrinol. Metab 92, 4678-4685, 2007; Salehi, M. et al, Diabetes 60, 2308-2314, 2011; Salehi, M., et al, Gastroenterology 146, 669-680, 2014). Although initial reports demonstrated pancreatic islet hypertrophy, pancreatic resection does not cure hypoglycemia (Patti, M. E. et al. Diabetologia 48, 2236-2240, 2005; Lee, C. J. et al. J Clin Endocrinol Metab 98, E1208-E1212, 2013), and excessive islet number has not been observed in all series (Service, G. J. et al. N Engl J Med 353, 249-254, 2005; Patti, M. E. et al. Diabetologia 48, 2236-2240, 2005; Meier, J. J., et al. Diabetes Care 29, 1554-1559, 2006; Reubi, J. C. et al. Diabetologia 53, 2641-2645, 2010). One candidate mediator of increased insulin secretion in PBH is GLP-1, an incretin peptide released from intestinal L-cells in response to meals, in turn stimulating insulin secretion in a glucose-dependent manner Indeed, postprandial levels of the incretin hormone GLP-1 are increased by >10-fold in post-bypass patients, are even higher in those with hypoglycemia, and correlate inversely with postprandial glucose levels (Goldfine, A. B. et al. J. Clin. Endocrinol. Metab 92, 4678-4685, 2007; Salehi, M., et al, Diabetes 60, 2308-2314, 2011). Furthermore, short-term pharmacologic blockade of the GLP-1 receptor markedly attenuates insulin secretion in post-bypass individuals, but increases GLP-1 levels in some studies (Salehi, M., et al, Gastroenterology 146, 669-680, 2014; Jorgensen, N. B. et al. Diabetes 62, 3044-3052, 2013). Interestingly, plasma levels of counterregulatory hormones such as cortisol and glucagon do not differ in patients with PBH vs. asymptomatic post-bypass patients during mixed meal testing (Goldfine, A. B. et al. J. Clin. Endocrinol. Metab 92, 4678-4685, 2007).

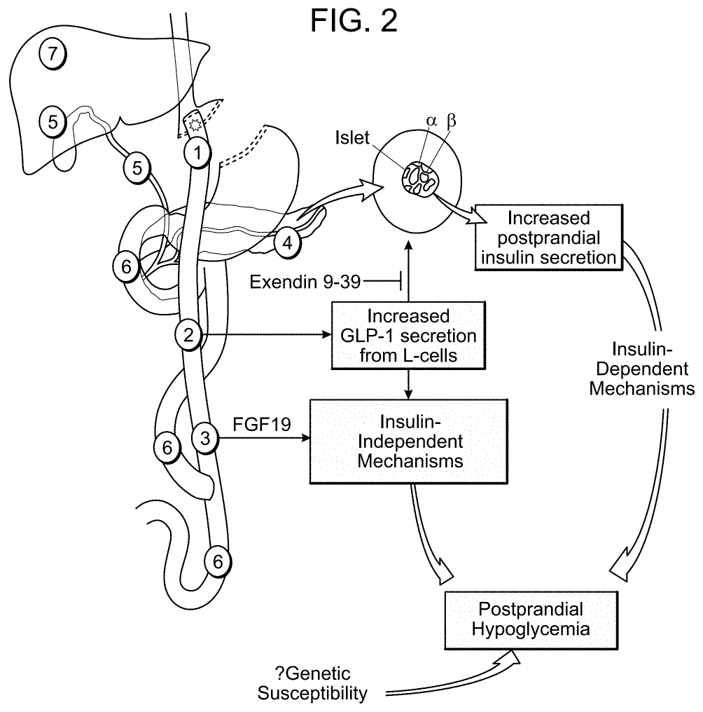

While increased insulin secretion is a central phenotype in PBH, recent studies have demonstrated additional insulin-independent factors. Insulin-independent glucose disposal is increased in patients with severe PBH (Patti, M. E.,et al, Obesity (Silver. Spring), 2015). Additional gastrointestinal factors which could modify systemic metabolism include dietary composition, gut microbiota (Liou, A. P. et al. Sci. Transl. Med. 5, 178ra41, 2013), bile acid composition (Patti, M. E. et al. Obesity (Silver. Spring). 2009), and intestinal adaptive responses (Hansen, C. F. et al. PLoS. ONE. 8, e65696, 2013). Collectively, these may influence absorption of glucose and other nutrients, intestinally-derived hormonal responses, and the magnitude of CNS-gut-liver regulatory loops. Finally, genetic variation could also contribute to altered hormonal responses and sensitivity (Mussig, K.,et al, Diabetologia 53, 2289-2297, 2010). Thus, while many interacting pathways contribute to PBH, as described in FIG. 2, the pathophysiology of PBH remains incompletely understood, limiting therapeutic options.

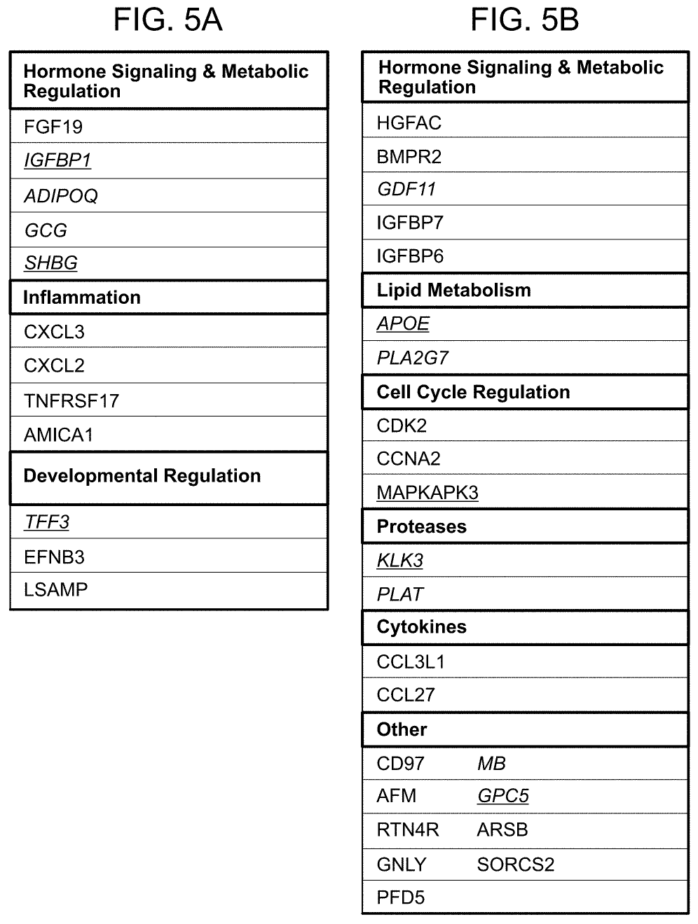

The present invention is based, at least in part, on the discovery that certain molecular targets are mediators of hypoglycemia. Modulating the activity or expression of these targets can alter the blood glucose level of a subject and serve to treat or prevent hypoglycemia, including, for example, hypoglycemia in a subject having or at risk for post-bariatric hypoglycemia (PBH). The methods and compositions of the invention are based on the identification of proteins associated with hypoglycemia, including PBH. These proteins are described throughout as glucose modulating molecules, as these molecules (see FIGS. 5A and 5B) are either overexpressed or underexpressed in PBH patients relative to patients who do not have hypoglycemia.

Accordingly, in one embodiment, the methods of the invention include methods of increasing the blood glucose level of a subject, by administering an inhibitor of FGF19, IGFBP1, ADIPOQ, GCG, SHBG, CXCL3, CXCL2, TNFRSF17, AMICA1, TFF3, EFNB3 or LSAMP, and/or an activator of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB or SORCS2. In other embodiments, the methods of the invention include treating or reducing the symptoms of hypoglycemia in a subject in need thereof, comprising administering an inhibitor of a FGF19, IGFBP1, ADIPOQ, GCG, SHBG, CXCL3, CXCL2, TNFRSF17, AMICA1, TFF3, EFNB3 or LSAMP, and/or an activator of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB or SORCS2.

FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2 are collectively (and individually) referred to herein as glucose modulator molecules given their differential expression in PBH patients.

### II.A. Glucose Modulating Molecules Whose Expression Levels are Upregulated in Subjects Having Hypoglycemia

One aspect of the present invention features a method for increasing the blood glucose level of a subject in need thereof by administering an agent that can decrease the expression or activity of a glucose modulating molecule whose protein levels are associated with hypoglycemia. In a one embodiment, the glucose modulating molecule is FGF19, IGFBP1, ADIPOQ, GCG, SHBG, CXCL3, CXCL2, TNFRSF17, AMICA1, TFF3, EFNB3 or LSAMP, or a combination thereof.

One aspect of the present invention features a method of increasing the blood glucose level of subject in need thereof, comprising administering an antagonist of one or more glucose modulating molecule(s) to the subject, such that the blood glucose level of the subject is increased, wherein the glucose modulating molecule is FGF19, IGFBP1, ADIPOQ, GCG, SHBG, CXCL3, CXCL2, TNFRSF17, AMICA1, TFF3, EFNB3 or LSAMP, or a combination thereof.

In another embodiment, the invention provides methods of treating or preventing hypoglycemia in a subject in need thereof, comprising administering an antagonist of one or more glucose modulating molecule(s) to the subject, wherein the glucose modulating molecule is FGF19, IGFBP1, ADIPOQ, GCG, SHBG, CXCL3, CXCL2, TNFRSF17, AMICA1, TFF3, EFNB3 or LSAMP, or a combination thereof, such that hypoglycemia is treated or prevented.

### 1. Glucose Modulating Molecules: Hormone Signaling and Metabolic Regulators

In one embodiment, the glucose modulating molecule is a hormone signaling or metabolic regulator. Inhibitors or antagonists of a hormone signaling or metabolic regulator may be used to increase the glucose level in a subject and may be used to treat or prevent hypoglycemia in a subject in need thereof. Examples of hormone signaling or metabolic regulators include FGF19, IGFBP1, ADIPOQ, GCG, and SHBG.

### FGF19

In one embodiment of the invention, an inhibitor of fibroblast growth factor 19 (FGF19) is used in the methods and compositions of the invention. The term “FGF19”, as used herein, refers to a native FGF19 from any vertebrate source, including mammals such as primates (e.g., humans), unless otherwise indicated. The term encompasses full-length, unprocessed FGF19, as well as any form of FGF19 that results from processing in a cell. The term also encompasses naturally occurring variants of FGF19, such as splice variants or allelic variants. The sequence of a human FGF19 mRNA sequence can be found at, for example, GenBank Accession No. GI: 15011922 (NM_005117.2; SEQ ID NO:1). The sequence of a human FGF19 polypeptide sequence can be found at, for example, GenBank Accession No. GI:4826726 (NP_005108.1; SEQ ID NO: 2). The sequence of an exemplary human FGF19 nucleic acid sequence is Genebank sequence AB018122, AF110400, AY358302, BC017664, and/or BT006729 or an exemplary human FGF19 amino acid sequence is Genebank sequence NP005108.1. In a preferred embodiment, the methods of the invention include inhibiting human FGF19 in order to increase glucose levels in a human subject.

The invention also includes compositions comprising antibodies that bind to FGF19, and/or an FGF19 receptor e.g., klotho and/or FGFR4, or polypeptide or antigen-binding fragments thereof, for use in treating or preventing hypoglycemia, including for example, in a subject having PBH. Thus, in one embodiment, the FGF19 antagonist is an inhibitor of FGF19, which may include, e.g., compositions that inhibit the expression or functional activity of FGF19. Such inhibitors can target FGF19 directly, or can target receptors which bind FGF19 and consequently mediate FGF19 function. Exemplary inhibitors of FGF19 can include, but are not limited to, antagonistic anti-FGF19 antibodies (or antigen binding fragments thereof), soluble forms of an FGF19 receptor, small molecule inhibitors of FGF19, antisense oligonucleotides targeting FGF19, siRNA or shRNA targeting FGF19, and/or inhibitory aptamers that specifically bind FGF19.

In one embodiment, the invention provides methods of treating or preventing hypoglycemia in a subject in need thereof by administering an antagonist of FGF19 which is an antibody, or an antigen binding fragment thereof, which specifically binds to FGF19 and inhibits FGF19 activity or prevents its binding to an FGF19 receptor, such as FGFR4. In one embodiment, the methods of the invention include the use of an anti-FGF19 antibody comprising (a) a light chain comprising: (i) hypervariable region (HVR)-L1 comprising sequence A1-A11, wherein A1-A11 is KASQDINSFLA (SEQ ID NO:29); (ii) HVR-L2 comprising sequence B1-B7, wherein B1-B7 is RANRLVD (SEQ ID NO:30), RANRLVS (SEQ ID NO:31), or RANRLVE (SEQ ID NO:32); and (iii) HVR-L3 comprising sequence C1-C9, wherein C1-C9 is LQYDEFPLT (SEQ ID NO:33); and (b) a heavy chain comprising: (i) HVR-H1 comprising sequence D1-D10, wherein D1-D10 is GFSLTTYGVH (SEQ ID NO:34); (ii) HVR-H2 comprising sequence E1-E17, wherein E1-E17 is GVIWPGGGTDYNAAFIS (SEQ ID NO:35); and (iii) HVR-H3 comprising sequence F1-F13, wherein F1-F13 is VRKEYANLYAMDY (SEQ ID NO:36) (as described in US 2013/0183294 (Genentech), the entire contents of which are incorporated by reference herein). In another embodiment, the anti-FGF19 antibody is humanized In a further embodiment, the anti-FGF19 antibody is humanized anti-FGF19 antibody 1A6.v1 (see U.S. Pat. No. 8,236,307 (Genentech), which is incorporated herein by reference in its entirety). In another embodiment, an anti-FGF19 antibody for use in any of the methods described herein is an anti-FGF19 antibody described in U.S. Pat. Nos. 8,236,307; 7,678,373; U.S. Patent Appln. Publication No. 2005/0026243 A1; U.S. Patent Appln. Publication No. US 2013/0183294 and U.S. Pat. No. 8,409,579. All of the foregoing patent applications are incorporated herein by reference in their entirety.

In another embodiment, the antagonist of FGF19 is a small molecule inhibitor specific for FGF19. In another embodiment, the antagonist of FGF19 is an antisense oligonucleotide specific for FGF19 or an inhibitory aptamer that specifically binds FGF19.

FGF19 stimulates glucose uptake in adipocytes and its activity requires the presence of FGF19 receptors, including klotho (KLB) and fibroblast growth factor receptor 4 (FGFR4).

The term “FGFR4”, as used herein, refers to a native FGFR4 from any vertebrate source, including mammals such as primates (e.g., humans), unless otherwise indicated. In one embodiment, human FGFR4 is inhibited in order to inhibit FGF19 activity in a human subject such that hypoglycemia is treated. The term FGFR4 encompasses full-length, unprocessed FGFR4, as well as any form of FGFR4 that results from processing in a cell. The term also encompasses naturally occurring variants of FGFR4, such as splice variants or allelic variants. The sequence of an exemplary human FGFR4 nucleic acid sequence is provided as SEQ ID NO:3, and the sequence of an exemplary human FGFR4 amino acid sequence is provided herein as SEQ ID NO:4. The sequence of an exemplary human FGFR4 nucleic acid sequence is Genebank sequence AB209631, AF202063, AF359241, AF359246, AF487555, AK301169, BC011847, EF571596, EU826602, EU826603, L03840, M59373, X57205, and/or Y13901 or an exemplary human FGFR4 amino acid sequence is Genebank sequence NP998812.1.

The terms “klotho,” “β-klotho,” and “KLB,” as used herein, refer to a native β-klotho from any vertebrate source, including mammals such as primates (e.g., humans), unless otherwise indicated. The term encompasses full-length, unprocessed β-klotho, as well as any form ofβ-klotho that results from processing in a cell. The term also encompasses naturally occurring variants of β-klotho, such as splice variants or allelic variants. The sequence of an exemplary human β-klotho nucleic acid sequence is provided herein as SEQ ID NO:5, and the sequence of an exemplary human β-klotho amino acid sequence is provided herein as SEQ ID NO:6. The sequence of an exemplary human KLB nucleic acid sequence is Genebank sequence AB079373, AK302436, BC033021, BC104871, and/or BC113653 or an exemplary human KLB amino acid sequence is Genebank sequence NP783864.1.

In some embodiments, the FGF19 inhibitors are antibodies that bind to an FGF19 receptor, e.g, Klotho and/or FGFR4, or antigen-binding fragments thereof. In other embodiments, the antibodies that bind to an FGF19 receptor, are antagonistic antibodies or antigen-binding fragments thereof. In another embodiment, the antagonistic anti-FGF19 receptor antibodies or antigen-binding fragments thereof, are chimeric, humanized or fully human antibodies, or antigen-binding fragments thereof. Examples of anti-FGF19 receptor antibodies for use in any of the methods described herein include an anti-FGF19 reeptor antibody described in PCT Publication Nos. WO2014/105849 and WO2012/174476, which are incorporated herein by reference in their entirety.

In another embodiment, the antagonist of FGF19 is a soluble form of an FGF19 receptor, such as FGFR4 or KLB. In exemplary embodiments, the soluble form of an FGF19 receptor contains all or a portion of the extracellular domain that is sufficient to bind FGF19, and lacks the transmembrane domain which serves to anchor the FGF19 receptor to the cell surface. A soluble form of an FGF19 receptor can inhibit the activity of FGF19 by binding and sequestering FGF19.

### IGFBP1

In one embodiment of the invention, an inhibitor of IGFBP1 is used in the methods and compositions of the invention. IGFBP1 is also known as insulin-like growth factor binding protein 1, placental protein 12, Alpha-Pregnancy-Associated Endometrial Globulin, Growth Hormone Independent-Binding Protein, Amniotic Fluid Binding Protein, IBP-1, PP12, IGF-BP25, HIGFBP-1, AFBP, binding protein 28, binding protein 26, and binding protein 25. The sequence of a human IGFBP1 mRNA can be found, for example, at GenBank Accession GI:61744447 (NM_000596.2; SEQ ID NO: 7). The sequence of a human IGFBP1 polypeptide sequence can be found, for example, at GenBank Accession No. GI:4504615 (NP_000587.1; SEQ ID NO: 8). The term “IGFBP1”, as used herein, refers to a native IGFBP1 from any vertebrate source, including mammals such as primates (e.g., humans), unless otherwise indicated. The term encompasses full-length, unprocessed IGFBP1, as well as any form of IGFBP1that results from processing in a cell. The term also encompasses naturally occurring variants of IGFBP1, such as splice variants or allelic variants.

In one embodiment, the invention includes methods and compositions comprising antibodies that bind to IGFBP1 for use in treating or preventing hypoglycemia, including for example, in a subject having PBH. Thus, in one embodiment, the IGFBP1 antagonist is an inhibitor of IGFBP1, which may include, e.g., compositions that inhibit the expression or functional activity of IGFBP1. Such inhibitors can target IGFBP1 directly, or can target molecules that mediate IGFBP1function. Exemplary inhibitors of IGFBP1 include, but are not limited to, antagonistic anti-IGFBP1 antibodies (or antigen binding fragments thereof), small molecule inhibitors of IGFBP1, antisense oligonucleotides targeting IGFBP1, siRNA or shRNA targeting IGFBP1, and/or inhibitory aptamers that specifically bind IGFBP1.

In one embodiment, the invention provides methods of treating or preventing hypoglycemia in a subject in need thereof by administering an antagonist of IGFBP1 which is an antibody, or an antigen binding fragment thereof, which specifically binds to IGFBP1 and inhibits IGFBP1 activity.

### ADIPOQ

In one embodiment of the invention, an inhibitor of ADIPOQ is used in the methods and compositions of the invention. ADIPOQ is also known as Adiponectin, C1Q And Collagen Domain Containing, GBP28, APM1, adipose most abundant gene transcript 1 protein, 30 kDa adipocyte complement-related protein, ACRP30, Gelatin-Binding Protein and ACDC. The sequence of a human ADIPOQ mRNA can be found, for example, at GenBank Accession GI:295317371 (NM_001177800.1; SEQ ID NO: 9). The sequence of a human ADIPOQ polypeptide sequence can be found, for example, at GenBank Accession No. GI:295317372 (NP_001171271.1; SEQ ID NO: 10). The term “ADIPOQ”, as used herein, refers to a native ADIPOQ from any vertebrate source, including mammals such as primates (e.g., humans), unless otherwise indicated. The term encompasses full-length, unprocessed ADIPOQ, as well as any form of ADIPOQ that results from processing in a cell. The term also encompasses naturally occurring variants of ADIPOQ, such as splice variants or allelic variants.

In one embodiment, the invention includes methods comprising antibodies that bind to ADIPOQ for use in treating or preventing hypoglycemia, including for example, in a subject having PBH. Thus, in one embodiment, the ADIPOQ antagonist is an inhibitor of ADIPOQ, which may include, e.g., compositions that inhibit the expression or functional activity of ADIPOQ. Such inhibitors can target ADIPOQ directly, or can target molecules that mediate ADIPOQ function. Exemplary inhibitors of ADIPOQ include, but are not limited to, antagonistic anti-ADIPOQ antibodies (or antigen binding fragments thereof), small molecule inhibitors of ADIPOQ, antisense oligonucleotides targeting ADIPOQ, siRNA or shRNA targeting ADIPOQ, and/or inhibitory aptamers that specifically bind ADIPOQ.

In one embodiment, the invention provides methods of treating or preventing hypoglycemia in a subject in need thereof by administering an antagonist of ADIPOQ which is an antibody, or an antigen binding fragment thereof, which specifically binds to ADIPOQ and inhibits ADIPOQ activity.

### GCG

In one embodiment of the invention, an inhibitor of GCG is used in the methods and compositions of the invention. GCG is also known as Glicentin-Related Polypeptide, glucagon-like peoptide, GLP1, GLP2, or GRPP. The sequence of a human GCG mRNA can be found, for example, at GenBank Accession GI:389565481 (NM_002054.4; SEQ ID NO: 11). The sequence of a human GCG polypeptide sequence can be found, for example, at GenBank Accession No. GI:4503945 (NP_002045.1; SEQ ID NO: 12). The term “GCG”, as used herein, refers to a native GCG from any vertebrate source, including mammals such as primates (e.g., humans), unless otherwise indicated. The term encompasses full-length, unprocessed GCG, as well as any form of GCG that results from processing in a cell. The term also encompasses naturally occurring variants of GCG, such as splice variants or allelic variants.

In one embodiment, the invention includes methods and compositions comprising antibodies that bind to GCG for use in treating or preventing hypoglycemia, including, for example, in a subject having PBH. Thus, in one embodiment, the GCG antagonist is an inhibitor of GCG, which may include, e.g., compositions that inhibit the expression or functional activity of GCG. Such inhibitors can target GCG directly, or can target molecules that mediate GCG function. Exemplary inhibitors of GCG include, but are not limited to, antagonistic anti-GCG antibodies (or antigen binding fragments thereof), small molecule inhibitors of GCG, antisense oligonucleotides targeting GCG, siRNA or shRNA targeting GCG, and/or inhibitory aptamers that specifically bind GCG.

In one embodiment, the invention provides methods of treating or preventing hypoglycemia in a subject in need thereof by administering an antagonist of GCG which is an antibody, or an antigen binding fragment thereof, which specifically binds to GCG and inhibits GCG activity.

### SHBG

In one embodiment of the invention, an inhibitor of SHBG is used in the methods and compositions of the invention. SHBG is also known as Sex Hormone-Binding Globulin, Testis-Specific Androgen-Binding Protein, Testosterone-Estrogen-Binding Globulin, Sex steroidbinding protein, TEBG, SBP. The sequence of a human SHBG mRNA can be found, for example, at GenBank Accession GI:574287536 (NM_001040.4; SEQ ID NO: 13). The sequence of a human SHBG polypeptide sequence can be found, for example, at GenBank Accession No. GI:7382460 (NP_001031.2; SEQ ID NO: 14). The term “SHBG”, as used herein, refers to a native SHBG from any vertebrate source, including mammals such as primates (e.g., humans), unless otherwise indicated. The term encompasses full-length, unprocessed SHBG, as well as any form of SHBG that results from processing in a cell. The term also encompasses naturally occurring variants of SHBG, such as splice variants or allelic variants.

In one embodiment, the invention includes methods and compositions comprising antibodies that bind to SHBG for use in treating or preventing hypoglycemia, including for example, in a subject having PBH. Thus, in one embodiment, the SHBG antagonist is an inhibitor of SHBG, which may include, e.g., compositions that inhibit the expression or functional activity of SHBG. Such inhibitors can target SHBG directly, or can target molecules that mediate SHBG function. Exemplary inhibitors of SHBG include, but are not limited to, antagonistic anti-SHBG antibodies (or antigen binding fragments thereof), small molecule inhibitors of SHBG, antisense oligonucleotides targeting SHBG, siRNA or shRNA targeting SHBG, and/or inhibitory aptamers that specifically bind SHBG.

In one embodiment, the invention provides methods of treating or preventing hypoglycemia in a subject in need thereof by administering an antagonist of SHBG which is an antibody, or an antigen binding fragment thereof, which specifically binds to SHBG and inhibits SHBG activity.

### 2. Glucose Modulating Molecules: Inflammation Regulators

In one embodiment, the glucose modulating molecule is an inflammation regulator. Inhibitors or antagonists of an inflammation regulator may be used to increase glucose level and treat or prevent hypoglycemia in a subject in need thereof. Examples of inflammation regulators include CXCL3, CXCL2, TNFRSF17, and AMICA1.

### CXCL3

In one embodiment of the invention, an inhibitor of CXCL3 is used in the methods and compositions of the invention. CXCL3 is also known as Chemokine (C—X—C Motif) Ligand 3, Macrophage Inflammatory Protein 2-Beta, Growth-Regulated Protein Gamma, MIP2B, SCYB3, GROg. The sequence of a human CXCL3 mRNA can be found, for example, at GenBank Accession GI:54144649 (NM_002090.2; SEQ ID NO: 15). The sequence of a human CXCL3 polypeptide sequence can be found, for example, at GenBank Accession No. GI:54144650 (NP_002081.2; SEQ ID NO: 16). The term “CXCL3”, as used herein, refers to a native CXCL3 from any vertebrate source, including mammals such as primates (e.g., humans), unless otherwise indicated. The term encompasses full-length, unprocessed CXCL3, as well as any form of CXCL3 that results from processing in a cell. The term also encompasses naturally occurring variants of CXCL3, such as splice variants or allelic variants.

In one embodiment, the invention includes methods and compositions comprising antibodies that bind to CXCL3 for use in treating or preventing hypoglycemia, including for example, in a subject having PBH. Thus, in one embodiment, the CXCL3 antagonist is an inhibitor of CXCL3, which may include, e.g., compositions that inhibit the expression or functional activity of CXCL3. Such inhibitors can target CXCL3 directly, or can target molecules that mediate CXCL3 function. Exemplary inhibitors of CXCL3 include, but are not limited to, antagonistic anti-CXCL3 antibodies (or antigen binding fragments thereof), small molecule inhibitors of CXCL3, antisense oligonucleotides targeting CXCL3, siRNA or shRNA targeting CXCL3, and/or inhibitory aptamers that specifically bind CXCL3.

In one embodiment, the invention provides methods of treating or preventing hypoglycemia in a subject in need thereof by administering an antagonist of CXCL3 which is an antibody, or an antigen binding fragment thereof, which specifically binds to CXCL3 and inhibits CXCL3 activity.

### CXCL2

In one embodiment of the invention, an inhibitor of CXCL2 is used in the methods and compositions of the invention. CXCL2 is also known as Chemokine (C—X—C Motif) Ligand 2, Macrophage Inflammatory Protein 2-Alpha, Growth-Regulated Protein Beta, MIP2A, GRO2, SCYB2. The sequence of a human CXCL2 mRNA can be found, for example, at GenBank Accession GI:148298657 (NM_002089.3; SEQ ID NO: 17). The sequence of a human CXCL2 polypeptide sequence can be found, for example, at GenBank Accession No. GI:4504155 (NP_002080.1; SEQ ID NO: 18). The term “CXCL2”, as used herein, refers to a native CXCL2 from any vertebrate source, including mammals such as primates (e.g., humans), unless otherwise indicated. The term encompasses full-length, unprocessed CXCL2, as well as any form of CXCL2 that results from processing in a cell. The term also encompasses naturally occurring variants of CXCL2, such as splice variants or allelic variants.

In one embodiment, the invention includes methods and compositions comprising antibodies that bind to CXCL2 for use in treating or preventing hypoglycemia, including for example, in a subject having PBH. Thus, in one embodiment, the CXCL2 antagonist is an inhibitor of CXCL2, which may include, e.g., compositions that inhibit the expression or functional activity of CXCL2. Such inhibitors can target CXCL2 directly, or can target molecules that mediate CXCL2 function. Exemplary inhibitors of CXCL2 include, but are not limited to, antagonistic anti-CXCL2 antibodies (or antigen binding fragments thereof), small molecule inhibitors of CXCL2, antisense oligonucleotides targeting CXCL2, siRNA or shRNA targeting CXCL2, and/or inhibitory aptamers that specifically bind CXCL2.

In one embodiment, the invention provides methods of treating or preventing hypoglycemia in a subject in need thereof by administering an antagonist of CXCL2 which is an antibody, or an antigen binding fragment thereof, which specifically binds to CXCL2 and inhibits CXCL2 activity.

### TNFRSF17

In one embodiment of the invention, an inhibitor of TNFRSF17 is used in the methods and compositions of the invention. TNFRSF17 is also known as Tumor Necrosis Factor Receptor Superfamily, Member 17, B-Cell Maturation Protein, B Cell Maturation Antigen, CBMA, CBM, CD269. The sequence of a human TNFRSF17 mRNA can be found, for example, at GenBank Accession GI:23238191 (NM_001192.2; SEQ ID NO: 19). The sequence of a human TNFRSF17 polypeptide sequence can be found, for example, at GenBank Accession No. GI:23238192 (NP_001183.2; SEQ ID NO: 20). The term “TNFRSF17”, as used herein, refers to a native TNFRSF17 from any vertebrate source, including mammals such as primates (e.g., humans), unless otherwise indicated. The term encompasses full-length, unprocessed TNFRSF17, as well as any form of TNFRSF17 that results from processing in a cell. The term also encompasses naturally occurring variants of TNFRSF17, such as splice variants or allelic variants.

In one embodiment, the invention includes methods and compositions comprising antibodies that bind to TNFRSF17 for use in treating or preventing hypoglycemia, including for example, in a subject having PBH. Thus, in one embodiment, the TNFRSF17 antagonist is an inhibitor of TNFRSF17, which may include, e.g., compositions that inhibit the expression or functional activity of TNFRSF17. Such inhibitors can target TNFRSF17 directly, or can target molecules that mediate TNFRSF17 function. Exemplary inhibitors of TNFRSF17 include, but are not limited to, antagonistic anti-TNFRSF17 antibodies (or antigen binding fragments thereof), small molecule inhibitors of TNFRSF17, antisense oligonucleotides targeting TNFRSF17, siRNA or shRNA targeting TNFRSF17, and/or inhibitory aptamers that specifically bind TNFRSF17.

In one embodiment, the invention provides methods of treating or preventing hypoglycemia in a subject in need thereof by administering an antagonist of TNFRSF17 which is an antibody, or an antigen binding fragment thereof, which specifically binds to TNFRSF17 and inhibits TNFRSF17 activity.

### AMICA1

In one embodiment of the invention, an inhibitor of AMICA1 is used in the methods and compositions of the invention. AMICA1 is also known as Adhesion Molecule, Interacts With CXADR Antigen 1, Dendritic-Cell Specific Protein CREA7-1, Junctional Adhesion Molecule-Like, CREA7-1, JAML, Gm638. The sequence of a human AMICA1 mRNA can be found, for example, at GenBank Accession GI:148664206 (NM_001098526.1; SEQ ID NO: 21). The sequence of a human AMICA1 polypeptide sequence can be found, for example, at GenBank Accession No. GI:148664207 (NP_001091996.1; SEQ ID NO: 22). The term “AMICA1”, as used herein, refers to a native AMICA1 from any vertebrate source, including mammals such as primates (e.g., humans), unless otherwise indicated. The term encompasses full-length, unprocessed AMICA1, as well as any form of AMICA1 that results from processing in a cell. The term also encompasses naturally occurring variants of AMICA1, such as splice variants or allelic variants.

In one embodiment, the invention includes methods and compositions comprising antibodies that bind to AMICA1 for use in treating or preventing hypoglycemia, including for example, in a subject having PBH. Thus, in one embodiment, the AMICA1 antagonist is an inhibitor of AMICA1, which may include, e.g., compositions that inhibit the expression or functional activity of AMICA1. Such inhibitors can target AMICA1 directly, or can target molecules that mediate AMICA1 function. Exemplary inhibitors of AMICA1 include, but are not limited to, antagonistic anti-AMICA1 antibodies (or antigen binding fragments thereof), small molecule inhibitors of AMICA1, antisense oligonucleotides targeting AMICA1, siRNA or shRNA targeting AMICA1, and/or inhibitory aptamers that specifically bind AMICA1.

In one embodiment, the invention provides methods of treating or preventing hypoglycemia in a subject in need thereof by administering an antagonist of AMICA1 which is an antibody, or an antigen binding fragment thereof, which specifically binds to AMICA1 and inhibits AMICA1 activity.

### 3. Glucose Modulating Molecules: Developmental Regulators

In one embodiment, the glucose modulating molecule is a developmental regulator. Inhibitors or antagonists of a developmental regulator may be used to increase glucose level and treat or prevent hypoglycemia in a subject in need thereof. Examples of developmental regulators include TFF, EFNB3, and LSAMP.

### TFF3

In one embodiment of the invention, an inhibitor of TFF3 is used in the methods and compositions of the invention. TFF3 is also known as Trefoil Factor 3 (Intestinal), Polypeptide P1.B, Trefoil Factor 3, P1B, TF1. The sequence of a human TFF3 mRNA can be found, for example, at GenBank Accession GI:281485607 (NM_003226.3; SEQ ID NO: 23). The sequence of a human TFF3 polypeptide sequence can be found, for example, at GenBank Accession No. GI:281485608 (NP_003217.3; SEQ ID NO: 24). The term “TFF3”, as used herein, refers to a native TFF3 from any vertebrate source, including mammals such as primates (e.g., humans), unless otherwise indicated. The term encompasses full-length, unprocessed TFF3, as well as any form of TFF3 that results from processing in a cell. The term also encompasses naturally occurring variants of TFF3, such as splice variants or allelic variants.

In one embodiment, the invention includes methods and compositions comprising antibodies that bind to TFF3 for use in treating or preventing hypoglycemia, including for example, in a subject having PBH. Thus, in one embodiment, the TFF3 antagonist is an inhibitor of TFF3, which may include, e.g., compositions that inhibit the expression or functional activity of TFF3. Such inhibitors can target TFF3 directly, or can target molecules that mediate TFF3 function. Exemplary inhibitors of TFF3 include, but are not limited to, antagonistic anti-TFF3 antibodies (or antigen binding fragments thereof), small molecule inhibitors of TFF3, antisense oligonucleotides targeting TFF3, siRNA or shRNA targeting TFF3, and/or inhibitory aptamers that specifically bind TFF3.

In one embodiment, the invention provides methods of treating or preventing hypoglycemia in a subject in need thereof by administering an antagonist of TFF3 which is an antibody, or an antigen binding fragment thereof, which specifically binds to TFF3 and inhibits TFF3 activity.

### EFNB3

In one embodiment of the invention, an inhibitor of EFNB3 is used in the methods and compositions of the invention. EFNB3 is also known as EPH-Related Receptor Transmembrane Ligand ELK-L3, Eph-Related Receptor Tyrosine Kinase Ligand 8, EPLG8, LERK8, EFL6. The sequence of a human EFNB3 mRNA can be found, for example, at GenBank Accession GI:38201712 (NM_001406.3; SEQ ID NO: 25). The sequence of a human EFNB3 polypeptide sequence can be found, for example, at GenBank Accession No. GI:4503489 (NP_001397.1; SEQ ID NO: 26). The term “EFNB3”, as used herein, refers to a native EFNB3 from any vertebrate source, including mammals such as primates (e.g., humans), unless otherwise indicated. The term encompasses full-length, unprocessed EFNB3, as well as any form of EFNB3 that results from processing in a cell. The term also encompasses naturally occurring variants of EFNB3, such as splice variants or allelic variants.

In one embodiment, the invention includes methods and compositions comprising antibodies that bind to EFNB3 for use in treating or preventing hypoglycemia, including for example, in a subject having PBH. Thus, in one embodiment, the EFNB3 antagonist is an inhibitor of EFNB3, which may include, e.g., compositions that inhibit the expression or functional activity of EFNB3. Such inhibitors can target EFNB3 directly, or can target molecules that mediate EFNB3 function. Exemplary inhibitors of EFNB3 include, but are not limited to, antagonistic anti-EFNB3 antibodies (or antigen binding fragments thereof), small molecule inhibitors of EFNB3, antisense oligonucleotides targeting EFNB3, siRNA or shRNA targeting EFNB3, and/or inhibitory aptamers that specifically bind EFNB3.

In one embodiment, the invention provides methods of treating or preventing hypoglycemia in a subject in need thereof by administering an antagonist of EFNB3 which is an antibody, or an antigen binding fragment thereof, which specifically binds to EFNB3 and inhibits EFNB3 activity.

### LSAMP

In one embodiment of the invention, an inhibitor of LSAMP is used in the methods and compositions of the invention. LSAMP is also known as Limbic System-Associated Membrane Protein, IgLON family member, IGLON3, LAMP. The sequence of a human LSAMP mRNA can be found, for example, at GenBank Accession GI:257467557 (NM_002338.3; SEQ ID NO: 27). The sequence of a human LSAMP polypeptide sequence can be found, for example, at GenBank Accession No. GI:45594240 (NP_002329.2; SEQ ID NO: 28). The term “LSAMP”, as used herein, refers to a native LSAMP from any vertebrate source, including mammals such as primates (e.g., humans), unless otherwise indicated. The term encompasses full-length, unprocessed LSAMP, as well as any form of LSAMP that results from processing in a cell. The term also encompasses naturally occurring variants of LSAMP, such as splice variants or allelic variants.

In one embodiment, the invention includes methods and compositions comprising antibodies that bind to LSAMP for use in treating or preventing hypoglycemia, including for example, in a subject having PBH. Thus, in one embodiment, the LSAMP antagonist is an inhibitor of LSAMP, which may include, e.g., compositions that inhibit the expression or functional activity of LSAMP. Such inhibitors can target LSAMP directly, or can target molecules that mediate LSAMP function. Exemplary inhibitors of LSAMP include, but are not limited to, antagonistic anti-LSAMP antibodies (or antigen binding fragments thereof), small molecule inhibitors of LSAMP, antisense oligonucleotides targeting LSAMP, siRNA or shRNA targeting LSAMP, and/or inhibitory aptamers that specifically bind LSAMP.

In one embodiment, the invention provides methods of treating or preventing hypoglycemia in a subject in need thereof by administering an antagonist of LSAMP which is an antibody, or an antigen binding fragment thereof, which specifically binds to LSAMP and inhibits LSAMP activity.

In one embodiment, the antagonist of the glucose modulating molecule is an inhibitor of the glucose modulating molecule, which may include, e.g., compositions that inhibit the expression or functional activity of the glucose modulating molecule. Such inhibitors can target the glucose modulating molecule directly, or can target receptors which bind the glucose modulating molecule and consequently mediate the glucose modulating molecule function. Exemplary inhibitors of the glucose modulating molecule can include, but are not limited to, antagonistic antibodies (or antigen binding fragments thereof) specific for the glucose modulating molecule, soluble forms of receptors specific for the glucose modulating molecule, small molecule inhibitors specific for the glucose modulating molecule, antagonistic polynucleotide, e.g., antisense oligonucleotides, siRNA or shRNA specific for the glucose modulating molecule, and/or inhibitory aptamers that specifically bind the glucose modulating molecule.

### 4. Antagonists of Glucose Modulating Molecules

In one embodiment, the invention includes methods of administering antagonists that will reduce glucose modulating molecules whose activity is associated with hypoglycemia. Thus, by inhibiting or reducing activity of these glucose modulating molecules, glucose levels in a subject, e.g., a human subject, will increase, thereby treating or reducing the symptoms associated with hypoglycemia.

### Antagonist Antibodies

The invention contemplates methods and compositions comprising inhibiting antibodies that bind to a glucose modulating molecule, e.g., FGF19, IGFBP1, ADIPOQ, GCG, SHBG, CXCL3, CXCL2, TNFRSF17, AMICA1, TFF3, EFNB3 and LSAMP. In circumstances where the glucose modulating molecule is a ligand, inhibition of the respective receptor is also contemplated as a method for achieving increased glucose levels in a subject having hypoglycemia.

In one embodiment, the antibody, or antigen-binding fragment thereof, is an antagonistic antibody or antigen-binding fragment thereof, specific for the glucose modulating molecule and/or the receptor for the glucose modulating molecule. In one embodiment, the antagonistic antibody or antigen-binding fragment thereof inhibits the activity of the glucose modulating molecule. Examples of glucose modulating molecules that may be targeted by antagonist antibodies are described above, and include FGF19, IGFBP1, ADIPOQ, GCG, SHBG, CXCL3, CXCL2, TNFRSF17, AMICA1, TFF3, EFNB3 and LSAMP.

Antagonistic antibodies or antigen-binding fragments thereof, useful in the invention may be chimeric, humanized or fully human antibodies, or antigen-binding fragments thereof.

In one embodiment, the antagonist antibody used to inhibit activity of a glucose modulating agent described herein, including an anti-FGF19, anti-klotho, or anti-FGFR4 antibody, or antigen binding fragment thereof, increases the blood glucose level or reduce the symptoms of hypoglycemia.

Antibodies specific for the glucose modulating molecules and/or the receptor for the glucose modulating molecules may be identified, screened for (e.g., using phage display), or characterized for their physical/chemical properties and/or biological activities by various assays known in the art (see, for example, Antibodies: A Laboratory Manual, Second edition, Greenfield, ed., 2014). Assays, for example, described in the Examples may be used to identify antibodies having advantageous properties, such as the ability to increase blood glucose level. In one aspect, an antibody for the glucose modulating molecule, e.g., an anti-FGF19 antibody, is tested for its antigen binding activity, e.g., by known methods such as ELISA, Western blot, etc.

Following identification of the antigen of the antibody e.g., ability to bind the glucose modulating molecule, the activity of the antibody may be tested. In one aspect, assays are provided for identifying antibodies specific for the glucose modulating moelcules, thereof having antagonist activity. For example, biological activity may include the ability to activate signal transduction of particular pathways which can be measured, e.g., by determining levels of FGF19-induced downregulation of cyp7α1 was assessed using hepatocellular carcinoma HEP3B cells (Schlessinger, Science 306:1506-1507 (2004))

Following screening and sequencing, antibodies may be produced using recombinant methods and compositions, e.g., as described in U.S. Pat. No. 4,816,567, incorporated by reference herein. An isolated nucleic acid encoding the antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell).

For recombinant production of an antibody specific for the glucose modulating moelcules, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous haculoviral strains have been identified which may be used in conjunction with insect cells, particularly for transfection of Spodoptera frugiperda cells.

Plant cell cultures can also be utilized as hosts, See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Binding Polypeptides

In one embodiment, the antagonist specific for the glucose modulating molecule, e.g., FGF19, IGFBP1, ADIPOQ, GCG, SHBG, CXCL3, CXCL2, TNFRSF17, AMICA1, TFF3, EFNB3 and LSAMP for use in any of the methods described herein are binding polypeptides. In some embodiments, the binding polypeptide for the glucose modulating molecule inhibits the expression and/or activity the glucose modulating molecule, and/or the receptor for the glucose modulating molecule. In some embodiments, the binding polypeptides bind to FGF19 or FGF19 receptor, e.g., Klotho and/or FGFR4. In some embodiments, the FGF19, klotho, and/or FGFR4 binding polypeptide is an FGF19, klotho, and/or FGFR4 binding polypeptide antagonist.

Binding polypeptides may be chemically synthesized using known polypeptide synthesis methodology or may be prepared and purified using recombinant technology. Binding polypeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such binding polypeptides that are capable of binding, preferably specifically, to a target, e.g., a glucose modulating molecule, e.g. FGF19, and/or a receptor specific for a glucose modulating molecule, e.g., klotho and/or FGFR4 polypeptide, as described herein. Binding polypeptides may be identified without undue experimentation using well known techniques. In this regard, it is noted that techniques for screening polypeptide libraries for binding polypeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Nat'l Acad. Sci. USA, 81:3998-4002 (1984); Geysen et al., Proc. Nat'l Acad. Sci. USA, 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668). In one embodiment, the binding polypeptide is a soluble receptor fragment that binds and sequesters FGF19.

### Inhibitory Nucleic Acids

In one embodiment, the antagonists for the glucose modulating molecules for use in any of the methods described herein are inhibitory nucleic acids. A nucleic acid inhibitor can encode a small interference RNA (e.g., an RNAi agent) that targets one or more of the above-mentioned genes, e.g., FGF19, IGFBP1, ADIPOQ, GCG, SHBG, CXCL3, CXCL2, TNFRSF17, AMICA1, TFF3, EFNB3 and LSAMP, and/or a receptor specific for the glucose modulating molecule, and inhibits its expression or activity. The term “RNAi agent” refers to an RNA, or analog thereof, having sufficient sequence complementarity to a target RNA to direct RNA interference. Examples also include a DNA that can be used to make the RNA.

In one embodiment, the nucleic acid inhibitor can encode a small interference RNA (e.g., an RNAi agent) that targets one or more of the above-mentioned genes, e.g., FGF19, klotho, or FGFR4, and inhibits its expression or activity.

RNA Interference: RNA interference (RNAi) refers to a sequence-specific or selective process by which a target molecule (e.g., a target gene, protein or RNA) is down-regulated. Generally, an interfering RNA (“RNAi”) is a double stranded short-interfering RNA (siRNA), short hairpin RNA (shRNA), or single-stranded micro-RNA (miRNA) that results in catalytic degradation of specific mRNAs, and also can be used to lower or inhibit gene expression. RNA interference (RNAi) is a process whereby double-stranded RNA (dsRNA) induces the sequence-specific regulation of gene expression in animal and plant cells and in bacteria (Aravin and Tuschl, FEBS Lett. 26:5830-5840 (2005); Herbert et al., Curr. Opin. Biotech. 19:500-505 (2008); Hutvagner and Zamore, Curr. Opin. Genet. Dev., 12: 225-232 (2002); Sharp, Genes Dev., 15:485-490 (2001); Valencia-Sanchez et al. Genes Dev. 20:515-524 (2006)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), by microRNA (miRNA), functional small-hairpin RNA (shRNA), or other dsRNAs which are expressed in vivo using DNA templates with RNA polymerase II or III promoters (Zeng et al., Mol. Cell 9:1327-1333 (2002); Paddison et al., Genes Dev. 16:948-958 (2002); Denti, et al., Mol. Ther. 10:191-199 (2004); Lee et al., Nature Biotechnol. 20:500-505 (2002); Paul et al., Nature Biotechnol. 20:505-508 (2002); Rossi, Human Gene Ther. 19:313-317 (2008); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Scherer et al., Nucleic Acids Res. 35:2620-2628 (2007); Sui et al., Proc. Natl. Acad. Sci. USA 99(6):5515-5520 (2002).)

siRNA Molecules: The term “short interfering RNA” or “siRNA” (also known as “small interfering RNAs”) refers to an RNA agent, preferably a double-stranded agent, of about 10-50 nucleotides in length, preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery.

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can be transcribed in vitro or in vivo, e.g., shRNA, from a DNA template. The dsRNA molecules can be designed using any method known in the art. Negative control siRNAs should not have significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. The oligonucleotide modifications include, but are not limited to, 2′-O-methyl, 2′-fluoro, 2′-O-methyoxyethyl and phosphorothioate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004)).

In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles). The inhibitory nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

siRNA Delivery: Direct delivery of siRNA in saline or other excipients can silence target genes in tissues, such as the eye, lung, and central nervous system (Bitko et al., Nat. Med. 11:50-55 (2005); Shen et al., Gene Ther. 13:225-234 (2006); Thakker et al., Proc. Natl. Acad. Sci. U.S.A. (2004)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)).

Liposomes and nanoparticles can also be used to deliver siRNA into animals. Delivery methods using liposomes, e.g. stable nucleic acid-lipid particles (SNALPs), dioleoyl phosphatidylcholine (DOPC)-based delivery system, as well as lipoplexes, e.g. Lipofectamine 2000, TransIT-TKO, have been shown to effectively repress target mRNA (de Fougerolles, Human Gene Ther. 19:125-132 (2008); Landen et al., Cancer Res. 65:6910-6918 (2005); Luo et al., Mol. Pain 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake. (Howard et al., Mol. Ther. 14:476-484 (2006); Hu-Lieskovan et al., Cancer Res. 65:8984-8992 (2005); Kumar, et al., Nature 448:39-43; McNamara et al., Nat. Biotechnol. 24:1005-1015 (2007); Rozema et al., Proc. Natl. Acad. Sci. U.S.A. 104:12982-12987 (2007); Song et al., Nat. Biotechnol. 23:709-717 (2005); Soutschek (2004), supra; Wolfrum et al., Nat. Biotechnol. 25:1149-1157 (2007)).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)).

Stable siRNA Expression: Synthetic siRNAs can be delivered into cells, e.g., by direct delivery, cationic liposome transfection, and electroporation. However, these exogenous siRNA typically only show short term persistence of the silencing effect (4-5 days). Several strategies for expressing siRNA duplexes within cells from recombinant DNA constructs allow longer-term target gene suppression in cells, including mammalian Pol II and III promoter systems (e.g., H1, U1, or U6/snRNA promoter systems (Denti et al. (2004), supra; Tuschl (2002), supra); capable of expressing functional double-stranded siRNAs (Bagella et al., J. Cell. Physiol. 177:206-213 (1998); Lee et al. (2002), supra; Miyagishi et al. (2002), supra; Paul et al. (2002), supra; Scherer et al. (2007), supra; Yu et al. (2002), supra; Sui et al. (2002), supra).

Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

In another embodiment, siRNAs can be expressed in a miRNA backbone which can be transcribed by either RNA Pol II or III. MicroRNAs are endogenous noncoding RNAs of approximately 22 nucleotides in animals and plants that can post-transcriptionally regulate gene expression (Bartel, Cell 116:281-297 (2004); Valencia-Sanchez et al., Genes & Dev. 20:515-524 (2006)). One common feature of miRNAs is that they are excised from an approximately 70 nucleotide precursor RNA stem loop by Dicer, an RNase III enzyme, or a homolog thereof. By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression (McManus (2002), supra; Zeng (2002), supra).

Uses of Engineered RNA Precursors to Induce RNAi: Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage, destabilization, and/or translation inhibition destruction. In this fashion, the mRNA to be targeted by the siRNA generated from the engineered RNA precursor will be depleted from the cell or organism, leading to a decrease in the concentration of the protein encoded by that mRNA in the cell or organism.

Antisense: An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein relating to the identified glucose modulating molecules, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to a regulatory region, e.g., promoters and/or enhancers) to form triple helical structures that prevent transcription of the target gene in target cells. See generally, Helene, C. Anticancer Drug Des. 6:569-84 (1991); Helene, C. Ann. N.Y. Acad. Sci. 660:27-36 (1992); and Maher, Bioassays. 14:807-15 (1992). The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In one embodiment, the antagonists for the glucose modulating molecules for use in any of the methods described herein are test compounds. Test compounds that act as an inhibitor for the glucose modulating molecule, e.g., FGF19, IGFBP1, ADIPOQ, GCG, SHBG, CXCL3, CXCL2, TNFRSF17, AMICA1, TFF3, EFNB3 and LSAMP, and/or a receptor specific for the glucose modulating molecule, can be identified through screening assays. The test compounds can be, e.g., natural products or members of a combinatorial chemistry library.

In some embodiments, the test compounds that act as antagonists for the glucose modulating molecules are small molecules. In some embodiments, the antagonists for the glucose modulating molecules inhibits the expression and/or activity of the glucose modulating molecule and/or the receptor specific for the glucose modulating molecule. In some embodiments, the small molecule binds to the glucose modulating molecule. In some embodiments, the small molecule binds to a receptor for the glucose modulating molecule.

### Small Molecule Inhibitors

In some embodiments, the antagonist for the glucose modulating molecule is a small molecule inhibitor. In some embodiments, the small molecule inhibitor binds to the glucose modulating molecule. In some embodiments, the small molecule inhibitor binds to a receptor for the glucose modulating molecule.

In some embodiments, the FGF19 antagonist is a small molecule inhibitor specific for FGF19. In other embodiments, the FGF19 antagonist is a small molecule inhibitor specific for klotho. In another embodiment, the FGF19 antagonist is a small molecule inhibitor specific for FGFR4. In some embodiments, the small molecule inhibitors specific for FGFR4 for use in any of the methods described herein are small molecule inhibitors described in PCT Publication No. WO2015/030021, which is incorporated by reference in its entirety.

In some embodiments, the IGFBP1 antagonist is a small molecule inhibitor specific for IGFBP1. In other embodiments, the IGFBP1 antagonist is a small molecule inhibitor specific for a receptor of IGFBP1. In some embodiments, the ADIPOQ antagonist is a small molecule inhibitor specific for ADIPOQ. In other embodiments, the ADIPOQ antagonist is a small molecule inhibitor specific for a receptor of ADIPOQ. In some embodiments, the GCG antagonist is a small molecule inhibitor specific for GCG. In other embodiments, the GCG antagonist is a small molecule inhibitor specific for a receptor of GCG. In some embodiments, the SHBG antagonist is a small molecule inhibitor specific for SHBG. In other embodiments, the SHBG antagonist is a small molecule inhibitor specific for a receptor of SHBG. In some embodiments, the CXCL3 antagonist is a small molecule inhibitor specific for CXCL3. In other embodiments, the CXCL3 antagonist is a small molecule inhibitor specific for a receptor of CXCL3. In some embodiments, the CXCL2 antagonist is a small molecule inhibitor specific for CXCL2. In other embodiments, the CXCL2 antagonist is a small molecule inhibitor specific for a receptor of CXCL2. In some embodiments, the TNFRSF17 antagonist is a small molecule inhibitor specific for TNFRSF17. In other embodiments, the TNFRSF17 antagonist is a small molecule inhibitor specific for a receptor of TNFRSF17. In some embodiments, the AMICA1 antagonist is a small molecule inhibitor specific for AMICA1. In other embodiments, the AMICA1 antagonist is a small molecule inhibitor specific for a receptor of AMICA1. In some embodiments, the TFF3 antagonist is a small molecule inhibitor specific for TFF3. In other embodiments, the TFF3 antagonist is a small molecule inhibitor specific for a receptor of TFF3. In some embodiments, the EFNB3 antagonist is a small molecule inhibitor specific for EFNB3. In other embodiments, the EFNB3 antagonist is a small molecule inhibitor specific for a receptor of EFNB3. In some embodiments, the LSAMP antagonist is a small molecule inhibitor specific for LSAMP. In other embodiments, the LSAMP antagonist is a small molecule inhibitor specific for a receptor of LSAMP.

In some embodiments, the test compounds are initially members of a library, e.g., an inorganic or organic chemical library, peptide library, oligonucleotide library, or mixed-molecule library. In some embodiments, the methods include screening small molecules, e.g., natural products or members of a combinatorial chemistry library. These methods can also be used, for example, to screen a library of proteins or fragments thereof, e.g., proteins that are expressed in liver or pancreatic cells.

A given library can comprise a set of structurally related or unrelated test compounds. Preferably, a set of diverse molecules should be used to cover a variety of functions such as charge, aromaticity, hydrogen bonding, flexibility, size, length of side chain, hydrophobicity, and rigidity. Combinatorial techniques suitable for creating libraries are known in the art, e.g., methods for synthesizing libraries of small molecules, e.g., as exemplified by Obrecht and Villalgordo, Solid-Supported Combinatorial and Parallel Synthesis of Small-Molecular-Weight Compound Libraries, Pergamon-Elsevier Science Limited (1998). Such methods include the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of libraries, including small molecule libraries, are commercially available.

In some embodiments, the test compounds are peptide or peptidomimetic molecules, e.g., peptide analogs including peptides comprising non-naturally occurring amino acids or having non-peptide linkages; peptidomimetics (e.g., peptoid oligomers, e.g., peptoid amide or ester analogues, .theta.-peptides, D-peptides, L-peptides, oligourea or oligocarbamate); small peptides (e.g., pentapeptides, hexapeptides, heptapeptides, octapeptides, nonapeptides, decapeptides, or larger, e.g., 20-mers or more); cyclic peptides; other non-natural or unnatural peptide-like structures; and inorganic molecules (e.g., heterocyclic ring molecules). In some embodiments, the test compounds are nucleic acids, e.g., DNA or RNA oligonucleotides.

In some embodiments, test compounds and libraries thereof can be obtained by systematically altering the structure of a first test compound. Taking a small molecule as an example, e.g., a first small molecule is selected that is, e.g., structurally similar to a known phosphorylation or protein recognition site. For example, in one embodiment, a general library of small molecules is screened, e.g., using the methods described herein, to select a first test small molecule. Using methods known in the art, the structure of that small molecule is identified if necessary and correlated to a resulting biological activity, e.g., by a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds.

In some embodiments, test compounds identified as “hits” (e.g., test compounds that demonstrate activity in a method described herein) in a first screen are selected and optimized by being systematically altered, e.g., using rational design, to optimize binding affinity, avidity, specificity, or other parameter. Such potentially optimized structures can also be screened using the methods described herein. Thus, in one embodiment, the invention includes screening a first library of test compounds using a method described herein, identifying one or more hits in that library, subjecting those hits to systematic structural alteration to create one or more second generation compounds structurally related to the hit, and screening the second generation compound. Additional rounds of optimization can be used to identify a test compound with a desirable therapeutic profile.

Test compounds identified as hits can be considered candidate therapeutic compounds, useful in treating disorders described herein. Thus, the invention also includes compounds identified as “hits” by a method described herein, and methods for their administration and use in the treatment, prevention, or delay of development or progression of a disease described herein.

### Mimetics

Variants of the glucose modulating molecule, e.g., FGF19, IGFBP1, ADIPOQ, GCG, SHBG, CXCL3, CXCL2, TNFRSF17, AMICA1, TFF3, EFNB3 and LSAMP, and/or a receptor specific for the glucose modulating molecule, by screening combinatorial libraries of mutants. In some embodiment, variants of FGF19, klotho, or FGFR4 that function as FGF19 inhibitors can be identified by screening combinatorial libraries of mutants, e.g., truncation mutants, of FGF19, klotho, or FGFR4.

In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the marker proteins from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

Thus, in a further embodiment, the methods of the invention also may be practiced using a mimetic of an antagonist of the glucose modulating molecules.

### II.B. Glucose Modulating Molecules Whose Expression Levels are Downregulated in Subjects Having Hypoglycemia

One aspect of the present invention features a method of increasing the blood glucose level of a subject in need thereof, comprising administering an agonist of one or more glucose modulating molecule(s) to the subject, wherein the glucose modulating molecule is HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPCS, ARSB or SORCS2, or a combination thereof, such that the blood glucose level of the subject is increased.

In another embodiment, the invention provides methods of treating or reducing the symptoms of hypoglycemia in a subject in need thereof, comprising administering an agonist of one or more glucose modulating molecule(s) to the subject, wherein the glucose modulating molecule is HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPCS, ARSB or SORCS2, or a combination thereof, such that hypoglycemia is treated or reduced.

### 1. Hormone Signaling and Metabolic Regulators

In one embodiment, the glucose modulating molecule is a hormone signaling or metabolic regulator. An activator or agonist of a hormone signaling or metabolic regulator may be used to increase glucose level and treat or prevent hypoglycemia in a subject in need thereof. Examples of hormone signaling or metabolic regulators include HGFAC, BMPR2, GDF11, IGFBP7 and IGFBP6.

### HGFAC

In one embodiment of the invention, an activator of HGFAC is used in the methods and compositions of the invention. HGFAC is also known as HGF activator, HGFA, Hepatocyte growth factor activator, and EC 3.4.21. The sequence of a human HGFAC mRNA can be found, for example, at GenBank Accession GI: 661903022 (NM_001297439.1; SEQ ID NO: 37). The sequence of a human HGFAC polypeptide sequence can be found, for example, at GenBank Accession No. GI: 661903023 (NP_001284368.1; SEQ ID NO: 38). The term “HGFAC”, as used herein, refers to a native HGFAC from any vertebrate source, including mammals such as primates (e.g., humans), unless otherwise indicated. The term encompasses full-length, unprocessed HGFAC, as well as any form of HGFAC that results from processing in a cell. The term also encompasses naturally occurring variants of HGFAC, such as splice variants or allelic variants.

In one embodiment, the invention includes methods and compositions comprising antibodies that bind to HGFAC for use in treating or preventing hypoglycemia, including for example, in a subject having PBH. Thus, in one embodiment, the HGFAC agonist is an activator of HGFAC, which may include, e.g., compositions that activate the expression or functional activity of HGFAC. Such activators can target HGFAC directly, or can target molecules that mediate HGFAC function. Exemplary activators of HGFAC include, but are not limited to, agonistic anti-HGFAC antibodies (or antigen binding fragments thereof), small molecule activators of HGFAC, and/or stimulatory aptamers that specifically bind HGFAC.

In one embodiment, the invention provides methods of treating or preventing hypoglycemia in a subject in need thereof by administering an agonist of HGFAC which is an antibody, or an antigen binding fragment thereof, which specifically binds to HGFAC and activates HGFAC activity. In one embodiment, the methods of modulating glucose described herein include administration of a HGFAC protein or nucleic acid encoding HGFAC.

### BMPR2

In one embodiment of the invention, an activator of BMPR2 is used in the methods and compositions of the invention. BMPR2 is also known as Bone Morphogenetic Protein Receptor, Type II (Serine/Threonine Kinase), PPH1, Bone Morphogenetic Protein Receptor Type II, BMP Type II Receptor, BMP Type-2 Receptor, EC 2.7.11.30, BMPR-II, BMPR-2, POVD1 3, Type II Receptor For Bone Morphogenetic Protein-4, Bone Morphogenetic Protein Receptor Type-2, Type II Activin Receptor-Like Kinase, Primary Pulmonary Hypertension, EC 2.7.11, BMPR3, BRK-3, T-ALK, and BMR2. The sequence of a human BMPR2 mRNA can be found, for example, at GenBank Accession GI: 189339276 (NM_001204.6; SEQ ID NO: 39). The sequence of a human BMPR2 polypeptide sequence can be found, for example, at GenBank Accession No. GI: 15451916 (NP_001195.2 ; SEQ ID NO: 40). The term “BMPR2”, as used herein, refers to a native BMPR2 from any vertebrate source, including mammals such as primates (e.g., humans), unless otherwise indicated. The term encompasses full-length, unprocessed BMPR2, as well as any form of BMPR2 that results from processing in a cell. The term also encompasses naturally occurring variants of BMPR2, such as splice variants or allelic variants.

In one embodiment, the invention includes methods and compositions comprising antibodies that bind to BMPR2 for use in treating or preventing hypoglycemia, including for example, in a subject having PBH. Thus, in one embodiment, the BMPR2 agonist is an activator of BMPR2, which may include, e.g., compositions that activate the expression or functional activity of BMPR2. Such activators can target BMPR2 directly, or can target molecules that mediate BMPR2 function. Exemplary activators of BMPR2 include, but are not limited to, agonistic anti-BMPR2 antibodies (or antigen binding fragments thereof), small molecule activators of BMPR2, and/or stimulatory aptamers that specifically bind BMPR2.

In one embodiment, the invention provides methods of treating or preventing hypoglycemia in a subject in need thereof by administering an agonist of BMPR2 which is an antibody, or an antigen binding fragment thereof, which specifically binds to BMPR2 and activates BMPR2 activity. In one embodiment, the methods of modulating glucose described herein include administration of a BMPR2 protein or nucleic acid encoding BMPR2.

### GDF11

In one embodiment of the invention, an activator of GDF11 is used in the methods and compositions of the invention. GDF11 is also known as Growth Differentiation Factor 11, BMP11, Bone Morphogenetic Protein 11, BMP-11, GDF-11, and Growth/Differentiation Factor 11. The sequence of a human GDF11 mRNA can be found, for example, at GenBank Accession GI: 223941867 (NM_005811.3; SEQ ID NO: 41). The sequence of a human GDF11 polypeptide sequence can be found, for example, at GenBank Accession No. GI: 5031613 (NP_005802.1; SEQ ID NO: 42). The term “GDF11”, as used herein, refers to a native GDF11 from any vertebrate source, including mammals such as primates (e.g., humans), unless otherwise indicated. The term encompasses full-length, unprocessed GDF11, as well as any form of GDF11 that results from processing in a cell. The term also encompasses naturally occurring variants of GDF11, such as splice variants or allelic variants.

In one embodiment, the invention includes methods and compositions comprising antibodies that bind to GDF11 for use in treating or preventing hypoglycemia, including for example, in a subject having PBH. Thus, in one embodiment, the GDF11 agonist is an activator of GDF11, which may include, e.g., compositions that activate the expression or functional activity of GDF11. Such activators can target GDF11 directly, or can target molecules that mediate GDF11 function. Exemplary activators of GDF11 include, but are not limited to, agonistic anti-GDF11 antibodies (or antigen binding fragments thereof), small molecule activators of GDF11, and/or stimulatory aptamers that specifically bind GDF11.

In one embodiment, the invention provides methods of treating or preventing hypoglycemia in a subject in need thereof by administering an agonist of GDF11 which is an antibody, or an antigen binding fragment thereof, which specifically binds to GDF11 and activates GDF11 activity. In one embodiment, the methods of modulating glucose described herein include administration of a GDF11 protein or nucleic acid encoding GDF11.

### IGFBP7

In one embodiment of the invention, an activator of IGFBP7 is used in the methods and compositions of the invention. IGFBP7 is also known as Insulin-Like Growth Factor Binding Protein, MAC25, Prostacyclin-Stimulating Factor, Tumor-Derived Adhesion Factor, PGI2-Stimulating Factor, IGF-Binding Protein, IGFBP-RP1, RAMSVPS, IGFBP-7, IBP-7, TAF, PSF, Insulin-Like Growth Factor-Binding Protein, MAC25 Protein, Angiomodulin, IGFBP-7v, IGFBPRP1, FSTL2, and AGM. The sequence of a human IGFBP7 mRNA can be found, for example, at GenBank Accession GI: 359465607 (NM_001253835.1; SEQ ID NO: 43). The sequence of a human IGFBP7 polypeptide sequence can be found, for example, at GenBank Accession No. GI: 359465608 (NP_001240764.1; SEQ ID NO: 44). The term “IGFBP7”, as used herein, refers to a native IGFBP7 from any vertebrate source, including mammals such as primates (e.g., humans), unless otherwise indicated. The term encompasses full-length, unprocessed IGFBP7, as well as any form of IGFBP7 that results from processing in a cell. The term also encompasses naturally occurring variants of IGFBP7, such as splice variants or allelic variants.

In one embodiment, the invention includes methods and compositions comprising antibodies that bind to IGFBP7 for use in treating or preventing hypoglycemia, including for example, in a subject having PBH. Thus, in one embodiment, the IGFBP7 agonist is an activator of IGFBP7, which may include, e.g., compositions that activate the expression or functional activity of IGFBP7. Such activators can target IGFBP7 directly, or can target molecules that mediate IGFBP7 function. Exemplary activators of IGFBP7 include, but are not limited to, agonistic anti-IGFBP7 antibodies (or antigen binding fragments thereof), small molecule activators of IGFBP7, and/or stimulatory aptamers that specifically bind IGFBP7.

In one embodiment, the invention provides methods of treating or preventing hypoglycemia in a subject in need thereof by administering an agonist of IGFBP7 which is an antibody, or an antigen binding fragment thereof, which specifically binds to IGFBP7 and activates IGFBP7 activity. In one embodiment, the methods of modulating glucose described herein include administration of an IGFBP7 protein or nucleic acid encoding IGFBP7.

### IGFBP6

In one embodiment of the invention, an activator of IGFBP6 is used in the methods and compositions of the invention. IGFBP6 is also known as Insulin-Like Growth Factor Binding Protein 6, IGF-Binding Protein 6, IGFBP-6, IBP-6, IBP6, Insulin-Like Growth Factor-Binding Protein 6, and IGF Binding Protein 6. The sequence of a human IGFBP6 mRNA can be found, for example, at GenBank Accession GI: 49574524 (NM_002178.2; SEQ ID NO: 45). The sequence of a human IGFBP6 polypeptide sequence can be found, for example, at GenBank Accession No. GI: 11321593 (NP_002169.1; SEQ ID NO: 46). The term “IGFBP6”, as used herein, refers to a native IGFBP6 from any vertebrate source, including mammals such as primates (e.g., humans), unless otherwise indicated. The term encompasses full-length, unprocessed IGFBP6, as well as any form of IGFBP6 that results from processing in a cell. The term also encompasses naturally occurring variants of IGFBP6, such as splice variants or allelic variants.

In one embodiment, the invention includes methods and compositions comprising antibodies that bind to IGFBP6 for use in treating or preventing hypoglycemia, including for example, in a subject having PBH. Thus, in one embodiment, the IGFBP6 agonist is an activator of IGFBP6, which may include, e.g., compositions that activate the expression or functional activity of IGFBP6. Such activators can target IGFBP6 directly, or can target molecules that mediate IGFBP6 function. Exemplary activators of IGFBP6 include, but are not limited to, agonistic anti-IGFBP6 antibodies (or antigen binding fragments thereof), small molecule activators of IGFBP6, and/or stimulatory aptamers that specifically bind IGFBP6.

In one embodiment, the invention provides methods of treating or preventing hypoglycemia in a subject in need thereof by administering an agonist of IGFBP6 which is an antibody, or an antigen binding fragment thereof, which specifically binds to IGFBP6 and activates IGFBP6 activity. In one embodiment, the methods of modulating glucose described herein include administration of an IGFBP6 protein or nucleic acid encoding IGFBP6.

### 2. Lipid Metabolism Regulators

In one embodiment, the glucose modulating molecule is a lipid metabolism regulator. An activator or agonist of a lipid metabolism regulator may be used to increase glucose level and treat or prevent hypoglycemia in a subject in need thereof. Examples of lipid metabolism regulators include APOE and PLA2G7.

### APOE

In one embodiment of the invention, an activator of APOE is used in the methods and compositions of the invention. APOE is also known as Apolipoprotein E, LDLCQ5, APO-E, LPG, AD2, Alzheimer Disease 2 (APOE*E4-Associated, Late Onset), and Apolipoprotein E3. The sequence of a human APOE mRNA can be found, for example, at GenBank Accession GI: 705044057 (NM_000041.3; SEQ ID NO: 47). The sequence of a human APOE polypeptide sequence can be found, for example, at GenBank Accession No. GI: 4557325 (NP_000032.1; SEQ ID NO: 48). The term “APOE”, as used herein, refers to a native APOE from any vertebrate source, including mammals such as primates (e.g., humans), unless otherwise indicated. The term encompasses full-length, unprocessed APOE, as well as any form of APOE that results from processing in a cell. The term also encompasses naturally occurring variants of APOE, such as splice variants or allelic variants.

In one embodiment, the invention includes methods and compositions comprising antibodies that bind to APOE for use in treating or preventing hypoglycemia, including for example, in a subject having PBH. Thus, in one embodiment, the APOE agonist is an activator of APOE, which may include, e.g., compositions that activate the expression or functional activity of APOE. Such activators can target APOE directly, or can target molecules that mediate APOE function. Exemplary activators of APOE include, but are not limited to, agonistic anti-APOE antibodies (or antigen binding fragments thereof), small molecule activators of APOE, and/or stimulatory aptamers that specifically bind APOE.

In one embodiment, the invention provides methods of treating or preventing hypoglycemia in a subject in need thereof by administering an agonist of APOE which is an antibody, or an antigen binding fragment thereof, which specifically binds to APOE and activates APOE activity. In one embodiment, the methods of modulating glucose described herein include administration of an APOE protein or nucleic acid encoding APOE.

### PLA2G7

In one embodiment of the invention, an activator of PLA2G7 is used in the methods and compositions of the invention. PLA2G7 is also known as Phospholipase A2, Group VII (Platelet-Activating Factor, Acetylhydrolase, Plasma), PAFAH, 1-Alkyl-2-Acetylglycerophosphocholine Esterase, 2-Acetyl-1-Alkylglycerophosphocholine Esterase, LDL-Associated Phospholipase A2, Group-VIIA Phospholipase A2, PAF 2-Acylhydrolase, PAF Acetylhydrolase, EC 3.1.1.47, LDL-PLA(2), GVIIA-PLA2, PAFAD, Platelet-Activating Factor Acetylhydrolase, Lipoprotein-Associated Phospholipase A2, LDL-PLA2, EC 3.1.1, and LP-PLA2. The sequence of a human PLA2G7 mRNA can be found, for example, at GenBank Accession GI: 270133070 (NM_001168357.1; SEQ ID NO: 49). The sequence of a human PLA2G7 polypeptide sequence can be found, for example, at GenBank Accession No. GI: 270133071 (NP_001161829.1; SEQ ID NO: 50). The term “PLA2G7”, as used herein, refers to a native PLA2G7 from any vertebrate source, including mammals such as primates (e.g., humans), unless otherwise indicated. The term encompasses full-length, unprocessed PLA2G7, as well as any form of PLA2G7 that results from processing in a cell. The term also encompasses naturally occurring variants of PLA2G7, such as splice variants or allelic variants.

In one embodiment, the invention includes methods and compositions comprising antibodies that bind to PLA2G7 for use in treating or preventing hypoglycemia, including for example, in a subject having PBH. Thus, in one embodiment, the PLA2G7 agonist is an activator of PLA2G7, which may include, e.g., compositions that activate the expression or functional activity of PLA2G7. Such activators can target PLA2G7 directly, or can target molecules that mediate PLA2G7 function. Exemplary activators of PLA2G7 include, but are not limited to, agonistic anti-PLA2G7 antibodies (or antigen binding fragments thereof), small molecule activators of PLA2G7, and/or stimulatory aptamers that specifically bind PLA2G7.

In one embodiment, the invention provides methods of treating or preventing hypoglycemia in a subject in need thereof by administering an agonist of PLA2G7 which is an antibody, or an antigen binding fragment thereof, which specifically binds to PLA2G7 and activates PLA2G7 activity. In one embodiment, the methods of modulating glucose described herein include administration of a PLA2G7 protein or nucleic acid encoding PLA2G7.

### 3. Cell Cycle Regulator

In one embodiment, the glucose modulating molecule is a cell cycle regulator. An activator or agonist of a cell cycle regulator may be used to increase glucose level and treat or prevent hypoglycemia in a subject in need thereof. Examples of cell cycle regulators include CDK2, CCNA2 and MAPKAPK3.

### CDK2

In one embodiment of the invention, an activator of CDK2 is used in the methods and compositions of the invention. CDK2 is also known as Cyclin-Dependent Kinase 2, Cell Division Protein Kinase 2, P33 Protein Kinase, EC 2.7.11.22, CDKN2, Cdc2-Related Protein Kinase, P33(CDK2), and EC 2.7.1. The sequence of a human CDK2 mRNA can be found, for example, at GenBank Accession GI: 589811556 (NM_001290230.1; SEQ ID NO: 51). The sequence of a human CDK2 polypeptide sequence can be found, for example, at GenBank Accession No. GI: 589811557 (NP_001277159.1; SEQ ID NO: 52). The term “CDK2”, as used herein, refers to a native CDK2 from any vertebrate source, including mammals such as primates (e.g., humans), unless otherwise indicated. The term encompasses full-length, unprocessed CDK2, as well as any form of CDK2 that results from processing in a cell. The term also encompasses naturally occurring variants of CDK2, such as splice variants or allelic variants.

In one embodiment, the invention includes methods and compositions comprising antibodies that bind to CDK2 for use in treating or preventing hypoglycemia, including for example, in a subject having PBH. Thus, in one embodiment, the CDK2 agonist is an activator of CDK2, which may include, e.g., compositions that activate the expression or functional activity of CDK2. Such activators can target CDK2 directly, or can target molecules that mediate CDK2 function. Exemplary activators of CDK2 include, but are not limited to, agonistic anti-CDK2 antibodies (or antigen binding fragments thereof), small molecule activators of CDK2, and/or stimulatory aptamers that specifically bind CDK2.

In one embodiment, the invention provides methods of treating or preventing hypoglycemia in a subject in need thereof by administering an agonist of CDK2 which is an antibody, or an antigen binding fragment thereof, which specifically binds to CDK2 and activates CDK2 activity. In one embodiment, the methods of modulating glucose described herein include administration of a CDK2 protein or nucleic acid encoding CDK2.

### CCNA2

In one embodiment of the invention, an activator of CCNA2 is used in the methods and compositions of the invention. CCNA2 is also known as Cyclin A2, CCN1, CCNA, Cyclin-A, and Cyclin-A2. The sequence of a human CCNA2 mRNA can be found, for example, at GenBank Accession GI: 166197663 (NM_001237.3; SEQ ID NO: 53). The sequence of a human CCNA2 polypeptide sequence can be found, for example, at GenBank Accession No. GI: 4502613 (NP_001228.1; SEQ ID NO: 54). The term “CCNA2”, as used herein, refers to a native CCNA2 from any vertebrate source, including mammals such as primates (e.g., humans), unless otherwise indicated. The term encompasses full-length, unprocessed CCNA2, as well as any form of CCNA2 that results from processing in a cell. The term also encompasses naturally occurring variants of CCNA2, such as splice variants or allelic variants.

In one embodiment, the invention includes methods and compositions comprising antibodies that bind to CCNA2 for use in treating or preventing hypoglycemia, including for example, in a subject having PBH. Thus, in one embodiment, the CCNA2 agonist is an activator of CCNA2, which may include, e.g., compositions that activate the expression or functional activity of CCNA2. Such activators can target CCNA2 directly, or can target molecules that mediate CCNA2 function. Exemplary activators of CCNA2 include, but are not limited to, agonistic anti-CCNA2 antibodies (or antigen binding fragments thereof), small molecule activators of CCNA2, and/or stimulatory aptamers that specifically bind CCNA2.

In one embodiment, the invention provides methods of treating or preventing hypoglycemia in a subject in need thereof by administering an agonist of CCNA2 which is an antibody, or an antigen binding fragment thereof, which specifically binds to CCNA2 and activates CCNA2 activity. In one embodiment, the methods of modulating glucose described herein include administration of a CCNA2 protein or nucleic acid encoding CCNA2.

### MAPKAPK3

In one embodiment of the invention, an activator of MAPKAPK3 is used in the methods and compositions of the invention. MAPKAPK3 is also known as Mitogen-Activated Protein Kinase-Activated Protein, Kinase 3, 3PK, MAPK-Activated Protein Kinase 3, Chromosome 3p Kinase, MAPKAP Kinase 3, EC 2.7.11.1, MAPKAP-K3, MAPKAPK-3, MAPKAP3, MK-3, MAP Kinase-Activated Protein Kinase 3, and EC 2.7.11. The sequence of a human MAPKAPK3 mRNA can be found, for example, at GenBank Accession GI: 345441755 (NM_001243925.1; SEQ ID NO: 55). The sequence of a human MAPKAPK3 polypeptide sequence can be found, for example, at GenBank Accession No. GI: 345441756 (NP_001230854.1; SEQ ID NO: 56). The term “MAPKAPK3”, as used herein, refers to a native MAPKAPK3 from any vertebrate source, including mammals such as primates (e.g., humans), unless otherwise indicated. The term encompasses full-length, unprocessed MAPKAPK3, as well as any form of MAPKAPK3 that results from processing in a cell. The term also encompasses naturally occurring variants of MAPKAPK3, such as splice variants or allelic variants.

In one embodiment, the invention includes methods and compositions comprising antibodies that bind to MAPKAPK3 for use in treating or preventing hypoglycemia, including for example, in a subject having PBH. Thus, in one embodiment, the MAPKAPK3 agonist is an activator of MAPKAPK3, which may include, e.g., compositions that activate the expression or functional activity of MAPKAPK3. Such activators can target MAPKAPK3 directly, or can target molecules that mediate MAPKAPK3 function. Exemplary activators of MAPKAPK3 include, but are not limited to, agonistic anti-MAPKAPK3 antibodies (or antigen binding fragments thereof), small molecule activators of MAPKAPK3, and/or stimulatory aptamers that specifically bind MAPKAPK3.

In one embodiment, the invention provides methods of treating or preventing hypoglycemia in a subject in need thereof by administering an agonist of MAPKAPK3 which is an antibody, or an antigen binding fragment thereof, which specifically binds to MAPKAPK3 and activates MAPKAPK3 activity. In one embodiment, the methods of modulating glucose described herein include administration of a MAPKAPK3 protein or nucleic acid encoding MAPKAPK3.

### 4. Proteases

In one embodiment, the glucose modulating molecule is a protease. An activator or agonist of a protease may be used to increase glucose level and treat or prevent hypoglycemia in a subject in need thereof. Examples of proteases include KLK3 and PLAT.

### KLK3

In one embodiment of the invention, an activator of KLK3 is used in the methods and compositions of the invention. KLK3 is also known as Kallikrein-Related Peptidase 3, PSA, APS, Gamma-Seminoprotein, P-30 Antigen, Kallikrein-3, Semenogelase, Seminin, Kallikrein 3, (Prostate Specific Antigen), Prostate Specific Antigen, Prostate-Specific Antigen, EC 3.4.21.77, EC 3.4.21, KLK2A1, and HK3. The sequence of a human KLK3 mRNA can be found, for example, at GenBank Accession GI: 71834852 (NM_001030047.1; SEQ ID NO: 57). The sequence of a human KLK3 polypeptide sequence can be found, for example, at GenBank Accession No. GI: 71834853 (NP_001025218.1; SEQ ID NO: 58). The term “KLK3”, as used herein, refers to a native KLK3 from any vertebrate source, including mammals such as primates (e.g., humans), unless otherwise indicated. The term encompasses full-length, unprocessed KLK3, as well as any form of KLK3 that results from processing in a cell. The term also encompasses naturally occurring variants of KLK3, such as splice variants or allelic variants.

In one embodiment, the invention includes methods and compositions comprising antibodies that bind to KLK3 for use in treating or preventing hypoglycemia, including for example, in a subject having PBH. Thus, in one embodiment, the KLK3 agonist is an activator of KLK3, which may include, e.g., compositions that activate the expression or functional activity of KLK3. Such activators can target KLK3 directly, or can target molecules that mediate KLK3 function. Exemplary activators of KLK3 include, but are not limited to, agonistic anti-KLK3 antibodies (or antigen binding fragments thereof), small molecule activators of KLK3, and/or stimulatory aptamers that specifically bind KLK3.

In one embodiment, the invention provides methods of treating or preventing hypoglycemia in a subject in need thereof by administering an agonist of KLK3 which is an antibody, or an antigen binding fragment thereof, which specifically binds to KLK3 and activates KLK3 activity. In one embodiment, the methods of modulating glucose described herein include administration of a KLK3 protein or nucleic acid encoding KLK3.

### PLAT

In one embodiment of the invention, an activator of PLAT is used in the methods and compositions of the invention. PLAT is also known as Plasminogen Activator, Tissue, TPA, T-Plasminogen Activator, EC 3.4.21.68, Alteplase, Reteplase, T-PA, Tissue Plasminogen Activator (T-PA), Plasminogen Activator, Tissue Type, Tissue-Type Plasminogen Activator, and EC 3.4.21. The sequence of a human PLAT mRNA can be found, for example, at GenBank Accession GI: 132626665 (NM_000930.3; SEQ ID NO: 59). The sequence of a human PLAT polypeptide sequence can be found, for example, at GenBank Accession No. GI: 4505861 (NP_000921.1; SEQ ID NO: 60). The term “PLAT”, as used herein, refers to a native PLAT from any vertebrate source, including mammals such as primates (e.g., humans), unless otherwise indicated. The term encompasses full-length, unprocessed PLAT, as well as any form of PLAT that results from processing in a cell. The term also encompasses naturally occurring variants of PLAT, such as splice variants or allelic variants.

In one embodiment, the invention includes methods and compositions comprising antibodies that bind to PLAT for use in treating or preventing hypoglycemia, including for example, in a subject having PBH. Thus, in one embodiment, the PLAT agonist is an activator of PLAT, which may include, e.g., compositions that activate the expression or functional activity of PLAT. Such activators can target PLAT directly, or can target molecules that mediate PLAT function. Exemplary activators of PLAT include, but are not limited to, agonistic anti-PLAT antibodies (or antigen binding fragments thereof), small molecule activators of PLAT, and/or stimulatory aptamers that specifically bind PLAT.

In one embodiment, the invention provides methods of treating or preventing hypoglycemia in a subject in need thereof by administering an agonist of PLAT which is an antibody, or an antigen binding fragment thereof, which specifically binds to PLAT and activates PLAT activity. In one embodiment, the methods of modulating glucose described herein include administration of a PLAT protein or nucleic acid encoding PLAT.

### 5. Cytokines

In one embodiment, the glucose modulating molecule is a cytokine. An activator or agonist of a cytokine may be used to increase glucose level and treat or prevent hypoglycemia in a subject in need thereof. Examples of cytokines include CCL3L1 and CCL27.

### CCL3L1

In one embodiment of the invention, an activator of CCL3L1 is used in the methods and compositions of the invention. CCL3L1 is also known as Chemokine (C—C Motif) Ligand 3-Like 1, SCYA3L1, Tonsillar Lymphocyte LD78 Beta Protein, G0/G1 Switch Regulatory Protein 19-2, Small Inducible Cytokine A3-Like 1, LD78-Beta(1-70), D1751718, G0S19-2, LD78, Small-Inducible Cytokine A3-Like 1, C—C Motif Chemokine 3-Like 1, CCL3L1 CCL3L3, PAT 464.2, LD78BETA, SCYA3L, MIP1AP, and 464.2. The sequence of a human CCL3L1 mRNA can be found, for example, at GenBank Accession GI: 612149802 (NM_021006.5; SEQ ID NO: 61). The sequence of a human CCL3L1 polypeptide sequence can be found, for example, at GenBank Accession No. GI: 27477072 (NP_066286.1; SEQ ID NO: 62). The term “CCL3L1”, as used herein, refers to a native CCL3L1 from any vertebrate source, including mammals such as primates (e.g., humans), unless otherwise indicated. The term encompasses full-length, unprocessed CCL3L1, as well as any form of CCL3L1 that results from processing in a cell. The term also encompasses naturally occurring variants of CCL3L1, such as splice variants or allelic variants.

In one embodiment, the invention includes methods and compositions comprising antibodies that bind to CCL3L1 for use in treating or preventing hypoglycemia, including for example, in a subject having PBH. Thus, in one embodiment, the CCL3L1 agonist is an activator of CCL3L1, which may include, e.g., compositions that activate the expression or functional activity of CCL3L1. Such activators can target CCL3L1 directly, or can target molecules that mediate CCL3L1 function. Exemplary activators of CCL3L1 include, but are not limited to, agonistic anti-CCL3L1 antibodies (or antigen binding fragments thereof), small molecule activators of CCL3L1, and/or stimulatory aptamers that specifically bind CCL3L1.

In one embodiment, the invention provides methods of treating or preventing hypoglycemia in a subject in need thereof by administering an agonist of CCL3L1 which is an antibody, or an antigen binding fragment thereof, which specifically binds to CCL3L1 and activates CCL3L1 activity. In one embodiment, the methods of modulating glucose described herein include administration of a CCL3L1 protein or nucleic acid encoding CCL3L1.

### CCL27

In one embodiment of the invention, an activator of CCL27 is used in the methods and compositions of the invention. CCL27 is also known as Chemokine (C—C Motif) Ligand 27, CC Chemokine ILC, SCYA27, CTACK, ILC, Small Inducible Cytokine Subfamily A (Cys-Cys), Member 27, Cutaneous T-Cell Attracting Chemokine, Cutaneous T-Cell-Attracting Chemokine, IL-11 R-Alpha-Locus Chemokine, IL-11 Ralpha-Locus Chemokine, Small-Inducible Cytokine A27, Skinkine, ESKINE, C—C Motif Chemokine 27, PESKY, CCL27, CTAK, and ALP. The sequence of a human CCL27 mRNA can be found, for example, at GenBank Accession GI: 686661135 (NM_006664.3; SEQ ID NO: 63). The sequence of a human CCL27 polypeptide sequence can be found, for example, at GenBank Accession No. GI: 5730035 (NP_006655.1; SEQ ID NO: 64). The term “CCL27”, as used herein, refers to a native CCL27 from any vertebrate source, including mammals such as primates (e.g., humans), unless otherwise indicated. The term encompasses full-length, unprocessed CCL27, as well as any form of CCL27 that results from processing in a cell. The term also encompasses naturally occurring variants of CCL27, such as splice variants or allelic variants.

In one embodiment, the invention includes methods and compositions comprising antibodies that bind to CCL27 for use in treating or preventing hypoglycemia, including for example, in a subject having PBH. Thus, in one embodiment, the CCL27 agonist is an activator of CCL27, which may include, e.g., compositions that activate the expression or functional activity of CCL27. Such activators can target CCL27 directly, or can target molecules that mediate CCL27 function. Exemplary activators of CCL27 include, but are not limited to, agonistic anti-CCL27 antibodies (or antigen binding fragments thereof), small molecule activators of CCL27, and/or stimulatory aptamers that specifically bind CCL27.

In one embodiment, the invention provides methods of treating or preventing hypoglycemia in a subject in need thereof by administering an agonist of CCL27 which is an antibody, or an antigen binding fragment thereof, which specifically binds to CCL27 and activates CCL27 activity. In one embodiment, the methods of modulating glucose described herein include administration of a CCL27 protein or nucleic acid encoding CCL27.

### 6. Other Molecules

In one embodiment, the glucose modulating molecule has alternative functions as described above. An activator or agonist of these glucose modulating molecule may be used to increase glucose level and treat or prevent hypoglycemia in a subject in need thereof. Examples of these glucose modulating molecules include CD97, AFM, RTN4R, GNLY, PFD5, MB, GPCS, ARSB and SORCS2.

### CD97

In one embodiment of the invention, an activator of CD97 is used in the methods and compositions of the invention. CD97 is also known as Adhesion G Protein-Coupled Receptor E5, Leukocyte Antigen CD97, CD97 Antigen, Seven Transmembrane Helix Receptor, Seven-Span Transmembrane Protein, CD97 Molecule, ADGRES, Seven-Transmembrane, Heterodimeric Receptor Associated With Inflammation, Heterodimeric Receptor Associated With Inflammation, Seven-Transmembrane, and TM7LN1. The sequence of a human CD97 mRNA can be found, for example, at GenBank Accession GI: 336285467 (NM_001025160.2; SEQ ID NO: 65). The sequence of a human CD97 polypeptide sequence can be found, for example, at GenBank Accession No. GI: 68508955 (NP_001020331.1; SEQ ID NO: 66). The term “CD97”, as used herein, refers to a native CD97 from any vertebrate source, including mammals such as primates (e.g., humans), unless otherwise indicated. The term encompasses full-length, unprocessed CD97, as well as any form of CD97 that results from processing in a cell. The term also encompasses naturally occurring variants of CD97, such as splice variants or allelic variants.

In one embodiment, the invention includes methods and compositions comprising antibodies that bind to CD97 for use in treating or preventing hypoglycemia, including for example, in a subject having PBH. Thus, in one embodiment, the CD97 agonist is an activator of CD97, which may include, e.g., compositions that activate the expression or functional activity of CD97. Such activators can target CD97 directly, or can target molecules that mediate CD97 function. Exemplary activators of CD97 include, but are not limited to, agonistic anti-CD97 antibodies (or antigen binding fragments thereof), small molecule activators of CD97, and/or stimulatory aptamers that specifically bind CD97.

In one embodiment, the invention provides methods of treating or preventing hypoglycemia in a subject in need thereof by administering an agonist of CD97 which is an antibody, or an antigen binding fragment thereof, which specifically binds to CD97 and activates CD97 activity. In one embodiment, the methods of modulating glucose described herein include administration of a CD97 protein or nucleic acid encoding CD97.

### AFM

In one embodiment of the invention, an activator of AFM is used in the methods and compositions of the invention. AFM is also known as Afamin, ALB2, ALBA, Alpha-Albumin, Alpha-Alb, and ALF. The sequence of a human AFM mRNA can be found, for example, at GenBank Accession GI: 27754774 (NM_001133.2; SEQ ID NO: 67). The sequence of a human AFM polypeptide sequence can be found, for example, at GenBank Accession No. GI: 4501987 (NP_001124.1; SEQ ID NO: 68). The term “AFM”, as used herein, refers to a native AFM from any vertebrate source, including mammals such as primates (e.g., humans), unless otherwise indicated. The term encompasses full-length, unprocessed AFM, as well as any form of AFM that results from processing in a cell. The term also encompasses naturally occurring variants of AFM, such as splice variants or allelic variants.

In one embodiment, the invention includes methods and compositions comprising antibodies that bind to AFM for use in treating or preventing hypoglycemia, including for example, in a subject having PBH. Thus, in one embodiment, the AFM agonist is an activator of AFM, which may include, e.g., compositions that activate the expression or functional activity of AFM. Such activators can target AFM directly, or can target molecules that mediate AFM function. Exemplary activators of AFM include, but are not limited to, agonistic anti-AFM antibodies (or antigen binding fragments thereof), small molecule activators of AFM, and/or stimulatory aptamers that specifically bind AFM.

In one embodiment, the invention provides methods of treating or preventing hypoglycemia in a subject in need thereof by administering an agonist of AFM which is an antibody, or an antigen binding fragment thereof, which specifically binds to AFM and activates AFM activity. In one embodiment, the methods of modulating glucose described herein include administration of a AFM protein or nucleic acid encoding AFM.

### RTN4R

In one embodiment of the invention, an activator of RTN4R is used in the methods and compositions of the invention. RTN4R is also known as Reticulon 4 Receptor, NOGOR, Nogo-66 Receptor, Nogo Receptor, NGR, Reticulon-4 Receptor, and UNQ330/PRO526. The sequence of a human RTN4R mRNA can be found, for example, at GenBank Accession GI: 47519383 (NM_023004.5; SEQ ID NO: 69). The sequence of a human RTN4R polypeptide sequence can be found, for example, at GenBank Accession No. GI: 13194201 (NP_075380.1; SEQ ID NO: 70). The term “RTN4R”, as used herein, refers to a native RTN4R from any vertebrate source, including mammals such as primates (e.g., humans), unless otherwise indicated. The term encompasses full-length, unprocessed RTN4R, as well as any form of RTN4R that results from processing in a cell. The term also encompasses naturally occurring variants of RTN4R, such as splice variants or allelic variants.

In one embodiment, the invention includes methods and compositions comprising antibodies that bind to RTN4R for use in treating or preventing hypoglycemia, including for example, in a subject having PBH. Thus, in one embodiment, the RTN4R agonist is an activator of RTN4R, which may include, e.g., compositions that activate the expression or functional activity of RTN4R. Such activators can target RTN4R directly, or can target molecules that mediate RTN4R function. Exemplary activators of RTN4R include, but are not limited to, agonistic anti-RTN4R antibodies (or antigen binding fragments thereof), small molecule activators of RTN4R, and/or stimulatory aptamers that specifically bind RTN4R.

In one embodiment, the invention provides methods of treating or preventing hypoglycemia in a subject in need thereof by administering an agonist of RTN4R which is an antibody, or an antigen binding fragment thereof, which specifically binds to RTN4R and activates RTN4R activity. In one embodiment, the methods of modulating glucose described herein include administration of a RTN4R protein or nucleic acid encoding RTN4R.

### GNLY

In one embodiment of the invention, an activator of GNLY is used in the methods and compositions of the invention. GNLY is also known as Granulysin, TLA519, T-Lymphocyte Activation Gene 519, T-Cell Activation Protein 519, Lymphokine LAG-2, D2S69E, LAG2, NKG5, Lymphocyte-Activation Gene 2, Protein NKG5, LAG-2, and 519. The sequence of a human GNLY mRNA can be found, for example, at GenBank Accession GI: 722829094 (NM_001302758.1; SEQ ID NO: 71). The sequence of a human GNLY polypeptide sequence can be found, for example, at GenBank Accession No. GI: 722829095 (NP_001289687.1; SEQ ID NO: 72). The term “GNLY”, as used herein, refers to a native GNLY from any vertebrate source, including mammals such as primates (e.g., humans), unless otherwise indicated. The term encompasses full-length, unprocessed GNLY, as well as any form of GNLY that results from processing in a cell. The term also encompasses naturally occurring variants of GNLY, such as splice variants or allelic variants.

In one embodiment, the invention includes methods and compositions comprising antibodies that bind to GNLY for use in treating or preventing hypoglycemia, including for example, in a subject having PBH. Thus, in one embodiment, the GNLY agonist is an activator of GNLY, which may include, e.g., compositions that activate the expression or functional activity of GNLY. Such activators can target GNLY directly, or can target molecules that mediate GNLY function. Exemplary activators of GNLY include, but are not limited to, agonistic anti-GNLY antibodies (or antigen binding fragments thereof), small molecule activators of GNLY, and/or stimulatory aptamers that specifically bind GNLY.

In one embodiment, the invention provides methods of treating or preventing hypoglycemia in a subject in need thereof by administering an agonist of GNLY which is an antibody, or an antigen binding fragment thereof, which specifically binds to GNLY and activates GNLY activity. In one embodiment, the methods of modulating glucose described herein include administration of a GNLY protein or nucleic acid encoding GNLY.

### PFD5

In one embodiment of the invention, an activator of PFD5 is used in the methods and compositions of the invention. PFD5 is also known as Prefoldin Subunit 5, MM1, PFDNS, C-Myc-Binding Protein Mm-1, C-Myc Binding Protein, Myc Modulator-1, Myc Modulator 1, Prefoldin 5, and MM-1. The sequence of a human PFD5 mRNA can be found, for example, at GenBank Accession GI: 88999578 (NM_002624.3; SEQ ID NO: 73). The sequence of a human PFD5 polypeptide sequence can be found, for example, at GenBank Accession No. GI: 22202633 (NP_002615.2; SEQ ID NO: 74). The term “PFD5”, as used herein, refers to a native PFD5 from any vertebrate source, including mammals such as primates (e.g., humans), unless otherwise indicated. The term encompasses full-length, unprocessed PFD5, as well as any form of PFD5 that results from processing in a cell. The term also encompasses naturally occurring variants of PFD5, such as splice variants or allelic variants.

In one embodiment, the invention includes methods and compositions comprising antibodies that bind to PFD5 for use in treating or preventing hypoglycemia, including for example, in a subject having PBH. Thus, in one embodiment, the PFD5 agonist is an activator of PFD5, which may include, e.g., compositions that activate the expression or functional activity of PFD5. Such activators can target PFD5 directly, or can target molecules that mediate PFD5 function. Exemplary activators of PFD5 include, but are not limited to, agonistic anti-PFD5 antibodies (or antigen binding fragments thereof), small molecule activators of PFD5, and/or stimulatory aptamers that specifically bind PFD5.

In one embodiment, the invention provides methods of treating or preventing hypoglycemia in a subject in need thereof by administering an agonist of PFD5 which is an antibody, or an antigen binding fragment thereof, which specifically binds to PFD5 and activates PFD5 activity. In one embodiment, the methods of modulating glucose described herein include administration of a PFD5 protein or nucleic acid encoding PFD5.

### MB

In one embodiment of the invention, an activator of MB is used in the methods and compositions of the invention. MB is also known as Myoglobin, Myoglobgin, and PVALB. The sequence of a human MB mRNA can be found, for example, at GenBank Accession GI: 44955876 (NM_005368.2; SEQ ID NO: 75). The sequence of a human MB polypeptide sequence can be found, for example, at GenBank Accession No. GI: 4885477 (NP_005359.1; SEQ ID NO: 76). The term “MB”, as used herein, refers to a native MB from any vertebrate source, including mammals such as primates (e.g., humans), unless otherwise indicated. The term encompasses full-length, unprocessed MB, as well as any form of MB that results from processing in a cell. The term also encompasses naturally occurring variants of MB, such as splice variants or allelic variants.

In one embodiment, the invention includes methods and compositions comprising antibodies that bind to MB for use in treating or preventing hypoglycemia, including for example, in a subject having PBH. Thus, in one embodiment, the MB agonist is an activator of MB, which may include, e.g., compositions that activate the expression or functional activity of MB. Such activators can target MB directly, or can target molecules that mediate MB function. Exemplary activators of MB include, but are not limited to, agonistic anti-MB antibodies (or antigen binding fragments thereof), small molecule activators of MB, and/or stimulatory aptamers that specifically bind MB.

In one embodiment, the invention provides methods of treating or preventing hypoglycemia in a subject in need thereof by administering an agonist of MB which is an antibody, or an antigen binding fragment thereof, which specifically binds to MB and activates MB activity. In one embodiment, the methods of modulating glucose described herein include administration of a MB protein or nucleic acid encoding MB.

### GPC5

In one embodiment of the invention, an activator of GPC5 is used in the methods and compositions of the invention. GPCS is also known as Glypican 5, Glypican Proteoglycan 5, Glypican-5, and BA93M14. The sequence of a human GPC5 mRNA can be found, for example, at GenBank Accession GI: 634743266 (NM_004466.5; SEQ ID NO: 77). The sequence of a human GPC5 polypeptide sequence can be found, for example, at GenBank Accession No. GI: 4758464 (NP_004457.1; SEQ ID NO: 78). The term “GPC5”, as used herein, refers to a native GPC5 from any vertebrate source, including mammals such as primates (e.g., humans), unless otherwise indicated. The term encompasses full-length, unprocessed GPC5, as well as any form of GPC5 that results from processing in a cell. The term also encompasses naturally occurring variants of GPC5, such as splice variants or allelic variants.

In one embodiment, the invention includes methods and compositions comprising antibodies that bind to GPC5 for use in treating or preventing hypoglycemia, including for example, in a subject having PBH. Thus, in one embodiment, the GPC5 agonist is an activator of GPC5, which may include, e.g., compositions that activate the expression or functional activity of GPC5. Such activators can target GPC5 directly, or can target molecules that mediate GPC5 function. Exemplary activators of GPC5 include, but are not limited to, agonistic anti-GPC5 antibodies (or antigen binding fragments thereof), small molecule activators of GPC5, and/or stimulatory aptamers that specifically bind GPC5.

In one embodiment, the invention provides methods of treating or preventing hypoglycemia in a subject in need thereof by administering an agonist of GPC5 which is an antibody, or an antigen binding fragment thereof, which specifically binds to GPC5 and activates GPC5 activity. In one embodiment, the methods of modulating glucose described herein include administration of a GPC5 protein or nucleic acid encoding GPC5.

### ARSB

In one embodiment of the invention, an activator of ARSB is used in the methods and compositions of the invention. ARSB is also known as Arylsulfatase B, N-Acetylgalactosamine-4-Sulfatase, EC 3.1.6.12, MPS6, ASB, and G4S. The sequence of a human ARSB mRNA can be found, for example, at GenBank Accession GI: 158634485 (NM_000046.3; SEQ ID NO: 79). The sequence of a human ARSB polypeptide sequence can be found, for example, at GenBank Accession No. GI: 38569405 (NP_000037.2; SEQ ID NO: 80). The term “ARSB”, as used herein, refers to a native ARSB from any vertebrate source, including mammals such as primates (e.g., humans), unless otherwise indicated. The term encompasses full-length, unprocessed ARSB, as well as any form of ARSB that results from processing in a cell. The term also encompasses naturally occurring variants of ARSB, such as splice variants or allelic variants.

In one embodiment, the invention includes methods and compositions comprising antibodies that bind to ARSB for use in treating or preventing hypoglycemia, including for example, in a subject having PBH. Thus, in one embodiment, the ARSB agonist is an activator of ARSB, which may include, e.g., compositions that activate the expression or functional activity of ARSB. Such activators can target ARSB directly, or can target molecules that mediate ARSB function. Exemplary activators of ARSB include, but are not limited to, agonistic anti-ARSB antibodies (or antigen binding fragments thereof), small molecule activators of ARSB, and/or stimulatory aptamers that specifically bind ARSB.

In one embodiment, the invention provides methods of treating or preventing hypoglycemia in a subject in need thereof by administering an agonist of ARSB which is an antibody, or an antigen binding fragment thereof, which specifically binds to ARSB and activates ARSB activity. In one embodiment, the methods of modulating glucose described herein include administration of a ARSB protein or nucleic acid encoding ARSB.

### SORCS2

In one embodiment of the invention, an activator of SORCS2 is used in the methods and compositions of the invention. SORCS2 is also known as Sortilin-Related VPS10 Domain Containing Receptor 2 and KIAA132. The sequence of a human SORCS2 mRNA can be found, for example, at GenBank Accession GI: 170014688 (NM_020777.2; SEQ ID NO: 81). The sequence of a human SORCS2 polypeptide sequence can be found, for example, at GenBank Accession No. GI: 170014689 (NP_065828.2; SEQ ID NO: 82). The term “SORCS2”, as used herein, refers to a native SORCS2 from any vertebrate source, including mammals such as primates (e.g., humans), unless otherwise indicated. The term encompasses full-length, unprocessed SORCS2, as well as any form of SORCS2 that results from processing in a cell. The term also encompasses naturally occurring variants of SORCS2, such as splice variants or allelic variants.

In one embodiment, the invention includes methods and compositions comprising antibodies that bind to SORCS2 for use in treating or preventing hypoglycemia, including for example, in a subject having PBH. Thus, in one embodiment, the SORCS2 agonist is an activator of SORCS2, which may include, e.g., compositions that activate the expression or functional activity of SORCS2. Such activators can target SORCS2 directly, or can target molecules that mediate SORCS2 function. Exemplary activators of SORCS2 include, but are not limited to, agonistic anti-SORCS2 antibodies (or antigen binding fragments thereof), small molecule activators of SORCS2, and/or stimulatory aptamers that specifically bind SORCS2.

In one embodiment, the invention provides methods of treating or preventing hypoglycemia in a subject in need thereof by administering an agonist of SORCS2 which is an antibody, or an antigen binding fragment thereof, which specifically binds to SORCS2 and activates SORCS2 activity. In one embodiment, the methods of modulating glucose described herein include administration of a SORCS2 protein or nucleic acid encoding SORCS2.

### 7. Agonists of Glucose Modulating Molecules

In one embodiment, the invention includes methods of administering agonists that will increase activity levels of certain glucose modulating molecules associated with hypoglycemia. Thus, by increasing or activating the activity of these glucose modulating molecules, glucose levels in a subject, e.g., a human subject, will increase.

### Agonistic Antibodies

The invention contemplates methods and compositions comprising antibodies that bind to a glucose modulating molecule, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB or SORCS2, and/or a receptor specific for the glucose modulating molecule, or antigen-binding fragments thereof, for use in the methods described herein.

In one embodiment, the antibody, or antigen-binding fragment thereof, is an agonistic antibody or antigen-binding fragment thereof, specific for the glucose modulating molecule and/or the receptor for the glucose modulating molecule. In one embodiment, the agonistic antibody or antigen-binding fragment thereof increases the activity of the glucose modulating molecule. In another embodiment, the agonistic antibodies or antigen-binding fragments thereof, are chimeric, humanized or fully human antibodies, or antigen-binding fragments thereof.

In some embodiments, the agonistic antibody, or antigen binding fragment thereof, for the glucose modulating molecule, increases the blood glucose level or reduces the symptoms of hypoglycemia.

Antibodies specific for the glucose modulating molecules and/or the receptor for the glucose modulating molecules may be identified, screened for (e.g, using phage display), or characterized for their physical/chemical properties and/or biological activities by various assays known in the art (see, for example, Antibodies: A Laboratory Manual, Second edition, Greenfield, ed. 2014), Assays, for example, described in the Examples may be used to identify antibodies having advantageous properties, such as the ability to increase blood glucose level. In one aspect, an antibody for HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB or SORCS2 is tested for its antigen binding activity, e.g., by known methods such as ELISA, Western blot, etc.

Following identification of the antigen of the antibody e.g., ability to bind the glucose modulating molecule, or a receptor thereof, the activity of the antibody may be tested. In one aspect, assays are provided for identifying antibodies specific for the glucose modulating moelcules, thereof having antagonist activity. For example, biological activity may include the ability to activate signal transduction of particular pathways which can be measured, e.g., by determining levels of FGF19-induced downregulation of cyp7.alpha.1 was assessed using hepatocellular carcinoma HEP3B cells (Schlessinger, Science 306:1506-1507 (2004))

Following screening and sequencing, antibodies may he produced using recombinant methods and compositions, e.g., as described in U.S. Pat. No. 4,816,567, incorporated by reference herein. An isolated nucleic acid encoding the antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell).

For recombinant production of an antibody specific for the glucose modulating moelcules, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived, from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Small Molecule Activators

In one embodiment, the agonists for the glucose modulating molecules for use in any of the methods described herein are test compounds. Test compounds that act as an inhibitor for the glucose modulating molecule, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2, and/or a receptor specific for the glucose modulating molecule, can be identified through screening assays. The test compounds can be, e.g., natural products or members of a combinatorial chemistry library.

In some embodiments, the test compounds that act as agonists for the glucose modulating molecules are small molecules. In some embodiments, the agonists for the glucose modulating molecules increase the expression and/or activity of the glucose modulating molecule and/or the receptor specific for the glucose modulating molecule. In some embodiments, the small molecule binds to the glucose modulating molecule. In some embodiments, the small molecule binds to a receptor for the glucose modulating molecule.

In some embodiments, the test compounds are initially members of a library, e.g., an inorganic or organic chemical library, peptide library, oligonucleotide library, or mixed-molecule library. In some embodiments, the methods include screening small molecules, e.g., natural products or members of a combinatorial chemistry library. These methods can also be used, for example, to screen a library of proteins or fragments thereof, e.g., proteins that are expressed in liver or pancreatic cells.

A given library can comprise a set of structurally related or unrelated test compounds. Preferably, a set of diverse molecules should be used to cover a variety of functions such as charge, aromaticity, hydrogen bonding, flexibility, size, length of side chain, hydrophobicity, and rigidity. Combinatorial techniques suitable for creating libraries are known in the art, e.g., methods for synthesizing libraries of small molecules, e.g., as exemplified by Obrecht and Villalgordo, Solid-Supported Combinatorial and Parallel Synthesis of Small-Molecular-Weight Compound Libraries, Pergamon-Elsevier Science Limited (1998). Such methods include the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of libraries, including small molecule libraries, are commercially available.

In some embodiments, the test compounds are peptide or peptidomimetic molecules, e.g., peptide analogs including peptides comprising non-naturally occurring amino acids or having non-peptide linkages; peptidomimetics (e.g., peptoid oligomers, e.g., peptoid amide or ester analogues, .theta.-peptides, D-peptides, L-peptides, oligourea or oligocarbamate); small peptides (e.g., pentapeptides, hexapeptides, heptapeptides, octapeptides, nonapeptides, decapeptides, or larger, e.g., 20-mers or more); cyclic peptides; other non-natural or unnatural peptide-like structures; and inorganic molecules (e.g., heterocyclic ring molecules). In some embodiments, the test compounds are nucleic acids, e.g., DNA or RNA oligonucleotides.

In some embodiments, test compounds and libraries thereof can be obtained by systematically altering the structure of a first test compound. Taking a small molecule as an example, e.g., a first small molecule is selected that is, e.g., structurally similar to a known phosphorylation or protein recognition site. For example, in one embodiment, a general library of small molecules is screened, e.g., using the methods described herein, to select a first test small molecule. Using methods known in the art, the structure of that small molecule is identified if necessary and correlated to a resulting biological activity, e.g., by a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds.

In some embodiments, test compounds identified as “hits” (e.g., test compounds that demonstrate activity in a method described herein) in a first screen are selected and optimized by being systematically altered, e.g., using rational design, to optimize binding affinity, avidity, specificity, or other parameter. Such potentially optimized structures can also be screened using the methods described herein. Thus, in one embodiment, the invention includes screening a first library of test compounds using a method described herein, identifying one or more hits in that library, subjecting those hits to systematic structural alteration to create one or more second generation compounds structurally related to the hit, and screening the second generation compound. Additional rounds of optimization can be used to identify a test compound with a desirable therapeutic profile.

Test compounds identified as hits can be considered candidate therapeutic compounds, useful in treating disorders described herein. Thus, the invention also includes compounds identified as “hits” by a method described herein, and methods for their administration and use in the treatment, prevention, or delay of development or progression of a disease described herein.

### Mimetics

Variants of the glucose modulating molecule, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2, and/or a receptor specific for the glucose modulating molecule, can be identified by screening combinatorial libraries of mutants.

In some embodiments, the agonists specific for the glucose modulating molecules are variants of the glucose modulating molecule. In some embodiments, the variants for the glucose modulating molecules increase the expression and/or activity of the glucose modulating molecule and/or the receptor specific for the glucose modulating molecule.

In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the marker proteins from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

Thus, in a further embodiment, the methods of the invention also may be practiced using a mimetic of an agonist of the glucose modulating molecules.

### II.C. Pharmaceutical Formulations

Pharmaceutical formulations comprising antagonists of the glucose modulating molecules FGF19, IGFBP1, ADIPOQ, GCG, SHBG, CXCL3, CXCL2, TNFRSF17, AMICA1, TFF3, EFNB3 and LSAMP, and/or agonists of the glucose modulating molecules HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPCS, ARSB and SORCS2, of the present invention may be prepared for storage by mixing the protein or nucleic acid having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™, Pluronics™ or polyethylene glycol (PEG).

The formulation herein may also contain more than one active compound as necessary for the particular indication being treated (e.g., a disease that would benefit from glucose control, a disease that would benefit from weight control, a disease that would benefit from appetite control), preferably those with complementary activities that do not adversely affect each other. Such molecules are suitably present in combination in amounts that are effective for the purpose intended.

The active ingredients may also be packaged in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Generally, the ingredients of compositions are supplied either separately or mixed together in unit dosage form, for example, as a dry lyophilized powder or water free concentrate in a hermetically sealed container such as an ampoule or sachet indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration. In an alternative embodiment, one or more of the pharmaceutical compositions of the invention is supplied in liquid form in a hermetically sealed container indicating the quantity and concentration of the agent.

The active agent can be incorporated into a pharmaceutical composition suitable for parenteral administration, typically prepared as an injectable solution. The injectable solution can be composed of either a liquid or lyophilized dosage form in a flint or amber vial, ampule or pre-filled syringe. The liquid or lyophilized dosage may further comprise a buffer (e.g., L-histidine, sodium succinate, sodium citrate, sodium phosphate or potassium phosphate, sodium chloride), a cryoprotectant (e.g., sucrose trehalose or lactose, a bulking agent (e.g., mannitol), a stabilizer (e.g., L-Methionine, glycine, arginine), an adjuvant (hyaluronidase).

The compositions of this invention may be in a variety of forms. These include, for example, liquid, semi-solid and solid dosage forms, such as liquid solutions (e.g., injectable and infusible solutions), microemulsion, dispersions, liposomes or suspensions, tablets, pills, powders, liposomes and suppositories. The preferred form depends on the intended mode of administration and therapeutic application. Typical modes of administration include parenteral (e.g., intravenous, subcutaneous, intraperitoneal, intramuscular) injection or oral administration. In a preferred embodiment, the antagonist or agonist of a glucose modulating molecule is administered by injection. In another embodiment, the injection is subcutaneous. In a particular embodiment, the administration is into adipose tissue

Pharmaceutical compositions comprising an agent described herein may be formulated for administration to a particular tissue. For example, in certain embodiments, it may be desirable to administer the agent into adipose tissue, either in a diffuse fashion or targeted to a site (e.g., subcutaneous adipose tissue).

In another aspect, the invention provides pharmaceutical compositions that utilize cells in various methods for treatment of diseases that would benefit from glucose control, weight control and or appetite control. Certain embodiments encompass pharmaceutical compositions comprising live cells. The pharmaceutical composition may further comprise other active agents, such as anti-inflammatory agents, anti-apoptotic agents, antioxidants or growth factors.

### II.D. Therapeutic Methods of the Invention

In certain embodiments, the present invention provides methods of treating or reducing the symptoms of hypoglycemia in a subject in need thereof, e.g., increasing the blood glucose level. In one embodiment, an antagonist of a glucose modulating molecule described herein is administered to the subject in need thereof. In one embodiment, the glucose modulating molecule is selected from the group consisting of FGF19, IGFBP1, ADIPOQ, GCG, SHBG, CXCL3, CXCL2, TNFRSF17, AMICA1, TFF3, EFNB3 and LSAMP. In one embodiment, an agonist of a glucose modulating molecule described herein is administered to the subject in need thereof. In one embodiment, the glucose modulating molecule is selected from the group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2.

In some embodiments, an antagonist for FGF19 is administered to the subject in need thereof. In another embodiment, the FGF19 antagonist is an inhibitor of FGF19, which may include, e.g., compositions that inhibit the expression or functional activity of FGF19, as described herein. Such inhibitors can target FGF19 directly, or can target receptors which bind FGF19 and consequently mediate FGF19 function. Exemplary inhibitors of FGF19 can include, but are not limited to, antagonistic anti-FGF19 antibodies (or antigen binding fragments thereof), soluble forms of FGF19 receptors, small molecule inhibitors specific for FGF19, inhibitory polynucleotides, e.g., anti-sense oligonucleotides, siRNA or shRNA specific for FGF19, and/or inhibitory aptamers that specifically bind FGF19.

In some embodiments, an antagonist for IGFBP1 is administered to the subject in need thereof. In another embodiment, the IGFBP1 antagonist is an inhibitor of IGFBP1, which may include, e.g., compositions that inhibit the expression or functional activity of IGFBP1, as described herein. Such inhibitors can target IGFBP1 directly, or can target receptors which bind IGFBP1 and consequently mediate IGFBP1 function. Exemplary inhibitors of IGFBP1 can include, but are not limited to, antagonistic anti-IGFBP1 antibodies (or antigen binding fragments thereof), soluble forms of an IGFBP1 receptors, small molecule inhibitors specific for IGFBP1, inhibitory polynucleotides, e.g., anti-sense oligonucleotides, siRNA or shRNA specific for IGFBP1, and/or inhibitory aptamers that specifically bind IGFBP1.

In some embodiments, an antagonist for ADIPOQ is administered to the subject in need thereof. In another embodiment, the ADIPOQ antagonist is an inhibitor of ADIPOQ, which may include, e.g., compositions that inhibit the expression or functional activity of ADIPOQ, as described herein. Such inhibitors can target ADIPOQ directly, or can target receptors which bind ADIPOQ and consequently mediate ADIPOQ function. Exemplary inhibitors of ADIPOQ can include, but are not limited to, antagonistic anti-ADIPOQ antibodies (or antigen binding fragments thereof), soluble forms of an ADIPOQ receptors, small molecule inhibitors specific for ADIPOQ, inhibitory polynucleotides, e.g., anti-sense oligonucleotides, siRNA or shRNA specific for ADIPOQ, and/or inhibitory aptamers that specifically bind ADIPOQ.

In some embodiments, an antagonist for GCG is administered to the subject in need thereof. In another embodiment, the GCG antagonist is an inhibitor of GCG, which may include, e.g., compositions that inhibit the expression or functional activity of GCG, as described herein. Such inhibitors can target GCG directly, or can target receptors which bind GCG and consequently mediate GCG function. Exemplary inhibitors of GCG can include, but are not limited to, antagonistic anti-GCG antibodies (or antigen binding fragments thereof), soluble forms of an GCG receptors, small molecule inhibitors specific for GCG, inhibitory polynucleotides, e.g., anti-sense oligonucleotides, siRNA or shRNA specific for GCG, and/or inhibitory aptamers that specifically bind GCG.

In some embodiments, an antagonist for SHBG is administered to the subject in need thereof. In another embodiment, the SHBG antagonist is an inhibitor of SHBG, which may include, e.g., compositions that inhibit the expression or functional activity of SHBG, as described herein. Such inhibitors can target SHBG directly, or can target receptors which bind SHBG and consequently mediate SHBG function. Exemplary inhibitors of SHBG can include, but are not limited to, antagonistic anti-SHBG antibodies (or antigen binding fragments thereof), soluble forms of an SHBG receptors, small molecule inhibitors specific for SHBG, inhibitory polynucleotides, e.g., anti-sense oligonucleotides, siRNA or shRNA specific for SHBG, and/or inhibitory aptamers that specifically bind SHBG.

In some embodiments, an antagonist for CXCL3 is administered to the subject in need thereof. In another embodiment, the CXCL3 antagonist is an inhibitor of CXCL3, which may include, e.g., compositions that inhibit the expression or functional activity of CXCL3, as described herein. Such inhibitors can target CXCL3 directly, or can target receptors which bind CXCL3 and consequently mediate CXCL3 function. Exemplary inhibitors of CXCL3 can include, but are not limited to, antagonistic anti-CXCL3 antibodies (or antigen binding fragments thereof), soluble forms of an CXCL3 receptors, small molecule inhibitors specific for CXCL3, inhibitory polynucleotides, e.g., anti-sense oligonucleotides, siRNA or shRNA specific for CXCL3, and/or inhibitory aptamers that specifically bind CXCL3.

In some embodiments, an antagonist for CXCL2 is administered to the subject in need thereof. In another embodiment, the CXCL2 antagonist is an inhibitor of CXCL2, which may include, e.g., compositions that inhibit the expression or functional activity of CXCL2, as described herein. Such inhibitors can target CXCL2 directly, or can target receptors which bind CXCL2 and consequently mediate CXCL2 function. Exemplary inhibitors of CXCL2 can include, but are not limited to, antagonistic anti-CXCL2 antibodies (or antigen binding fragments thereof), soluble forms of an CXCL2 receptors, small molecule inhibitors specific for CXCL2, inhibitory polynucleotides, e.g., anti-sense oligonucleotides, siRNA or shRNA specific for CXCL2, and/or inhibitory aptamers that specifically bind CXCL2.

In some embodiments, an antagonist for TNFRSF17 is administered to the subject in need thereof. In another embodiment, the TNFRSF17 antagonist is an inhibitor of TNFRSF17, which may include, e.g., compositions that inhibit the expression or functional activity of TNFRSF17, as described herein. Such inhibitors can target TNFRSF17 directly, or can target receptors which bind TNFRSF17 and consequently mediate TNFRSF17 function. Exemplary inhibitors of TNFRSF17 can include, but are not limited to, antagonistic anti-TNFRSF17 antibodies (or antigen binding fragments thereof), soluble forms of an TNFRSF17 receptors, small molecule inhibitors specific for TNFRSF17, inhibitory polynucleotides, e.g., anti-sense oligonucleotides, siRNA or shRNA specific for TNFRSF17, and/or inhibitory aptamers that specifically bind TNFRSF17.

In some embodiments, an antagonist for AMICA1 is administered to the subject in need thereof. In another embodiment, the AMICA1 antagonist is an inhibitor of AMICA1, which may include, e.g., compositions that inhibit the expression or functional activity of AMICA1, as described herein. Such inhibitors can target AMICA1 directly, or can target receptors which bind AMICA1 and consequently mediate AMICA1 function. Exemplary inhibitors of AMICA1 can include, but are not limited to, antagonistic anti-AMICA1 antibodies (or antigen binding fragments thereof), soluble forms of an AMICA1 receptors, small molecule inhibitors specific for AMICA1, inhibitory polynucleotides, e.g., anti-sense oligonucleotides, siRNA or shRNA specific for AMICA1, and/or inhibitory aptamers that specifically bind AMICA1.

In some embodiments, an antagonist for TFF3 is administered to the subject in need thereof. In another embodiment, the TFF3 antagonist is an inhibitor of TFF3, which may include, e.g., compositions that inhibit the expression or functional activity of TFF3, as described herein. Such inhibitors can target TFF3 directly, or can target receptors which bind TFF3 and consequently mediate TFF3 function. Exemplary inhibitors of TFF3 can include, but are not limited to, antagonistic anti-TFF3 antibodies (or antigen binding fragments thereof), soluble forms of an TFF3 receptors, small molecule inhibitors specific for TFF3, inhibitory polynucleotides, e.g., anti-sense oligonucleotides, siRNA or shRNA specific for TFF3, and/or inhibitory aptamers that specifically bind TFF3.

In some embodiments, an antagonist for EFNB3 is administered to the subject in need thereof. In another embodiment, the EFNB3 antagonist is an inhibitor of EFNB3, which may include, e.g., compositions that inhibit the expression or functional activity of EFNB3, as described herein. Such inhibitors can target EFNB3 directly, or can target receptors which bind EFNB3 and consequently mediate EFNB3 function. Exemplary inhibitors of EFNB3 can include, but are not limited to, antagonistic anti-EFNB3 antibodies (or antigen binding fragments thereof), soluble forms of an EFNB3 receptors, small molecule inhibitors specific for EFNB3, inhibitory polynucleotides, e.g., anti-sense oligonucleotides, siRNA or shRNA specific for EFNB3, and/or inhibitory aptamers that specifically bind EFNB 3.

In some embodiments, an antagonist for LSAMP is administered to the subject in need thereof. In another embodiment, the LSAMP antagonist is an inhibitor of LSAMP, which may include, e.g., compositions that inhibit the expression or functional activity of LSAMP, as described herein. Such inhibitors can target LSAMP directly, or can target receptors which bind LSAMP and consequently mediate LSAMP function. Exemplary inhibitors of LSAMP can include, but are not limited to, antagonistic anti-LSAMP antibodies (or antigen binding fragments thereof), soluble forms of an LSAMP receptors, small molecule inhibitors specific for LSAMP, inhibitory polynucleotides, e.g., anti-sense oligonucleotides, siRNA or shRNA specific for LSAMP, and/or inhibitory aptamers that specifically bind LSAMP.

The present invention also provides methods of treating or reducing the symptoms of hypoglycemia in a subject in need thereof, e.g., increasing the blood glucose level, by administering an agonist of a glucose modulating molecule to the subject in need thereof. In some embodiment, the glucose modulating molecule is selected from the group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2.

In one embodiment, the agonist of the glucose modulating molecule is an activator of the glucose modulating molecule, which may include, e.g., compositions that increase the expression or functional activity of the glucose modulating molecule. Such activators can target the glucose modulating molecule directly, or can target receptors which bind the glucose modulating molecule and consequently mediate the glucose modulating molecule function. Exemplary activators of the glucose modulating molecule can include, but are not limited to, agonistic antibodies (or antigen binding fragments thereof) specific for the glucose modulating molecule, small molecule activators specific for the glucose modulating molecule, small molecule activators specific for the receptor of the glucose modulating molecule, and/or stimulatory aptamers that specifically bind the glucose modulating molecule.

In one embodiment, the agonist of HGFAC is an activator of HGFAC, which may include, e.g., compositions that increase the expression or functional activity of HGFAC. Such activators can target HGFAC directly, or can target receptors which bind HGFAC and consequently mediate the function of HGFAC. Exemplary activators of HGFAC can include, but are not limited to, agonistic antibodies (or antigen binding fragments thereof) specific for HGFAC, small molecule activators specific for HGFAC, small molecule activators specific for the receptor of HGFAC, and/or stimulatory aptamers that specifically bind HGFAC.

In one embodiment, the agonist of BMPR2 is an activator of BMPR2, which may include, e.g., compositions that increase the expression or functional activity of BMPR2. Such activators can target BMPR2 directly, or can target receptors which bind BMPR2 and consequently mediate the function of BMPR2. Exemplary activators of BMPR2 can include, but are not limited to, agonistic antibodies (or antigen binding fragments thereof) specific for BMPR2, small molecule activators specific for BMPR2, small molecule activators specific for the receptor of BMPR2, and/or stimulatory aptamers that specifically bind BMPR2.

In one embodiment, the agonist of GDF11 is an activator of GDF11, which may include, e.g., compositions that increase the expression or functional activity of GDF11. Such activators can target GDF11 directly, or can target receptors which bind GDF11 and consequently mediate the function of GDF11. Exemplary activators of GDF11 can include, but are not limited to, agonistic antibodies (or antigen binding fragments thereof) specific for GDF11, small molecule activators specific for GDF11, small molecule activators specific for the receptor of GDF11, and/or stimulatory aptamers that specifically bind GDF11.

In one embodiment, the agonist of IGFBP7 is an activator of IGFBP7, which may include, e.g., compositions that increase the expression or functional activity of IGFBP7. Such activators can target IGFBP7 directly, or can target receptors which bind IGFBP7 and consequently mediate the function of IGFBP7. Exemplary activators of IGFBP7 can include, but are not limited to, agonistic antibodies (or antigen binding fragments thereof) specific for IGFBP7, small molecule activators specific for IGFBP7, small molecule activators specific for the receptor of IGFBP7, and/or stimulatory aptamers that specifically bind IGFBP7.

In one embodiment, the agonist of IGFBP6 is an activator of IGFBP6, which may include, e.g., compositions that increase the expression or functional activity of IGFBP6. Such activators can target IGFBP6 directly, or can target receptors which bind IGFBP6 and consequently mediate the function of IGFBP6. Exemplary activators of IGFBP6 can include, but are not limited to, agonistic antibodies (or antigen binding fragments thereof) specific for IGFBP6, small molecule activators specific for IGFBP6, small molecule activators specific for the receptor of IGFBP6, and/or stimulatory aptamers that specifically bind IGFBP6.

In one embodiment, the agonist of APOE is an activator of APOE, which may include, e.g., compositions that increase the expression or functional activity of APOE. Such activators can target APOE directly, or can target receptors which bind APOE and consequently mediate the function of APOE. Exemplary activators of APOE can include, but are not limited to, agonistic antibodies (or antigen binding fragments thereof) specific for APOE, small molecule activators specific for APOE, small molecule activators specific for the receptor of APOE, and/or stimulatory aptamers that specifically bind APOE.

In one embodiment, the agonist of PLA2G7 is an activator of PLA2G7, which may include, e.g., compositions that increase the expression or functional activity of PLA2G7. Such activators can target PLA2G7 directly, or can target receptors which bind PLA2G7 and consequently mediate the function of PLA2G7. Exemplary activators of PLA2G7 can include, but are not limited to, agonistic antibodies (or antigen binding fragments thereof) specific for PLA2G7, small molecule activators specific for PLA2G7, small molecule activators specific for the receptor of PLA2G7, and/or stimulatory aptamers that specifically bind PLA2G7.

In one embodiment, the agonist of CDK2 is an activator of CDK2, which may include, e.g., compositions that increase the expression or functional activity of CDK2. Such activators can target CDK2 directly, or can target receptors which bind CDK2 and consequently mediate the function of CDK2. Exemplary activators of CDK2 can include, but are not limited to, agonistic antibodies (or antigen binding fragments thereof) specific for CDK2, small molecule activators specific for CDK2, small molecule activators specific for the receptor of CDK2, and/or stimulatory aptamers that specifically bind CDK2.

In one embodiment, the agonist of CCNA2 is an activator of CCNA2, which may include, e.g., compositions that increase the expression or functional activity of CCNA2. Such activators can target CCNA2 directly, or can target receptors which bind CCNA2 and consequently mediate the function of CCNA2. Exemplary activators of CCNA2 can include, but are not limited to, agonistic antibodies (or antigen binding fragments thereof) specific for CCNA2, small molecule activators specific for CCNA2, small molecule activators specific for the receptor of CCNA2, and/or stimulatory aptamers that specifically bind CCNA2.

In one embodiment, the agonist of MAPKAPK3 is an activator of MAPKAPK3, which may include, e.g., compositions that increase the expression or functional activity of MAPKAPK3. Such activators can target MAPKAPK3 directly, or can target receptors which bind MAPKAPK3 and consequently mediate the function of MAPKAPK3. Exemplary activators of MAPKAPK3 can include, but are not limited to, agonistic antibodies (or antigen binding fragments thereof) specific for MAPKAPK3, small molecule activators specific for MAPKAPK3, small molecule activators specific for the receptor of MAPKAPK3, and/or stimulatory aptamers that specifically bind MAPKAPK3.

In one embodiment, the agonist of KLK3 is an activator of KLK3, which may include, e.g., compositions that increase the expression or functional activity of KLK3. Such activators can target KLK3 directly, or can target receptors which bind KLK3 and consequently mediate the function of KLK3. Exemplary activators of KLK3 can include, but are not limited to, agonistic antibodies (or antigen binding fragments thereof) specific for KLK3, small molecule activators specific for KLK3, small molecule activators specific for the receptor of KLK3, and/or stimulatory aptamers that specifically bind KLK3.

In one embodiment, the agonist of PLAT is an activator of PLAT, which may include, e.g., compositions that increase the expression or functional activity of PLAT. Such activators can target PLAT directly, or can target receptors which bind PLAT and consequently mediate the function of PLAT. Exemplary activators of PLAT can include, but are not limited to, agonistic antibodies (or antigen binding fragments thereof) specific for PLAT, small molecule activators specific for PLAT, small molecule activators specific for the receptor of PLAT, and/or stimulatory aptamers that specifically bind PLAT.

In one embodiment, the agonist of CCL3L1 is an activator of CCL3L1, which may include, e.g., compositions that increase the expression or functional activity of CCL3L1. Such activators can target CCL3L1 directly, or can target receptors which bind CCL3L1 and consequently mediate the function of CCL3L1. Exemplary activators of CCL3L1 can include, but are not limited to, agonistic antibodies (or antigen binding fragments thereof) specific for CCL3L1, small molecule activators specific for CCL3L1, small molecule activators specific for the receptor of CCL3L1, and/or stimulatory aptamers that specifically bind CCL3L1.

In one embodiment, the agonist of CCL27 is an activator of CCL27, which may include, e.g., compositions that increase the expression or functional activity of CCL27. Such activators can target CCL27 directly, or can target receptors which bind CCL27 and consequently mediate the function of CCL27. Exemplary activators of CCL27 can include, but are not limited to, agonistic antibodies (or antigen binding fragments thereof) specific for CCL27, small molecule activators specific for CCL27, small molecule activators specific for the receptor of CCL27, and/or stimulatory aptamers that specifically bind CCL27.

In one embodiment, the agonist of CD97 is an activator of CD97, which may include, e.g., compositions that increase the expression or functional activity of CD97. Such activators can target CD97 directly, or can target receptors which bind CD97 and consequently mediate the function of CD97. Exemplary activators of CD97 can include, but are not limited to, agonistic antibodies (or antigen binding fragments thereof) specific for CD97, small molecule activators specific for CD97, small molecule activators specific for the receptor of CD97, and/or stimulatory aptamers that specifically bind CD97.

In one embodiment, the agonist of AFM is an activator of AFM, which may include, e.g., compositions that increase the expression or functional activity of AFM. Such activators can target AFM directly, or can target receptors which bind AFM and consequently mediate the function of AFM. Exemplary activators of AFM can include, but are not limited to, agonistic antibodies (or antigen binding fragments thereof) specific for AFM, small molecule activators specific for AFM, small molecule activators specific for the receptor of AFM, and/or stimulatory aptamers that specifically bind AFM.

In one embodiment, the agonist of RTN4R is an activator of RTN4R, which may include, e.g., compositions that increase the expression or functional activity of RTN4R. Such activators can target RTN4R directly, or can target receptors which bind RTN4R and consequently mediate the function of RTN4R. Exemplary activators of RTN4R can include, but are not limited to, agonistic antibodies (or antigen binding fragments thereof) specific for RTN4R, small molecule activators specific for RTN4R, small molecule activators specific for the receptor of RTN4R, and/or stimulatory aptamers that specifically bind RTN4R.

In one embodiment, the agonist of GNLY is an activator of GNLY, which may include, e.g., compositions that increase the expression or functional activity of GNLY. Such activators can target GNLY directly, or can target receptors which bind GNLY and consequently mediate the function of GNLY. Exemplary activators of GNLY can include, but are not limited to, agonistic antibodies (or antigen binding fragments thereof) specific for GNLY, small molecule activators specific for GNLY, small molecule activators specific for the receptor of GNLY, and/or stimulatory aptamers that specifically bind GNLY.

In one embodiment, the agonist of PFD5 is an activator of PFD5, which may include, e.g., compositions that increase the expression or functional activity of PFD5. Such activators can target PFD5 directly, or can target receptors which bind PFD5 and consequently mediate the function of PFD5. Exemplary activators of PFD5 can include, but are not limited to, agonistic antibodies (or antigen binding fragments thereof) specific for PFD5, small molecule activators specific for PFD5, small molecule activators specific for the receptor of PFD5, and/or stimulatory aptamers that specifically bind PFD5.

In one embodiment, the agonist of MB is an activator of MB, which may include, e.g., compositions that increase the expression or functional activity of MB. Such activators can target MB directly, or can target receptors which bind MB and consequently mediate the function of MB. Exemplary activators of MB can include, but are not limited to, agonistic antibodies (or antigen binding fragments thereof) specific for MB, small molecule activators specific for MB, small molecule activators specific for the receptor of MB, and/or stimulatory aptamers that specifically bind MB.

In one embodiment, the agonist of GPC5 is an activator of GPC5, which may include, e.g., compositions that increase the expression or functional activity of GPC5. Such activators can target GPC5 directly, or can target receptors which bind GPC5 and consequently mediate the function of GPC5. Exemplary activators of GPC5 can include, but are not limited to, agonistic antibodies (or antigen binding fragments thereof) specific for GPC5, small molecule activators specific for GPC5, small molecule activators specific for the receptor of GPC5, and/or stimulatory aptamers that specifically bind GPC5.

In one embodiment, the agonist of ARSB is an activator of ARSB, which may include, e.g., compositions that increase the expression or functional activity of ARSB. Such activators can target ARSB directly, or can target receptors which bind ARSB and consequently mediate the function of ARSB. Exemplary activators of ARSB can include, but are not limited to, agonistic antibodies (or antigen binding fragments thereof) specific for ARSB, small molecule activators specific for ARSB, small molecule activators specific for the receptor of ARSB, and/or stimulatory aptamers that specifically bind ARSB.

In one embodiment, the agonist of SORCS2 is an activator of SORCS2, which may include, e.g., compositions that increase the expression or functional activity of SORCS2. Such activators can target SORCS2 directly, or can target receptors which bind SORCS2 and consequently mediate the function of SORCS2. Exemplary activators of SORCS2 can include, but are not limited to, agonistic antibodies (or antigen binding fragments thereof) specific for SORCS2, small molecule activators specific for SORCS2, small molecule activators specific for the receptor of SORCS2, and/or stimulatory aptamers that specifically bind SORCS2.

In some embodiments, the subject has previously undergone bariatric surgery, wherein the bariatric surgery is gastric bypass, roux-en-Y gastric bypass, biliopancreatic bypass, duodenal switch, gastric banding, gastrectomy, sleeve gastrectomy, fundoplication, or other gastrointestinal surgical procedures. In another embodiment, the subject has reactive hypoglycemia.

In one embodiment, the therapeutic methods described herein are performed in a human In a further embodiment, the methods described herein are not performed on a mouse or other non-human animal.

The antagonists of the glucose modulating molecules, e.g., FGF19, IGFBP1, ADIPOQ, GCG, SHBG, CXCL3, CXCL2, TNFRSF17, AMICA1, TFF3, EFNB3 and LSAMP, and/or the agonists of the glucose modulating molecules, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2, as described herein can be administered by any suitable means, including parenteral administration (e.g., injection, infusion), and may be by subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the antagonist of the glucose modulating molecule, e.g., FGF19, IGFBP1, ADIPOQ, GCG, SHBG, CXCL3, CXCL2, TNFRSF17, AMICA1, TFF3, EFNB3 and LSAMP, and/or the agonist of the glucose modulating molecule, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2, is suitably administered by pulse infusion, particularly with declining doses. The dosing can be given by injections, such as intravenous or subcutaneous injections. The route of administration can be selected according to various factors, such as whether the administration is brief or chronic. Other administration methods are contemplated, including topical, particularly transdermal, transmucosal, rectal, oral or local administration e.g. through a catheter placed close to the desired site. Injection, especially intravenous, is of interest.

In some embodiments, the methods comprises administering a therapeutically effective amount of an antagonist for the glucose modulating molecule, e.g., FGF19, IGFBP1, ADIPOQ, GCG, SHBG, CXCL3, CXCL2, TNFRSF17, AMICA1, TFF3, EFNB3 and LSAMP as described herein to the subject. In some embodiments, the methods comprises administering a therapeutically effective amount of an agonist for the glucose modulating molecule, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2 as described herein to the subject.

In some embodiments, the methods comprises administering a therapeutically effective amount of an FGF19 antagonist as described herein to the subject. The therapeutically effective amount of a therapeutic agent, or combinations thereof, is an amount sufficient to treat disease in a subject. For example, for FGF19 antagonist, a therapeutically effective amount can be an amount that provides an observable therapeutic benefit compared to baseline clinically observable signs and symptoms of hypoglycemia, e.g., by increasing blood glucose levels. The therapeutically effective dosage of antagonists and/or agonists of the glucose modulating molecules as described herein will vary somewhat from subject to subject, and will depend upon factors such as the age, weight, and condition of the subject and the route of delivery. Such dosages can be determined in accordance with procedures known to those skilled in the art.

### II.E. Diagnostic Uses of Glucose Modulating Molecules

Current methods of identifying subjects having or at risk for hypoglycemia rely on a combination of factors such as patient medical history, blood glucose and insulin levels at fasting or after meals. The present invention provides an improved method for determining whether a subject has or is at risk for hypoglycemia, e.g., post-bariatric hypoglycemia (PBH), based, at least in part, on the discovery that the expression levels of certain biomarkers identified herein (see, e.g., biomarkers described in FIGS. 5A and 5B) are either elevated or reduced in patients with post-bariatric hypoglycemia. Thus, the invention may be used to determine whether a human subject has or is at risk of developing post-bariatric hypoglycemia.

The invention identifies certain biomarkers associated with post-bariatric hypoglycemia which may be used to determine whether a subject is at risk for developing such a disorder. Such predictive means benefit the overall health of the subject, as faster responses can be made to determine the appropriate therapy. The methods described herein also decrease the overall cost of the treatment process by more quickly eliminating ineffective therapies.

Generally, in some embodiments, the methods of the invention include determining the levels of glucose modulating molecules as described herein in a sample obtained from a subject who is considering or has undergone bariatric surgery, and comparing the levels of biomarkers in the sample to a suitable control, to determine whether the subject's glucose modulating molecule level is increased, decreased, or the same, relative to the control.

The term “control” refers to an accepted or pre-determined level (e.g., mRNA level or protein level) of the biomarker which is used to determine whether or not the level of a biomarker in a biological sample derived from a test subject is different from the level of the biomarker present in a normal subject, e.g., a subject who does not have hypoglycemia, e.g., a subject who does not have PBH. The skilled person can select an appropriate control for the assay in question. For example, a control may be a biological sample derived from a known subject, e.g., a subject known to be a normal subject, or a subject known to have hypoglycemia. If a control is obtained from a normal subject, a statistically significant difference in the level of a biomarker described herein in a test subject relative to the control is indicative that the subject has hypoglycemia. If a control is obtained from a subject known to have hypoglycemia, levels comparable to such a control are indicative of hypoglycemia, reflective of a difference in the levels present in a sample from a normal subject. In one embodiment, the difference in the level of a biomarker of hypoglycemia, e.g., FGF19, IGFBP1, ADIPOQ, GCG, SHBG, CXCL3, CXCL2, TNFRSF17, AMICA1, TFF3, EFNB3 and/or LSAMP, is an increase relative to the level present in a sample from a normal subject. In one embodiment, the difference in the level of a biomarker of hypoglycemia, e.g., HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and/or SORCS2 is a decrease relative to the level present in a sample from a normal subject.

In one embodiment, a control may also be a reference standard. A reference standard serves as a reference level for comparison, such that test samples can be compared to the reference standard in order to infer the disease status of a subject. A reference standard may be representative of the level of one or more biomarkers in a known subject, e.g., a subject known to be a normal subject, or a subject known to have hypoglycemia. Likewise, a reference standard may be representative of the level of one or more biomarkers in a population of known subjects, e.g., a population of subjects known to be normal subjects, or a population of subjects known to have hypoglycemia. The reference standard may be obtained, for example, by pooling samples from a plurality of individuals and determining the level of a biomarker in the pooled samples, to thereby produce a standard over an averaged population. Such a reference standard represents an average level of a biomarker among a population of individuals. A reference standard may also be obtained, for example, by averaging the level of a biomarker determined to be present in individual samples obtained from a plurality of individuals. Such a standard is also representative of an average level of a biomarker among a population of individuals. A reference standard may also be a collection of values each representing the level of a biomarker in a known subject in a population of individuals. In certain embodiments, test samples may be compared against such a collection of values in order to infer the disease status of a subject. In certain embodiments, the reference standard is an absolute value. In such embodiments, test samples may be compared against the absolute value in order to infer whether a subject has or is at risk for hypoglycemia. In a one embodiment, a comparison between the level of one or more biomarkers in a sample relative to a control is made by executing a software classification algorithm. The skilled person can readily envision additional controls that may be appropriate depending on the assay in question. The aforementioned controls are exemplary, and are not intended to be limiting.

In one embodiment, the invention provides a method for determining whether a subject has or is at risk for post-bariatric hypoglycemia. The methods comprises determining the level of a glucose modulating molecule in a sample obtained from a subject who is considering or has undergone bariatric surgery, and comparing the level of the glucose modulating moleculein the sample to a suitable control, wherein the glucose moculating molecule is FGF19, IGFBP1, ADIPOQ, GCG, SHBG, CXCL3, CXCL2, TNFRSF17, AMICA1, TFF3, EFNB3 or LSAMP, or combinations thereof, where an increase in the level of the glucose modulating molecules described herein in the sample relative to the suitable control is indicative that the subject has or is at risk for post-bariatric hypoglycemia, and wherein no change or a decrease in the level of the glucose modulating moleculeas described herein in the sample relative to the suitable control is indicative that the subject does not have and/or is not at risk for post-bariatric hypoglycemia.

In another embodiment, the invention provides a method for determining whether a subject has or is at risk for post-bariatric hypoglycemia. The methods comprises determining the level of FGF19 in a sample obtained from a subject who is considering or has undergone bariatric surgery, and comparing the level of FGF19 in the sample to a suitable control, where an increase in the level of the biomarkers as described herein in the sample relative to the suitable control is indicative that the subject has or is at risk for post-bariatric hypoglycemia, and wherein no change or a decrease in the level of the biomarkers as described herein in the sample relative to the suitable control is indicative that the subject does not have and/or is not at risk for post-bariatric hypoglycemia.

In yet another embodiment, the invention provides a method for determining whether a subject has or is at risk for post-bariatric hypoglycemia. The methods comprises determining the level of a glucose modulating molecule in a sample obtained from a subject who is considering or has undergone bariatric surgery, and comparing the level of the glucose modulating moleculein the sample to a suitable control, wherein the glucose moculating molecule is HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, SORCS2, and combinations thereof, where a decrease in the level of the glucose modulating moleculeas as described herein in the sample relative to the suitable control is indicative that the subject has or is at risk for post-bariatric hypoglycemia, and wherein no change or an increase in the level of the glucose modulating moleculeas as described herein in the sample relative to the suitable control is indicative that the subject does not have and/or is not at risk for post-bariatric hypoglycemia.

In another embodiment, the invention provides a method of selecting a bariatric surgery for a subject having obesity, comprising comparing the level of one or more glucose modulating molecule(s) selected from FGF19, IGFBP1, ADIPOQ, GCG, SHBG, CXCL3, CXCL2, TNFRSF17, AMICA1, TFF3, EFNB3, LSAMP, and combinations thereof, in a sample obtained from the subject, to a control level of the glucose modulating molecule representative of the level in a comparable sample from a subject who does not have or is not at risk for post-bariatric hypoglycemia (PBH), and selecting a bariatric surgery for the subject if the level of the one or more glucose modulating molecule(s) in the sample obtained from the subject is equivalent to or lower than the control level of the one or more glucose modulating molecules. The bariatric surgery can include, for example, gastric bypass, roux-en-Y gastric bypass, biliopancreatic bypass, duodenal switch, gastric banding, gastrectomy, sleeve gastrectomy, or fundoplication. A treatment other than bariatric surgery can selected for a subject having obesity if the level of the one or more glucose modulating molecule(s) in the sample obtained from the subject is higher than the control level of the one or more glucose modulating molecules.

In another embodiment, the invention provides a method of selecting a bariatric surgery for a subject having obesity, comprising comparing the level of one or more glucose modulating molecule(s) selected from the group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB, SORCS2, and combinations thereof, in a sample obtained from the subject to a control level of the glucose modulating molecule representative of the level in a comparable sample from a subject who does not have or is not at risk for post-bariatric hypoglycemia (PBH), and selecting a bariatric surgery for the subject if the level of the one or more glucose modulating molecule(s) in the sample obtained from the subject is equivalent to or higher than the control level of the one or more glucose modulating molecules. The bariatric surgery can include, for example, gastric bypass, roux-en-Y gastric bypass, biliopancreatic bypass, duodenal switch, gastric banding, gastrectomy, sleeve gastrectomy, or fundoplication. A treatment other than bariatric surgery can be selected for a subject having obesity if the level of the one or more glucose modulating molecule(s) in the sample obtained from the subject is lower than the control level of the one or more glucose modulating molecules.

In one embodiment of the foregoing aspects, the method can further comprise determining the level of the one or more glucose modulating molecule(s) in a sample obtained from the subject.

In some embodiments, the sample is selected from the group consisting of a plasma sample, a serum sample, or a blood sample. In other embodiments, the subject has or is at risk for post-bariatric hypoglycemia is considering or has undergone a bariatric surgery selected from the group consisting of gastric bypass, roux-en-Y gastric bypass, biliopancreatic bypass, duodenal switch, gastric banding, gastrectomy, sleeve gastrectomy, fundoplication, and other gastrointestinal surgical procedures.

In some embodiments, an increased expression level refers to an overall increase of greater than about and/or about any of 5%, 10%, 20%, 25%, 30%, 40%, 50%, 60%, 70%, 80%, 85%, 90%, 95%, 96%, 97%, 98%, 99% or greater, in the level of one or more of the glucose modulating molecule as described in the present invention (e.g., FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2), detected by standard art known methods such as those described herein, as compared to a reference sample, reference cell, reference tissue, control sample, control cell, or control tissue. In certain embodiments, the increased expression level refers to the increase in expression level and/or levels of one or more biomarkers in the sample wherein the increase is at least about any of 1.5×, 1.75×, 2×, 3×, 4×, 5×, 6×, 7×, 8×, 9×, 10×, 25×, 50×, 75×, or 100× the expression level and/or level of the respective biomarker in a reference sample, reference cell, reference tissue, control sample, control cell, or control tissue. In some embodiments, elevated expression levels and/or levels of one or more biomarkers refers to an overall increase of greater than about and/or about any of 1.1-fold, 1.5-fold, 2-fold, 2.5-fold, 3-fold, 4-fold, 5-fold, 10-fold, 12-fold, 15-fold, 17-fold, about 20-fold, 25-fold, and/or 30-fold as compared to a reference sample, reference cell, reference tissue, control sample, control cell, control tissue, or internal control (e.g., housekeeping gene).

In some embodiments of any of the methods, reduced expression level and/or levels refers to an overall reduction of greater than about and/or about any of 5%, 8%, 10%, 20%, 25%, 30%, 35% 40%, 50%, 60%, 64% 70%, 80%, 85%, 90%, 95%, 96%, 97%, 98%, 99% or greater, in the level of one or more biomarkers as described in the present invention (e.g., FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPCS, ARSB, and SORCS2), detected by standard art known methods such as those described herein, as compared to a reference sample, reference cell, reference tissue, control sample, control cell, or control tissue. In certain embodiments, the reduced expression levels and/or levels refers to the decrease in expression level and/or levels of one or more biomarkers in the sample wherein the decrease is at least about any of 1.5×, 1.75×, 2×, 3×, 4×, 5×, 6×, 7×, 8×, 9×, 10×, 25×, 50×, 75×, or 100× the expression level and/or level of the respective biomarker in a reference sample, reference cell, reference tissue, control sample, control cell, or control tissue. In certain embodiments, reduced expression levels and/or levels refers to the decrease in expression level and/or levels of one or more biomarkers in the sample wherein the decrease is at least about and/or about any of 0.9×, 0.8×, 0.7×, 0.6×, 0.5×, 0.4×, 0.3×, 0.2×, 0.1×, 0.05×, or 0.01× the expression level/level of the respective biomarker in a reference sample, reference cell, reference tissue, control sample, control cell, or control tissue.

In some embodiments, an increase in the level of one or more glucose modulating molecules, e.g., FGF19, IGFBP1, ADIPOQ, GCG, SHBG, CXCL3, CXCL2, TNFRSF17, AMICA1, TFF3, EFNB3 and LSAMP, in the sample relative to the suitable control identifies the subject as a candidate for treatment with an antagonist of the glucose modulating molecule as described herein.

In some embodiments, an increase in the level of FGF19 in the sample relative to the suitable control identifies the subject as a candidate for treatment with an FGF19 antagonist as described herein.

In another embodiment, the methods comprises administering a therapeutically effective amount of an antagonist of the glucose modulating molecule as described herein to the subject. In a further embodiment, the methods comprises administering a therapeutically effective amount of an FGF19 antagonist as described herein to the subject. The therapeutically effective amount of a therapeutic agent, or combinations thereof, is an amount sufficient to treat disease in a subject. For example, for FGF19 antagonist, a therapeutically effective amount can be an amount that provides an observable therapeutic benefit compared to baseline clinically observable signs and symptoms of hypoglycemia, e.g., by increasing blood glucose levels.

In some embodiments, a decrease in the level of the glucose modulating molecule, e.g., HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2, in the sample relative to the suitable control identifies the subject as a candidate for treatment with an agonist of the glucose modulating molecule as described herein.

In another embodiment, the methods comprises administering a therapeutically effective amount of an agonist of the glucose modulating molecule, e.g., HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2, to the subject. The therapeutically effective amount of a therapeutic agent, or combinations thereof, is an amount sufficient to treat disease in a subject. For example, for an agonist of the glucose modulating molecule as described here, a therapeutically effective amount can be an amount that provides an observable therapeutic benefit compared to baseline clinically observable signs and symptoms of hypoglycemia, e.g., by increasing blood glucose levels.

Expression of the glucose modulating molecules as described herein (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2) can be detected at both the RNA level and the protein level using methods known to those skilled in the art. The methods of the invention may be performed using protein-based assays to determine the level of the given marker. Examples of protein-based assays include immunohistochemical and/or Western analysis, quantitative blood based assays, e.g., serum ELISA, and quantitative urine based assays, e.g., urine ELISA. In one embodiment, an immunoassay is performed to provide a quantitative assessment of the given marker.

Proteins from samples can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

The amount of marker may be determined by detecting or quantifying the corresponding expressed polypeptide. The polypeptide can be detected and quantified by any of a number of means well known to those of skill in the art. These may include analytic biochemical methods such as electrophoresis, capillary electrophoresis, high performance liquid chromatography (HPLC), mass spectrometry, thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, and Western blotting.

The methods of the invention may be performed using protein-based assays to determine the level of the given biomarker. Examples of protein-based assays include immunohistochemical and/or Western analysis, quantitative blood based assays, e.g., serum ELISA, and quantitative urine based assays, e.g., urine ELISA. In one embodiment, an immunoassay is performed to provide a quantitative assessment of the given biomarker.

Proteins from patient samples can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

The amount of the glucose modulating molecules as described herein (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2) may be determined by detecting or quantifying the corresponding expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, and Western blotting.

In one embodiment the level of the glucose modulating molecules as described herein (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2) may be determined using an immunoassay. The use of antibodies directed to biomarkers described herein can be used to screen human biological samples, e.g., fluids, for the levels of the specific glucose modulating molecules as described herein (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2). By way of illustration, human fluids, such as blood serum or urine, can be taken from a patient and assayed for a specific epitope, either as released antigen or membrane-bound on cells in the sample fluid, using anti-biomarker antibodies in standard RIAs or ELISAs, for example, known in the art. In immunoassays, the agent for detecting the polypeptide and polypeptides encoding the glucose modulating molecules (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2) may be an antibody capable of binding to the protein of the glucose modulating molecules as described herein. Antibodies can be polyclonal, or more preferably, monoclonal. An intact antibody, or a fragment thereof (e.g., Fab or F(ab′)₂) can be used.

Competitive binding assays may be used to determine the level of the protein corresponding to the glucose modulating molecules (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2). One example of a competitive binding assay is an enzyme-linked immunosorbent sandwich assay (ELISA). ELISA can be used to detect the presence of the glucose modulating molecules (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2) in a sample. ELISA is a sensitive immunoassay that uses an enzyme linked to an antibody or antigen as a marker for the detection of a specific protein, especially an antigen or antibody. ELISA is an assay wherein bound antigen or antibody is detected by a linked enzyme that generally converts a colorless substrate into a colored product, or a product which can be detected. One of the most common types of ELISA is “sandwich ELISA.” In one embodiment, the level of the glucose modulating molecule, e.g., FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and/or SORCS2 is determined using an ELISA assay. In addition, a skilled artisan can readily adapt known protein/antibody detection methods for use in determining the amount of a marker of the present invention. Antibodies used in immunoassays known in the art and described herein to determine levels of biomarkers, may be labeled with a detectable label. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a one embodiment, the antibody is labeled, e.g. a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with the glucose modulating molecules (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2).

In one embodiment of the invention, proteomic methods, e.g., mass spectrometry, are used for detecting and quantitating the glucose modulating molecules (e.g. FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPC5, ARSB, and SORCS2). For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate glucose modulating molecules. Mass spectrometric methods are described in, for example, U.S. Pat. Nos. 5,622,824, 5,605,798 and 5,547,835, the entire contents of each of which are incorporated herein by reference.

In one embodiment, the level of the glucose modulating molecules as described herein can be measured at the RNA level using methods known to those skilled in the art, e.g. Northern analysis. Gene expression of the biomarker can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting and In Situ hybridization. Gene expression can also be detected by microarray analysis as described below.

For Northern blotting, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

Nuclease Protection Assays (including both ribonuclease protection assays and S1 nuclease assays) provide an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. NPAs allow the simultaneous detection of several RNA species.

In situ hybridization (ISH) is a powerful and versatile tool for the localization of specific mRNAs in cells or tissues. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents. This latter method of detection is the basis for Fluorescent In Situ Hybridisation (FISH).

Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection. Relative quantitative RT-PCR involves amplifying an internal control simultaneously with the gene of interest. The internal control is used to normalize the samples. Once normalized, direct comparisons of relative abundance of a specific mRNA can be made across the samples. Commonly used internal controls include, for example, GAPDH, HPRT, actin and cyclophilin.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification (TAS) methods have been described in the literature, for example, general reviews of these methods in Landegren, U. et al., Science 242:229-237 (1988) and Lewis, R., Genetic Engineering News 10:1, 54-55 (1990). PCR is a nucleic acid amplification method common in the art and described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the Q.beta. Replicase technique, RNA replicase for the bacteriophage Q.beta., which replicates single-stranded RNA, is used to amplify the target DNA, as described by Lizardi et al., 1988, Bio/Technology 6:1197. Quantitative PCR (Q-PCR) is a technique which allows relative amounts of transcripts within a sample to be determined.

### II.F. Kits

The invention also provides kits for the treatment and/or diagnosis of the disorders described above. Such kits include means for determining the level of expression of a glucose modulating molecules and instructions for use of the kit. For example, in particular embodiments, a kit of the invention includes means for determining the level of FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPCS, ARSB, and SORCS2 or combinations thereof. In one embodiment, a kit of the invention includes means for determining the level of one or more of the following biomarkers: FGF19, IGFBP1, ADIPOQ, GCG, SHBG, CXCL3, CXCL2, TNFRSF17, AMICA1, TFF3, EFNB3, LSAMP, HGFAC, BMPR2, GDF11, IGFBP7, IGFBP6, APOE, PLA2G7, CDK2, CCNA1, MAPKAPK3, KLK3, PLAT, CCL3L1, CCL27, CD97, AFM, RTN4R, GNLY, PFD5, MB, GPCS, ARSB, and SORCS2.

Kits of the invention can optionally contain additional components useful for performing the methods of the invention. For example, the kits may include means for obtaining and/or processing a biological sample from a subject. Means for isolating a biological sample from a subject can comprise one or more reagents that can be used to obtain a fluid or tissue from a subject, such as reagents that can be used to obtain or collect a cell or tissue sample from a subject. Means for processing a biological sample from a subject can include one or more reagents that can be used to transform a biological sample such that the level of one or more biomarkers in the sample can be determined. Such reagents can include, for example, reagents for isolating DNA from a biological sample, reagents for isolating RNA from a biological sample, and/or reagents for isolating protein from a biological sample.

Means for determining the level of a biomarker can include, for example, reagents for detecting the presence or level of a gene, an RNA transcribed from a gene, or a protein encoded by a gene. Such reagents include, but are not limited to, probes or primers that specifically hybridize to a nucleic acid sequence of a gene, and/or antibodies or antigen-binding portions thereof that specifically bind to a protein encoded by a gene. Buffers or other reagents necessary for evaluating expression of a biomarker (e.g., at the DNA, RNA, or protein level) may also be included in the kits of the invention. Instructions can include steps for performing an assay for evaluating the level of expression of one or more (e.g., two or more, three or more, four or more, five or more, etc.) biomarkers in a biological sample. In preferred embodiments, the kits are designed for use with a human subject.

The invention is illustrated by the following example, which is not intended to be limiting in any way.

### EXAMPLE

### Identification of Novel Mediators of Hypoglycemia

In order to identify novel mediators of hypoglycemia following gastric bypass, detailed metabolic analysis of plasma samples collected from patients with post-bariatric hypoglycemia (PBH) was performed. Plasma samples from aymptomatic individuals who have had gastric bypass, but did not develop hypoglycemia, were also analyzed and used as a control. Patients were admitted to the research center after an overnight fast, and blood samples were collected. Patients were provided a mixed liquid meal containing carbohydrates, protein and fat, and blood sampling was performed at intervals up to 120 minutes later.

Plasma samples collected during this mixed meal testing were analyzed using the Somalogic platform for sensitive proteomic analysis (Rohloff, J. C. et al. Mol Ther Nucleic Acids 3, e201, 2014). This approach utilized a highly multiplexed, sensitive platform to measure 1129 analytes simultaneously, using modified aptamers (which are single-stranded DNA which bind specific plasma proteins in their native configuration). Bound proteins were quantified using microarray-based detection of nucleotide portion of the aptamer.

Preliminary analysis of the plasma proteome using the Somalogic platform identified several proteins being modified in patients with PBH which could contribute to insulin-independent metabolic changes and serve as novel therapeutic targets for modulating glucose, particularly for the treatment of hypoglycemia. FIG. 5A describes proteins that were upregulated in PBH patients, including proteins associated with hormone signaling and metabolic regulation, (i.e., FGF19, IGFBP1, ADIPOQ, GCG, and SHBG), proteins associated with inflammation (i.e., CXCL3, CXCL2, TNFRSF17, and AMICA1), and proteins associated with developmental regulation (i.e., TFF3, EFNB3, and LSAMP). FIG. 5B describes proteins that were downregulated in PBH patients, including proteins associated with hormone signaling and metabolic regulation (i.e., HGFAC, BMPR2, GDF11, IGFBP7, and IGFBP6), proteins associated with lipid metabolism (i.e., APOE and PLA2G7), proteins associated with cell cycle regulation (CDK2, CCNA2, and MAPKAPK3), proteases (i.e., KLK3 and PLAT), cytokines (i.e., CCL3L1 and CCL27), and CD97, AFM, RTN4R, GNLY, PFD5, MB, GPC5, ARSB, and SORCS2. In FIGS. 5A and 5B, proteins differentially regulated at multiple timepoints are indicated by underlining In FIGS. 5A and 5B, italics indicate those proteins that are differentially regulated in asymptomatic patients with history of gastric bypass as compared with nonsurgical controls. As such, the italicized proteins may contribute to not only improvements in glucose metabolism following bariatric surgery, but also the “extreme” lowering of plasma glucose in patients with PBH. Notable in both up-regulated and down-regulated proteins described in FIGS. 5A and 5B are the functional overrepresentation of proteins regulating hormonal signaling and systemic metabolism. Upregulated proteins include FGF19, IGFBP1 (IGF1 binding protein), ADIPOQ (adiponectin, an abundant plasma protein for which upregulation improves systemic metabolism and insulin resistance), glucagon (pancreatic islet regulator of glucose homeostasis), and SHBG (a known marker of and genetic locus linked to systemic insulin sensitivity). Elevations in both adiponectin and glucagon were confirmed in prior studies in this population, providing validation of assay results. Downregulated proteins include regulators of hepatocyte growth factor, BMP/TGF-related signaling (BMPR2 and GDF11), and additional IGFBP—all of which can modulate systemic metabolism and are thus identified as candidate molecules contributing to PBH.

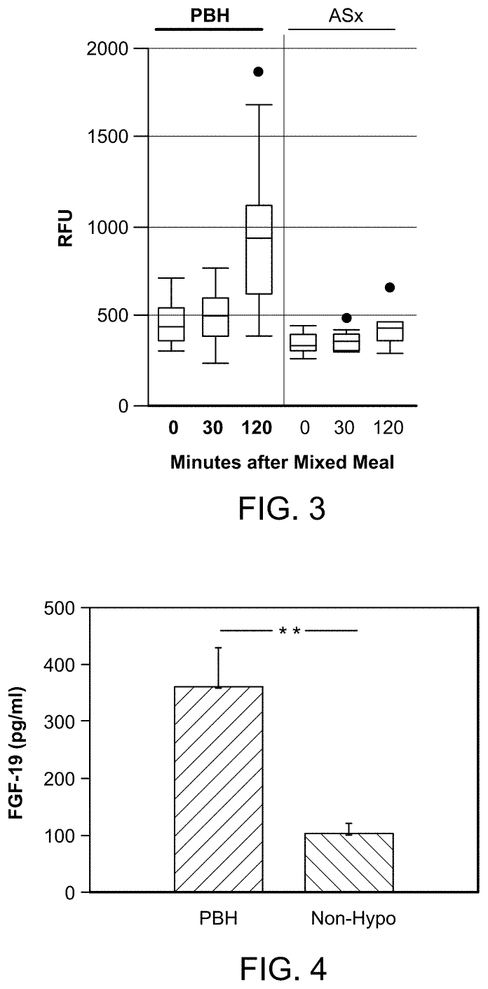

One protein identified in this analysis as a contributor to the pathogenesis of PBH is FGF19. FGF19 levels were markedly increased in PBH vs. asymptomatic post-bypass patients, most dramatically at 120 minutes after mixed meal ingestion (2.1 fold, p<0.01), as described in FIG. 3. These data were further confirmed by an ELISA analysis in a subset of patients. As shown in FIG. 4, there was a 3.5 fold increase in the level of FGF19 protein in patients with hypoglycemia as compared with those without hypoglycemia (p<0.0001), suggesting that FGF19 can serve as a novel mediator of for hypoglycemia in PBH.

Increases in FGF19 were of particular importance and interest as FGF19 is secreted by enterocytes in response to bile acid-stimulated activation of FXR. Several lines of converging evidence implicate the bile acid-FXR-FGF axis in post-bypass metabolic responses: (a) postprandial plasma bile acids are increased by 2.5 fold in post-bypass patients, correlating with postprandial GLP1 (r=0.58,p<0.01) and inversely with glucose (r=−0.59, p<0.01) (Patti, M. E. et al. Obesity (Silver. Spring), 2009); (b) specific bile acid species are altered in Type 2 Diabetes (T2D) (Wewalka, M. et al, J. Clin. Endocrinol. Metab 99, 1442-1451, 2014); (c) FXR is required for metabolic effects of bariatric surgery in rodents (Ryan, K. K. et al. Nature 509, 183-188, 2014); (d) Mice lacking FGF15 (rodent homolog of FGF19) are glucose intolerant (Kir, S. et al. Science 331, 1621-1624, 2011); (e) FGF19 is stimulated by nutrient load, particularly carbohydrates (Morton, G. J. et al, Clin Endocrinol Metab 99, E241-E245, 2014) and by bile acids, via FXR-dependent mechanism (Holt, J. A. et al. Genes Dev 17, 1581-1591, 2003); (f) FGF19 levels are reduced in T2D (Roesch, S. L. et al. PLoS One 10, e0116928, 2015); (g) FGF19 levels can contribute to insulin-dependent glucose disposal (Morton, G. J. et al. J Clin Invest 123, 4799-4808, 2013), as recently reported in patients with hypoglycemia (Patti, M. E., Li, P., & Goldfine, A. B. Obesity (Silver. Spring), 2015). Thus, increased postprandial FGF19 levels could contribute to postprandial hypoglycemia.

### Equivalents

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method of increasing the blood glucose level of a subject in need thereof, comprising administering an antagonist of a glucose modulating molecule to the subject, such that the blood glucose level of the subject is increased, wherein the glucose modulating molecule is selected from a group consisting of FGF19, IGFBP1, ADIPOQ, GCG, SHBG, CXCL3, CXCL2, TNFRSF17, AMICA1, TFF3, EFNB3 and LSAMP.

2. A method of treating or preventing hypoglycemia in a subject in need thereof, comprising administering an antagonist of a glucose modulating molecule to the subject, such that hypoglycemia is treated or prevented, wherein the glucose modulating molecule is selected from a group consisting of FGF19, IGFBP1, ADIPOQ, GCG, SHBG, CXCL3, CXCL2, TNFRSF17, AMICA1, TFF3, EFNB3 and LSAMP.

3. The method of claim 1, wherein the subject has undergone bariatric surgery.

4. The method of claim 3, wherein the bariatric surgery is selected from the group consisting of gastric bypass, roux-en-Y gastric bypass, biliopancreatic bypass, duodenal switch, gastric banding, gastrectomy, sleeve gastrectomy, fundoplication, and other gastrointestinal surgical procedures.

5. The method of claim 1, wherein the subject has reactive hypoglycemia.

6. The method of claim 1, wherein the antagonist of the glucose modulating molecule is selected from the group consisting of
an antibody, or an antigen binding fragment thereof, which specifically binds the glucose modulating molecule,
a soluble form of a receptor specific for the glucose modulating molecule,
a small molecule inhibitor specific for the glucose modulating molecule,
an antisense oligonucleotide specific for the glucose modulating molecule,
and an inhibitory aptamer that specifically binds the glucose modulating molecule.

7. The method of claim 1, wherein the glucose modulating molecule is FGF19.

8. The method of claim 1, wherein the glucose modulating molecule is FGF19 and the antagonist of FGF19 is an inhibitor of an FGF19 receptor.

9. The method of claim 8, wherein the FGF19 receptor is FGFR4 or Klotho.

10. The method of claim 8, wherein the inhibitor of the FGF19 receptor is selected from the group consisting of an anti-FGFR4 antibody, or an antigen binding fragment thereof, a small molecule inhibitor specific for FGFR4, an antisense oligonucleotide specific for FGFR4, an aptamer that specifically binds FGFR4, an anti-Klotho antibody, or an antigen binding fragment thereof, a small molecule inhibitor specific for Klotho, an antisense oligonucleotide specific for Klotho, and an aptamer that specifically binds Klotho.

11. A method of increasing the blood glucose level of a subject in need thereof, comprising administering an agonist of a glucose modulating molecule to the subject, wherein the glucose modulating molecule is selected from a group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPCS, ARSB and SORCS2, such that the blood glucose level of the subject is increased.

12. A method of treating or preventing hypoglycemia in a subject in need thereof, comprising administering an agonist of a glucose modulating molecule to the subject, such that hypoglycemia is treated or prevented, wherein the glucose modulating molecule is selected from a group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPCS, ARSB and SORCS2.

13. The method of claim 11, wherein the subject has undergone bariatric surgery.

14. The method of claim 13, wherein the bariatric surgery is selected from the group consisting of gastric bypass, roux-en-Y gastric bypass, gastrectomy, sleeve gastrectomy, and fundoplication.

15. The method of claim 13, wherein the subject has reactive hypoglycemia.

16. The method of claim 11, wherein the agonist of the glucose modulating molecule is selected from the group consisting of
an agonist antibody, or an antigen binding fragment thereof, which specifically binds the glucose modulating molecule,
a small molecule specific for the glucose modulating molecule,
and a stimulatory aptamer that specifically binds a glucose modulating molecule.

17. The method of claim 11, wherein the agonist of the glucose modulating molecule is a protein selected from the group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPCS, ARSB and SORCS2, or a nucleic acid encoding a protein selected from the group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2.

18. A method of determining whether a subject has or is at risk for having post-bariatric hypoglycemia (PBH), comprising:
determining the level of one or more glucose modulating molecule(s) in a sample obtained from the subject, wherein the glucose modulating molecule is selected from a group consisting of FGF19, IGFBP1, ADIPOQ, GCG, SHBG, CXCL3, CXCL2, TNFRSF17, AMICA1, TFF3, EFNB3, LSAMP, and combinations thereof; and
comparing the level of the glucose modulating molecule(s) in the sample to a control level of the glucose modulating molecule from a subject who does not have or is not at risk for having PBH;
wherein an increase in the level of the glucose modulating molecule(s) in the sample relative to the control level is indicative that the subject has or is at risk for post-bariatric hypoglycemia; and
wherein no change or a decrease in the level of the glucose modulating molecule in the sample relative to the control is indicative that the subject does not have or is not at risk for post-bariatric hypoglycemia.

19. (canceled)

20. The method of claim 18, wherein the sample is a blood sample.

21. (canceled)

22. The method of claim 18, further comprising administering a therapeutically effective amount of
an antagonist of a glucose modulating molecule to the subject, wherein the glucose modulating molecule is selected from a group consisting of FGF19, IGFBP1, ADIPOQ, GCG, SHBG, CXCL3, CXCL2, TNFRSF17, AMICA1, TFF3, EFNB3 and LSAMP; and/or
an agonist of a glucose modulating molecule to the subject, wherein the glucose modulating molecule is selected from a group consisting of HGFAC, BMPR2, GDF11, IGFBP7, IGFBP6, APOE, PLA2G7, CDK2, CCNA2, MAPKAPK3, KLK3, PLAT, CCL3L1, CCL27, CD97, AFM, RTN4R, GNLY, PFD5, MB, GPC5, ARSB and SORCS2.

23.-29. (canceled)


SEQUENCE LISTING (82 sequences)

SEQ ID NO: 1 - DNA, 2157 bp, Homo sapiens
  misc_feature (1)..(2157): Homo sapiens fibroblast growth factor 19 (FGF19), mRNA
gctcccagccaagaacctcggggccgctgcgcggtggggaggagttccccgaaacccggccgctaagcgaggcctcctcctcccgcagatccgaacggcctgggcggggtcaccccggctgggacaagaagccgccgcctgcctgcccgggcccggggagggggctggggctggggccggaggcggggtgtgagtgggtgtgtgcggggggcggaggcttgatgcaatcccgataagaaatgctcgggtgtcttgggcacctacccgtggggcccgtaaggcgctactatataaggctgccggcccggagccgccgcgccgtcagagcaggagcgctgcgtccaggatctagggccacgaccatcccaacccggcactcacagccccgcagcgcatcccggtcgccgcccagcctcccgcacccccatcgccggagctgcgccgagagccccagggaggtgccatgcggagcgggtgtgtggtggtccacgtatggatcctggccggcctctggctggccgtggccgggcgccccctcgccttctcggacgcggggccccacgtgcactacggctggggcgaccccatccgcctgcggcacctgtacacctccggcccccacgggctctccagctgcttcctgcgcatccgtgccgacggcgtcgtggactgcgcgcggggccagagcgcgcacagtttgctggagatcaaggcagtcgctctgcggaccgtggccatcaagggcgtgcacagcgtgcggtacctctgcatgggcgccgacggcaagatgcaggggctgcttcagtactcggaggaagactgtgctttcgaggaggagatccgcccagatggctacaatgtgtaccgatccgagaagcaccgcctcccggtctccctgagcagtgccaaacagcggcagctgtacaagaacagaggctttcttccactctctcatttcctgcccatgctgcccatggtcccagaggagcctgaggacctcaggggccacttggaatctgacatgttctcttcgcccctggagaccgacagcatggacccatttgggcttgtcaccggactggaggccgtgaggagtcccagctttgagaagtaactgagaccatgcccgggcctcttcactgctgccaggggctgtggtacctgcagcgtgggggacgtgcttctacaagaacagtcctgagtccacgttctgtttagctttaggaagaaacatctagaagttgtacatattcagagttttccattggcagtgccagtttctagccaatagacttgtctgatcataacattgtaagcctgtagcttgcccagctgctgcctgggcccccattctgctccctcgaggttgctggacaagctgctgcactgtctcagttctgcttgaatacctccatcgatggggaactcacttcctttggaaaaattcttatgtcaagctgaaattctctaattttttctcatcacttccccaggagcagccagaagacaggcagtagttttaatttcaggaacaggtgatccactctgtaaaacagcaggtaaatttcactcaaccccatgtgggaattgatctatatctctacttccagggaccatttgcccttcccaaatccctccaggccagaactgactggagcaggcatggcccaccaggcttcaggagtaggggaagcctggagccccactccagccctgggacaacttgagaattccccctgaggccagttctgtcatggatgctgtcctgagaataacttgctgtcccggtgtcacctgcttccatctcccagcccaccagccctctgcccacctcacatgcctccccatggattggggcctcccaggccccccaccttatgtcaacctgcacttcttgttcaaaaatcaggaaaagaaaagatttgaagaccccaagtcttgtcaataacttgctgtgtggaagcagcgggggaagacctagaaccctttccccagcacttggttttccaacatgatatttatgagtaatttattttgatatgtacatctcttattttcttacattatttatgcccccaaattatatttatgtatgtaagtgaggtttgttttgtatattaaaatggagtttgtttgtaaaaaaaaaaaaaaaaaaaa

SEQ ID NO: 2 - protein, 216 aa, Homo sapiens
  misc_feature (1)..(216): fibroblast growth factor 19 precursor
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 3 - DNA, 3115 bp, Homo sapiens
  misc_feature (1)..(3115): Homo sapiens fibroblast growth factor receptor 4 (FGFR4), transcript variant 1, mRNA
gacaggaggtgggccgctcgcggccacgccgccgtcgcgggtacattcctcgctcccggccgaggagcgctcgggctgtctgcggaccctgccgcgtgcaggggtcgcggccggctggagctgggagtgaggcggcggaggagccaggtgaggaggagccaggaaggcagttggtgggaagtccagcttgggtccctgagagctgtgagaaggagatgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcgtctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacccctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccgggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatgagcaaggctcaaggctgtgcaggcacataggctggtggccttgggccttggggctcagccacagcctgacacagtgctcgaccttgatagcatggggcccctggcccagagttgctgtgccgtgtccaagggccgtgcccttgcccttggagctgccgtgcctgtgtcctgatggcccaaatgtcagggttctgctcggcttcttggaccttggcgcttagtccccatcccgggtttggctgagcctggctggagagctgctatgctaaacctcctgcctcccaataccagcaggaggttctgggcctctgaaccccctttccccacacctccccctgctgctgctgccccagcgtcttgacgggagcattggcccctgagcccagagaagctggaagcctgccgaaaacaggagcaaatggcgttttataaattatttttttgaaataaagctctgtgtgcctgggaaaaaaaaaaaaaaaaaaaaaaaaa

SEQ ID NO: 4 - protein, 802 aa, Homo sapiens
  misc_feature (1)..(802): fibroblast growth factor receptor 4 isoform 1 precursor
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 5 - DNA, 6079 bp, Homo sapiens
  misc_feature (1)..(6079): Homo sapiens klotho beta (KLB), mRNA
atcctcagtctcccagttcaagctaatcattgacagagctttacaatcacaagcttttactgaagctttgataagacagtccagcagttggtggcaaatgaagccaggctgtgcggcaggatctccagggaatgaatggattttcttcagcactgatgaaataaccacacgctataggaatacaatgtccaacgggggattgcaaagatctgtcatcctgtcagcacttattctgctacgagctgttactggattctctggagatggaagagctatatggtctaaaaatcctaattttactccggtaaatgaaagtcagctgtttctctatgacactttccctaaaaactttttctggggtattgggactggagcattgcaagtggaagggagttggaagaaggatggaaaaggaccttctatatgggatcatttcatccacacacaccttaaaaatgtcagcagcacgaatggttccagtgacagttatatttttctggaaaaagacttatcagccctggattttataggagtttctttttatcaattttcaatttcctggccaaggcttttccccgatggaatagtaacagttgccaacgcaaaaggtctgcagtactacagtactcttctggacgctctagtgcttagaaacattgaacctatagttactttataccactgggatttgcctttggcactacaagaaaaatatggggggtggaaaaatgataccataatagatatcttcaatgactatgccacatactgtttccagatgtttggggaccgtgtcaaatattggattacaattcacaacccatatctagtggcttggcatgggtatgggacaggtatgcatgcccctggagagaagggaaatttagcagctgtctacactgtgggacacaacttgatcaaggctcactcgaaagtttggcataactacaacacacatttccgcccacatcagaagggttggttatcgatcacgttgggatctcattggatcgagccaaaccggtcggaaaacacgatggatatattcaaatgtcaacaatccatggtttctgtgcttggatggtttgccaaccctatccatggggatggcgactatccagaggggatgagaaagaagttgttctccgttctacccattttctctgaagcagagaagcatgagatgagaggcacagctgatttctttgccttttcttttggacccaacaacttcaagcccctaaacaccatggctaaaatgggacaaaatgtttcacttaatttaagagaagcgctgaactggattaaactggaatacaacaaccctcgaatcttgattgctgagaatggctggttcacagacagtcgtgtgaaaacagaagacaccacggccatctacatgatgaagaatttcctcagccaggtgcttcaagcaataaggttagatgaaatacgagtgtttggttatactgcctggtctctcctggatggctttgaatggcaggatgcttacaccatccgccgaggattattttatgtggattttaacagtaaacagaaagagcggaaacctaagtcttcagcacactactacaaacagatcatacgagaaaatggtttttctttaaaagagtccacgccagatgtgcagggccagtttccctgtgacttctcctggggtgtcactgaatctgttcttaagcccgagtctgtggcttcgtccccacagttcagcgatcctcatctgtacgtgtggaacgccactggcaacagactgttgcaccgagtggaaggggtgaggctgaaaacacgacccgctcaatgcacagattttgtaaacatcaaaaaacaacttgagatgttggcaagaatgaaagtcacccactaccggtttgctctggattgggcctcggtccttcccactggcaacctgtccgcggtgaaccgacaggccctgaggtactacaggtgcgtggtcagtgaggggctgaagcttggcatctccgcgatggtcaccctgtattatccgacccacgcccacctaggcctccccgagcctctgttgcatgccgacgggtggctgaacccatcgacggccgaggccttccaggcctacgctgggctgtgcttccaggagctgggggacctggtgaagctctggatcaccatcaacgagcctaaccggctaagtgacatctacaaccgctctggcaacgacacctacggggcggcgcacaacctgctggtggcccacgccctggcctggcgcctctacgaccggcagttcaggccctcacagcgcggggccgtgtcgctgtcgctgcacgcggactgggcggaacccgccaacccctatgctgactcgcactggagggcggccgagcgcttcctgcagttcgagatcgcctggttcgccgagccgctcttcaagaccggggactaccccgcggccatgagggaatacattgcctccaagcaccgacgggggctttccagctcggccctgccgcgcctcaccgaggccgaaaggaggctgctcaagggcacggtcgacttctgcgcgctcaaccacttcaccactaggttcgtgatgcacgagcagctggccggcagccgctacgactcggacagggacatccagtttctgcaggacatcacccgcctgagctcccccacgcgcctggctgtgattccctggggggtgcgcaagctgctgcggtgggtccggaggaactacggcgacatggacatttacatcaccgccagtggcatcgacgaccaggctctggaggatgaccggctccggaagtactacctagggaagtaccttcaggaggtgctgaaagcatacctgattgataaagtcagaatcaaaggctattatgcattcaaactggctgaagagaaatctaaacccagatttggattcttcacatctgattttaaagctaaatcctcaatacaattttacaacaaagtgatcagcagcaggggcttcccttttgagaacagtagttctagatgcagtcagacccaagaaaatacagagtgcactgtctgcttattccttgtgcagaagaaaccactgatattcctgggttgttgcttcttctccaccctggttctactcttatcaattgccatttttcaaaggcagaagagaagaaagttttggaaagcaaaaaacttacaacacataccattaaagaaaggcaagagagttgttagctaaactgatctgtctgcatgatagacagtttaaaaattcatcccagttccatatgctggtaacttacaggagatatacctgtattatagaaagacaatctgagatacagctgtaaccaaggtgatgacaattgtctctgctgtgtggttcaaagaacattcccttaggtgttgacatcagtgaactcagttcttggatgtaaacataaaggcttcatcctgacagtaagctatgaggattacatgctacattgcttcttaaagtttcatcaactgtattccatcattctgctttagctttcatctctaccaatagctacttgtggtacaataaattatttttaagaagtaaaactctggggctggacgctgtggctcacacctgtaatctcagcactttgggaggccgaggcggggagatcacctgaggtgaggagttcgagaccagcctggccaacatggtgaaaccatgtctctactaaaaatacaaaaaattagccaggcgtggtgacagtggcacctgtaatcccagctacttgggaggctgaggcagaagtttgaacccaggaaacaggttacagtaggccaaaattgcgccactgcactccagcctaggcgacaacagcaagactgtgtccaaaaaaaaaaaaaaaagcaaaagcaaaactttgttttgttagactctacagcagagatttaacacccttctttaaactgggtagtcagtgatagataatatatattctgtcacttctaataaggtgccttctcctttaggtcagggtggttctaaaatggaaagaaaacacaatagggtaagtagtgcttgtctaagccagttacaacacagactcttaaagaggatcaagcccttcatttttctaacaacaaaaaatcacctatagaatatctaatttgtgatcttttactagatctgattttttaaaataatgtaatttccggccaggcacggtggcaccgcctgtaatcccagcactttgggaggccaaggcaggtggatcacctgaggttaggagttcgagactagcctggccaacatggcaaaaccccatctctactaaaaatacaaaagttagccgggcatggtggtgggcacctgtaatcccagctactcaggaggccgaggcaggagaatcgcttgaacccgagaggcagaggttgcaatgagccaagatcgtgccattgcactccagcctgggggacagggcaagactgtctctcaaaataaaaaaaaataataaaaataaaaataaaagtaatttccaaaacctcatctcatggaaagatcacaggatgaaggaaagctagactcaactctgtgaatagaagttgctatactgtaagtaaagcaacaattcagaatactgaatgagtttaaattgttttatatagcacccttttgggctagggttaattactagatctgacttggataatttgacactttgggaaatgaactctgttcttgagacttgttcagtgtattttaaacatctgaggaagaaaacttaaatatgcacctatttatacctattctttctttaggtcaacatttaacacccactgcatacattaatttgtccttgtctgctcactccagcaatttagaccttaacagtcacaagagacgttcttctgttacaaagccttagtaaattaaggcagttttgattatattctaggtccacctatgtctgaagctaaattcagtatctaactgctaatgaacaagtttccaaaatactgtaaaaatacaattagtcaatttgagtaaatgcaaatatgatgagaaatcaatttgctatttggcctggcaaatgggaacagtaaaattctgctttactcttctctagtctccttgccccagctgcacccactaccccaaagttggcagttttgaggtatgattttcaaggaatttttttagtattaacatctccctctgagaactatgtacctaaggtcacgcatacaactagtcaattctgtttttattactctaactatgtagaaacagtaagtcacttaaaacaatcacttggctgggttttttcccctttgtgccacattgattcaccctgacccaagaactccagggaaaattctttaatgtcaactgggcaactcattaacctctctttaacatcaagggcttgggaaaaaaaaaaaaaaggttagccacaggaataacaaaaacctggaatttatctttcaggttttgctttctctttctcactttgtttaaagtatctcgtactcacagttcacaaattaaccttcactgtctctttcacattaagagcttatgcttaaagcatgccccccttttctaacttgctggtttaccataaactcccctaagtaataaaattcctaacccagtactgagagtcctccttctctgccacttgggcattattttactagtttttaagccatcatcgcacaagaatccaaaaacccttaaattttttaaccactggcaaatatgtacagcaaattaggttaagcatttaatctggctcatgctctatcatactaaatattcaggtttatcataaactccttaaaaaccatcaaaggtcaaccagaaactgataactcttgaaaggagcaaacaggtaagatctttggagtttaagcttttctgagatgtgttgtgaaaaatctaacgtgtttatcgtatattcaatgtaacaacctggagaatcacaactatatttaaagagcctctggaaaatgaggccagtacagtgtgactacatgtttaattttcaatgtaatttattccaaataaactggttcatgctgaccacttgtattcaactaa

SEQ ID NO: 6 - protein, 1044 aa, Homo sapiens
  misc_feature (1)..(1044): beta-klotho
MKPGCAAGSPGNEWIFFSTDEITTRYRNTMSNGGLQRSVILSALILLRAVTGFSGCGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGCGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 7 - DNA, 1660 bp, Homo sapiens
  misc_feature (1)..(1660): Homo sapiens insulin-like growth factor binding protein 1 (IGFBP1), mRNA
ggtgcactagcaaaacaaacttattttgaacactcagctcctagcgtgcggcgctgccaatcattaacctcctggtgcaagtggcgcggcctgtgccctttataaggtgcgcgctgtgtccagcgagcatcggccaccgccatcccatccagcgagcatctgccgccgcgccgccgccaccctcccagagagcactggccaccgctccaccatcacttgcccagagtttgggccaccgcccgccgccaccagcccagagagcatcggcccctgtctgctgctcgcgcctggagatgtcagaggtccccgttgctcgcgtctggctggtactgctcctgctgactgtccaggtcggcgtgacagccggcgctccgtggcagtgcgcgccctgctccgccgagaagctcgcgctctgcccgccggtgtccgcctcgtgctcggaggtcacccggtccgccggctgcggctgttgcccgatgtgcgccctgcctctgggcgccgcgtgcggcgtggcgactgcacgctgcgcccggggactcagttgccgcgcgctgccgggggagcagcaacctctgcacgccctcacccgcggccaaggcgcctgcgtgcaggagtctgacgcctccgctccccatgctgcagaggcagggagccctgaaagcccagagagcacggagataactgaggaggagctcctggataatttccatctgatggccccttctgaagaggatcattccatcctttgggacgccatcagtacctatgatggctcgaaggctctccatgtcaccaacatcaaaaaatggaaggagccctgccgaatagaactctacagagtcgtagagagtttagccaaggcacaggagacatcaggagaagaaatttccaaattttacctgccaaactgcaacaagaatggattttatcacagcagacagtgtgagacatccatggatggagaggcgggactctgctggtgcgtctacccttggaatgggaagaggatccctgggtctccagagatcaggggagaccccaactgccagatatattttaatgtacaaaactgaaaccagatgaaataatgttctgtcacgtgaaatatttaagtatatagtatatttatactctagaacatgcacatttatatatatatgtatatgtatatatatatagtaactactttttatactccatacataacttgatatagaaagctgtttatttattcactgtaagtttattttttctacacagtaaaaacttgtactatgttaataacttgtcctatgtcaatttgtatatcatgaaacacttctcatcatattgtatgtaagtaattgcatttctgctcttccaaagctcctgcgtctgtttttaaagagcatggaaaaatactgcctagaaaatgcaaaatgaaataagagagagtagtttttcagctagtttgaaggaggacggttaacttgtatattccaccattcacatttgatgtacatgtgtagggaaagttaaaagtgttgattacataatcaaagctacctgtggtgatgttgccacctgttaaaatgtacactggatatgttgttaaacacgtgtctataatggaaacatttacaataaatattctgcatggaaatactgttaaaaaaaaaaa

SEQ ID NO: 8 - protein, 259 aa, Homo sapiens
  misc_feature (1)..(259): insulin-like growth factor-binding protein 1 precursor
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 9 - DNA, 4629 bp, Homo sapiens
  misc_feature (1)..(4629): Homo sapiens adiponectin, C1Q and collagen domain containing (ADIPOQ), transcript variant 1, mRNA
aggctgttgaggctgggccatctcctcctcacttccattctgactgcagtctgtggttctgattccataccagaggagacgggatttcaccatgttgtccaggctggtctgaaactcctgacatcagggctcaggatgctgttgctgggagctgttctactgctattagctctgcccggtcatgaccaggaaaccacgactcaagggcccggagtcctgcttcccctgcccaagggggcctgcacaggttggatggcgggcatcccagggcatccgggccataatggggccccaggccgtgatggcagagatggcacccctggtgagaagggtgagaaaggagatccaggtcttattggtcctaagggagacatcggtgaaaccggagtacccggggctgaaggtccccgaggctttccgggaatccaaggcaggaaaggagaacctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactgatcaccactaactcagagcctcctccaggccaaacagccccaaagtcaattaaaggctttcagtacggttaggaagttgattattatttagttggaggcctttagatattattcattcatttactcattcatttattcattcattcatcgagtaactttaaaaaaatcatatgctatgttcccagtcctggggagcttcacaaacatgaccagataactgactagaaagaagtagttgacagtgctattttgtgcccactgtctctcctgatgctcatatcaatcctataaggcacagggaacaagcattctcctgtttttacagattgtatcctgaggctgagagagttaagtgaatgtctaaggtcacacagtattaagtgacagtgctagaaatcaaacccagagctgtggactttgttcactagactgtgcccttttatagaggtacatgttctctttggagtgttggtaggtgtctgtttcccacctcacctgagagccattgaatttgccttcctcatgaattaaaacctcccccaagcagagcttcctcagagaaagtggttctatgatgacgtcctgtcttggaaggactactactcaatggcccctgcactactctacttcctcttacctatgtcccttctcatgcctttccctccaacggggaaagccaactccatctctaagtgccgaactcatccctgttcctcaaggccacctggccaggagcttctctgatgtgatatccactttttttttttttgagatggagtctcactctgtcacccaggctggagtacagtgacacgacctcggctcactgcagcctccttctcctgggtccaagcaattattgtgcctcagcctcccgagtagctgagacttcaggtgcattccaccacacatggctaatttttgtatttttagtagaaatggggtttcgtcatgttggccaggctggtctcgaactcctggcctaggtgatccacccgcctcgacctcccaaagtgctgggattacaggcatgagccaccatgcccagtcgatatctcactttttattttgccatggatgagagtcctgggtgtgaggaacacctcccaccaggctagaggcaactgcccaggaaggactgtgcttccgtcacctctaaatcccttgcagatccttgataaatgcctcatgaagaccaatctcttgaatcccatatctacccagaattaactccattccagtctctgcatgtaatcagttttatccacagaaacattttcattttaggaaatccctggttttaagtatcaatccttgttcagctggacaatatgaatcttttccactgaagttagggatgactgtgattttcagaacacgtccagaatttttcatcaagaaggtagcttgagcctgaaatgcaaaacccatggaggaattctgaagccattgtctccttgagtaccaacagggtcagggaagactgggcctcctgaatttattattgttctttaagaattacaggttgaggtagttgatggtggtaaacattctctcaggagacaataactccagtgatgttcttcaaagattttagcaaaaacagagtaaatagcattctctatcaatatataaatttaaaaaactatctttttgcttacagttttaaattctgaacaattctctcttatatgtgtattgctaatcattaaggtattattttttccacatataaagctttgtctttttgttgttgttgttgtttttaagatggagtttccctctgttgccaggctagagtgcagtggcatgatctcggcttactgcaacctttgcctcccaggttcaagcgattcttctgcctcagcctcccgagtagctgggaccacaggtgcctaccaccatgccaggctaatttttgtatttttagtaaagacagggtttcaccatattggccaggctggtctcgaactcctgaccttgtgatctgcccgcctccatttttgttgttattttttgagaaagatagatatgaggtttagagagggatgaagaggtgagagtaagccttgtgttagtcagaactctgtgttgtgaatgtcattcacaacagaaaacccaaaatattatgcaaactactgtaagcaagaaaaataaaggaaaaatggaaacatttattcctttgcataatagaaattaccagagttgttctgtctttagataaggtttgaaccaaagctcaaaacaatcaagacccttttctgtatgtccttctgttctgccttccgcagtgtaggctttaccctcaggtgctacacagtatagttctagggtttccctcccgatatcaaaaagactgtggcctgcccagctctcgtatccccaagccacaccatctggctaaatggacatcatgttttctggtgatgcccaaagaggagagaggaagctctctttcccagatgccccagcaagtgtaaccttgcatctcattgctctggctgagttgtgtgcctgtttctgaccaatcactgagtcaggaggatgaaatattcatattgacttaattgcagcttaagttaggggtatgtagaggtattttccctaaagcaaaattgggacactgttatcagaaataggagagtggatgatagatgcaaaataatacctgtccacaacaaactcttaatgctgtgtttgagctttcatgagtttcccagagagacatagctggaaaattcctattgattttctctaaaatttcaacaagtagctaaagtctggctatgctcacagtctcacatctggttggggtgggctccttacagaacacgctttcacagttaccctaaactctctggggcagggttattcctttgtggaaccagaggcacagagagagtcaactgaggccaaaagaggcctgagagaaactgaggtcaagatttcaggattaatggtcctgtgatgctttgaagtacaattgtggatttgtccaattctctttagttctgtcagcttttgcttcatatattttagcgctctattattagatatatacatgtttagtattatgtcttattggtgcatttactctcttatcattatgtaatgtccttctttatctgtgataattttctgtgttctgaagtctactttgtctaaaaataacatacgcactcaacttccttttctttcttccttcctttctttcttccttcctttctttctctctctctctctttccttccttccttcctccttttctttctctctctctctctctctctttttttgacagactctcgttctgtggccctggctggagttcagtggtgtgatcttggctcactgctacctctaccatgagcaattctcctgcctcagcctcccaagtagctggaactacaggctcatgccactgcgcccagctaatttttgtatttttcgtagagacggggtttcaccacattcgtcaggttggtttcaaactcctgactttgtgatccacccgcctcggcctcccaaagtgctgggattacaggcatgagccatcacacctggtcaactttcttttgattagtgtttttgtggtatatctttttccatcatgttactttaaatatatctatattattgtatttaaaatgtgtttcttacagactgcatgtagttgggtataatttttatccagtctaaaaatatctgtcttttaattggtgtttagacaatttatatttaataaaattgttgaatttaa

SEQ ID NO: 10 - protein, 244 aa, Homo sapiens
  misc_feature (1)..(244): adiponectin precursor
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 11 - DNA, 1294 bp, Homo sapiens
  misc_feature (1)..(1294): Homo sapiens glucagon (GCG), mRNA
gcatagaatgcagatgagcaaagtgagtgggagagggaagtcatttgtaacaaaaactcattatttacagatgagaaatttatattgtcagcgtaatatctgtgaggctaaacagagctggagagtatataaaagcagtgcgccttggtgcagaagtacagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttctttaccattgtct

SEQ ID NO: 12 - protein, 180 aa, Homo sapiens
  misc_feature (1)..(180): glucagon preproprotein
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 13 - DNA, 1333 bp, Homo sapiens
  misc_feature (1)..(1333): Homo sapiens sex hormone-binding globulin (SHBG), transcript variant 1, mRNA
accgcccacacgcaaggctgcctgcctctacacattctcccaagagttgtctgagccgccgagtggacagtggctgattatggagagcagaggcccactggctacctcgcgcctgctgctgttgctgctgttgctactactgcgtcacacccgccagggatgggccctgagacctgttctccccacccagagtgcccacgaccctccggctgtccacctcagcaatggcccaggacaagagcctatcgctgtcatgacctttgacctcaccaagatcacaaaaacctcctcctcctttgaggttcgaacctgggacccagagggagtgattttttatggggataccaaccctaaggatgactggtttatgctgggacttcgagacggcaggcctgagatccaactgcacaatcactgggcccagcttacggtgggtgctggaccacggctggatgatgggagatggcaccaggtggaagtcaagatggagggggactctgtgctgctggaggtggatggggaggaggtgctgcgcctgagacaggtctctgggcccctgaccagcaaacgccatcccatcatgaggattgcgcttggggggctgctcttccccgcttccaaccttcggttgccgctggttcctgccctggatggctgcctgcgccgggattcctggctggacaaacaggccgagatctcagcatctgcccccactagcctcagaagctgtgatgtagaatcaaatcccgggatatttctccctccagggactcaggcagaattcaatctccgagacattccccagcctcatgcagagccctgggccttctctttggacctgggactcaagcaggcagcaggctcaggccacctccttgctcttgggacaccagagaacccatcttggctcagtctccacctccaagatcaaaaggtggtgttgtcttctgggtcggggccagggctggatctgcccctggtcttgggactccctcttcagctgaagctgagtatgtccagggtggtcttgagccaagggtcgaagatgaaggcccttgccctgcctcccttaggcctggctcccctccttaacctctgggccaagcctcaagggcgtctcttcctgggggctttaccaggagaagactcttccacctctttttgcctgaatggcctttgggcacaaggtcagaggctggatgtggaccaggccctgaacagaagccatgagatctggactcacagctgcccccagagcccaggcaatggcactgacgcttcccattaaagctccacctaagaaccccctttgaaagttaaaaaaaaaaaaaaa

SEQ ID NO: 14 - protein, 402 aa, Homo sapiens
  misc_feature (1)..(402): sex hormone-binding globulin isoform 1 precursor
MESRGPLATSRLLLLLLLLLLRHTRQGWALRPVLPTQSAHDPPAVHLSNGPGQEPIAVMTFDLTKITKTSSSFEVRTWDPEGVIFYGDTNPKDDWFMLGLRDGRPEIQLHNHWAQLTVGAGPRLDDGRWHQVEVKMEGDSVLLEVDGEEVLRLRQVSGPLTSKRHPIMRIALGGLLFPASNLRLPLVPALDGCLRRDSWLDKQAEISASAPTSLRSCDVESNPGIFLPPGTQAEFNLRDIPQPHAEPWAFSLDLGLKQAAGSGHLLALGTPENPSWLSLHLQDQKVVLSSGSGPGLDLPLVLGLPLQLKLSMSRVVLSQGSKMKALALPPLGLAPLLNLWAKPQGRLFLGALPGEDSSTSFCLNGLWAQGQRLDVDQALNRSHEIWTHSCPQSPGNGTDASH

SEQ ID NO: 15 - DNA, 1166 bp, Homo sapiens
  misc_feature (1)..(1166): Homo sapiens chemokine (C-X-C motif) ligand 3 (CXCL3), mRNA
gctccgggaatttccctggcccggccgctccgggctttccagtctcaaccatgcataaaaagggttcgccgatcttggggagccacacagcccgggtcgcaggcacctccccgccagctctcccgcttctcgcacagcttcccgacgcgtctgctgagccccatggcccacgccacgctctccgccgcccccagcaatccccggctcctgcgggtggcgctgctgctcctgctcctggtggccgccagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaaggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtggggggattgaaatgcaagcaattagtggatcactgttagggtaagggaatgtatgtacacatctattttttatactttttttttaaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatgaaaaaaaaaaaaaaa

SEQ ID NO: 16 - protein, 107 aa, Homo sapiens
  misc_feature (1)..(107): C-X-C motif chemokine 3 precursor
MAHATLSAAPSNPRLLRVALLLLLLVAASRRAAGASVVTELRCQCLQTLQGIHLKNIQSVNVRSPGPHCAQTEVIATLKNGKKACLNPASPMVQKIIEKILNKGSTN

SEQ ID NO: 17 - DNA, 1234 bp, Homo sapiens
  misc_feature (1)..(1234): Homo sapiens chemokine (C-X-C motif) ligand 2 (CXCL2), mRNA
gagctccgggaatttccctggcccgggactccgggctttccagccccaaccatgcataaaaggggttcgccgttctcggagagccacagagcccgggccacaggcagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgcccttacaggaacagaagaggaaagagagacacagctgcagaggccacctggattgcgcctaatgtgtttgagcatcacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtgagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatataaaaaaaaaaaaaaaaaaaaaaaaaaaaaaaaa

SEQ ID NO: 18 - protein, 107 aa, Homo sapiens
  misc_feature (1)..(107): C-X-C motif chemokine 2 precursor
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 19 - DNA, 994 bp, Homo sapiens
  misc_feature (1)..(994): Homo sapiens tumor necrosis factor receptor superfamily, member 17 (TNFRSF17), Mrna
aagactcaaacttagaaacttgaattagatgtggtattcaaatccttagctgccgcgaagacacagacagcccccgtaagaacccacgaagcaggcgaagttcattgttctcaacattctagctgctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtgtgaccaattcagtgaaaggaacgaatgcgattctctggacctgtttgggactgagcttaataatttctttggcagttttcgtgctaatgtttttgctaaggaagataaactctgaaccattaaaggacgagtttaaaaacacaggatcaggtctcctgggcatggctaacattgacctggaaaagagcaggactggtgatgaaattattcttccgagaggcctcgagtacacggtggaagaatgcacctgtgaagactgcatcaagagcaaaccgaaggtcgactctgaccattgctttccactcccagctatggaggaaggcgcaaccattcttgtcaccacgaaaacgaatgactattgcaagagcctgccagctgctttgagtgctacggagatagagaaatcaatttctgctaggtaattaaccatttcgactcgagcagtgccactttaaaaatcttttgtcagaatagatgatgtgtcagatctctttaggatgactgtatttttcagttgccgatacagctttttgtcctctaactgtggaaactctttatgttagatatatttctctaggttactgttgggagcttaatggtagaaacttccttggtttcatgattaaactcttttttttcctga

SEQ ID NO: 20 - protein, 184 aa, Homo sapiens
  misc_feature (1)..(184): tumor necrosis factor receptor superfamily member 17
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 21 - DNA, 1957 bp, Homo sapiens
  misc_feature (1)..(1957): Homo sapiens adhesion molecule, interacts with CXADR antigen 1 (AMICA1), transcript variant 1, Mrna
ctgtttgcatctctgcaaccacttcagaaggcacgtgtttggtttgctctgagcctaacctagagtgctcgcagcagtctttcagttgagcttggggactgcagctgtggggagatttcagtgcattgcctcccctgggtgctcttcatcttggatttgaaagttgagagcagcatgttttgcccactgaaactcatcctgctgccagtgttactggattattccttgggcctgaatgacttgaatgtttccccgcctgagctaacagtccatgtgggtgattcagctctgatgggatgtgttttccagagcacagaagacaaatgtatattcaagatagactggactctgtcaccaggagagcacgccaaggacgaatatgtgctatactattactccaatctcagtgtgcctattgggcgcttccagaaccgcgtacacttgatgggggacatcttatgcaatgatggctctctcctgctccaagatgtgcaagaggctgaccagggaacctatatctgtgaaatccgcctcaaaggggagagccaggtgttcaagaaggcggtggtactgcatgtgcttccagaggagcccaaagagctcatggtccatgtgggtggattgattcagatgggatgtgttttccagagcacagaagtgaaacacgtgaccaaggtagaatggatattttcaggacggcgcgcaaaggaggagattgtatttcgttactaccacaaactcaggatgtctgtggagtactcccagagctggggccacttccagaatcgtgtgaacctggtgggggacattttccgcaatgacggttccatcatgcttcaaggagtgagggagtcagatggaggaaactacacctgcagtatccacctagggaacctggtgttcaagaaaaccattgtgctgcatgtcagcccggaagagcctcgaacactggtgaccccggcagccctgaggcctctggtcttgggtggtaatcagttggtgatcattgtgggaattgtctgtgccacaatcctgctgctccctgttctgatattgatcgtgaagaagacctgtggaaataagagttcagtgaattctacagtcttggtgaagaacacgaagaagactaatccagagataaaagaaaaaccctgccattttgaaagatgtgaaggggagaaacacatttactccccaataattgtacgggaggtgatcgaggaagaagaaccaagtgaaaaatcagaggccacctacatgaccatgcacccagtttggccttctctgaggtcagatcggaacaactcacttgaaaaaaagtcaggtgggggaatgccaaaaacacagcaagccttttgagaagaatggagagtcccttcatctcagcagcggtggagactctctcctgtgtgtgtcctgggccactctaccagtgatttcagactcccgctctcccagctgtcctcctgtctcattgtttggtcaatacactgaagatggagaatttggagcctggcagagagactggacagctctggaggaacaggcctgctgaggggaggggagcatggacttggcctctggagtgggacactggccctgggaaccaggctgagctgagtggcctcaaaccccccgttggatcagaccctcctgtgggcagggttcttagtggatgagttactgggaagaatcagagataaaaaccaacccaaatcattcctctggcacattatttctaaataagtgttgtttgtggaaatcgtcctatttttatgcctcggttgagggggcaagtaaagtcctggtgaggaatttacagttagaaacttttggaaaaactgaagttcctggaggaaagccaaaccgtgctaacacactagaggctggttttggtcctgggaaaggtttggggtgaatgggttggaagtagggtatgaatacagttttgggattt

SEQ ID NO: 22 - protein, 394 aa, Homo sapiens
  misc_feature (1)..(394): junctional adhesion molecule-like isoform 1 precursor
MFCPLKLILLPVLLDYSLGLNDLNVSPPELTVHVGDSALMGCVFQSTEDKCIFKIDWTLSPGEHAKDEYVLYYYSNLSVPIGRFQNRVHLMGDILCNDGSLLLQDVQEADQGTYICEIRLKGESQVFKKAVVLHVLPEEPKELMVHVGGLIQMGCVFQSTEVKHVTKVEWIFSGRRAKEEIVFRYYHKLRMSVEYSQSWGHFQNRVNLVGDIFRNDGSIMLQGVRESDGGNYTCSIHLGNLVFKKTIVLHVSPEEPRTLVTPAALRPLVLGGNQLVIIVGIVCATILLLPVLILIVKKTCGNKSSVNSTVLVKNTKKTNPEIKEKPCHFERCEGEKHIYSPIIVREVIEEEEPSEKSEATYMTMHPVWPSLRSDRNNSLEKKSGGGMPKTQQAF

SEQ ID NO: 23 - DNA, 1054 bp, Homo sapiens
  misc_feature (1)..(1054): Homo sapiens trefoil factor 3 (intestinal) (TFF3), mRNA
gccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtgggagagggcgggagggtgtcaggggagagtctgcccaggcctcaagggcaggaaaagactccctaaggagctgcagtgcatgcaaggatattttgaatccagactggcacccacgtcacaggaaagcctaggaacactgtaagtgccgcttcctcgggaaagcagaaaaaatacatttcaggtagaagttttcaaaaatcacaagtctttcttggtgaagacagcaagccaataaaactgtcttccaaagtggtcctttatttcacaaccactctcgctactgttcaatacttgtactattcctgggttttgtttctttgtacagtaaacattatgaacaaacaggca

SEQ ID NO: 24 - protein, 94 aa, Homo sapiens
  misc_feature (1)..(94): trefoil factor 3 precursor
MKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 25 - DNA, 3236 bp, Homo sapiens
  misc_feature (1)..(3236): Homo sapiens ephrin-B3 (EFNB3), Mrna
ggtttcctcccttagcccgctgccctcaatcccagcgaggctggggctccggctcggcgcccccttcctcgctccctggtccggcgccccatgccgcccccgcccggtccccggctcccccagtcccccacttaggcgggctcacagatcccggggtgctggcgcgtgggccgggggcgcgtagggcgcctgcagacggcccctggaagggctctggtggggctgagcgctctgccgcgggggcgcgggcacagcaggaagcaggtccgcgtgggcgctgggggcatcagctaccggggtggtccgggctgaagagccaggcagccaaggcagccaccccggggggtgggcgactttgggggagttggtgccccgccccccaggccttggcggggtcatggggcccccccattctgggccggggggcgtgcgagtcggggccctgctgctgctgggggttttggggctggtgtctgggctcagcctggagcctgtctactggaactcggcgaataagaggttccaggcagagggtggttatgtgctgtaccctcagatcggggaccggctagacctgctctgcccccgggcccggcctcctggccctcactcctctcctaattatgagttctacaagctgtacctggtagggggtgctcagggccggcgctgtgaggcaccccctgccccaaacctccttctcacttgtgatcgcccagacctggatctccgcttcaccatcaagttccaggagtatagccctaatctctggggccacgagttccgctcgcaccacgattactacatcattgccacatcggatgggacccgggagggcctggagagcctgcagggaggtgtgtgcctaaccagaggcatgaaggtgcttctccgagtgggacaaagtccccgaggaggggctgtcccccgaaaacctgtgtctgaaatgcccatggaaagagaccgaggggcagcccacagcctggagcctgggaaggagaacctgccaggtgaccccaccagcaatgcaacctcccggggtgctgaaggccccctgccccctcccagcatgcctgcagtggctggggcagcaggggggctggcgctgctcttgctgggcgtggcaggggctgggggtgccatgtgttggcggagacggcgggccaagccttcggagagtcgccaccctggtcctggctccttcgggaggggagggtctctgggcctggggggtggaggtgggatgggacctcgggaggctgagcctggggagctagggatagctctgcggggtggcggggctgcagatccccccttctgcccccactatgagaaggtgagtggtgactatgggcatcctgtgtatatcgtgcaggatgggcccccccagagccctccaaacatctactacaaggtatgagggctcctctcacgtggctatcctgaatccagcccttcttggggtgctcctccagtttaattcctggtttgagggacacctctaacatctcggccccctgtgcccccccagccccttcactcctcccggctgctgtcctcgtctccacttttaggattccttaggattcccactgccccacttcctgccctcccgtttggccatgggtgcccccctctgtctcagtgtccctggatcctttttccttggggaggggcacaggctcagcctcctctctgaccatgacccaggcatccttgtccccctcacccacccagagctaggggcgggaacagcccaccttttggttggcaccgccttctttctgcctctcactggttttctcttctctatctcttattctttccctctcttccgtctctaggtctgttcttcttccctagcatcctcctccccacatctcctttcaccctcttggcttcttatcctgtgcctctcccatctcctgggtgggggcatcaaagcatttctccccttagctttcagccccccttctgacctctcataccaaccactcccctcagtctgccaaaaatgggggccttatggggaaggctctgacactccaccccagctcaggccatgggcagcagggctccattctctggcctggcccaggcctctacatacttactccagccatttggggtggttgggtcatgacagctaccatgagaagaagtgtcccgttttgtccagtggccaatagcaagatatgaaccggtcgggacatgtatggacttggtctgatgctgaatgggccacttgggaccggaagtgacttgctccagacaagaggtgaccaggcccggacagaaatggcctgggaagtagcagaagcagtgcagcaggaactggaagtgccttcatccaggacaggaagtagcacttctgaaacaggaagtggtctggctggaactccaagtggcttagtctgggggatcaggaggtgggaggtggatggttcttattctgtggagaagaagggcgggaagaacttcctttcaggaggaagctggaacttactgactgtaagaggttagaggtggaccgagaaggacttttcccagtcttcagtggcacttcccaagatctcccttcccttgtgctctgtgctgattttaggacagctaagatgactgccatgtgctgtggcaggcctaatttgtcttgttctttcctttccatatcccagtataatctctgttaatcaacaggactaccccaagaacccatgtgctctcccgagtaacccagatggctgtcttgttcattccatcctacatttctgactcctttcagactcaacacagttcccttcttagtgaccaaaatggtggcctactggctggtctagctgacagtggtacttagcaaaggccactgtttccatagtgaccagctgatacctcttcctgccctctagtgtgcaattgggtgttgcctcagtttcctcccagctcagttttattagatcaaagctgttgttgggcaccaggttggccacctcaatcaccagccaagatggttgctttgtccaccagaggtcaagttcacctctctggtgctgtagttcccagctccttcctgatttttctaatcgctccttctggggaacaggaagttgatattgccatggtggcggggtatgccgtcacctcagtagttttactgtaaaagggaaatttgaacaacaaaaaccaaaaaaaataaaaataaaaaacttcaaaagttgacaaaaaaaaaaaaaaaaaaa

SEQ ID NO: 26 - protein, 340 aa, Homo sapiens
  misc_feature (1)..(340): ephrin-B3 precursor
MGPPHSGPGGVRVGALLLLGVLGLVSGLSLEPVYWNSANKRFQAEGGYVLYPQIGDRLDLLCPRARPPGPHSSPNYEFYKLYLVGGAQGRRCEAPPAPNLLLTCDRPDLDLRFTIKFQEYSPNLWGHEFRSHHDYYIIATSDGTREGLESLQGGVCLTRGMKVLLRVGQSPRGGAVPRKPVSEMPMERDRGAAHSLEPGKENLPGDPTSNATSRGAEGPLPPPSMPAVAGAAGGLALLLLGVAGAGGAMCWRRRRAKPSESRHPGPGSFGRGGSLGLGGGGGMGPREAEPGELGIALRGGGAADPPFCPHYEKVSGDYGHPVYIVQDGPPQSPPNIYYKV

SEQ ID NO: 27 - DNA, 9478 bp, Homo sapiens
  misc_feature (1)..(9478): Homo sapiens limbic system-associated membrane protein (LSAMP), mRNA
ggaggagggggagagaggctctgggttgctgctgcttctgctgctgctgctgctgtgtggctgtttctgtacactcactggcaggcttggtgccggctccctcgcccgcccgcccgccagcctgggaaagtgggttacagagcgaaggagctcagctcagacactggcagaggagcatccagtcacagagagaccaaacaagaaccctttcctttggcttcctcttcagctcttccagagggcttgctatttgcactctctcttttgaaattgtgttgcttttacttttcacccttctgcttgggttttatgagggctttgttaagtcttagagggaaaagagactgagcgagggaaagagagaggcaaagtggaaaggaccataaactggcaaagcccgctctgcgctcgctgtggatgaaagccccgtgttggtgaagcctctcctcgcgagcagcgcgcacccctccagagcaccccgcggacccgcacctcggcgtggccaccatggtcaggagagttcagccggatcggaaacagttgccactggtcctactgagattgctctgccttcttcccacaggactgcctgttcgcagcgtggattttaaccgaggcacggacaacatcaccgtgaggcagggggacacagccatcctcaggtgcgttgtagaagacaagaactcaaaggtggcctggttgaaccgttctggcatcatttttgctggacatgacaagtggtctctggacccacgggttgagctggagaaacgccattctctggaatacagcctccgaatccagaaggtggatgtctatgatgagggttcctacacttgctcagttcagacacagcatgagcccaagacctcccaagtttacttgatcgtacaagtcccaccaaagatctccaatatctcctcggatgtcactgtgaatgagggcagcaacgtgactctggtctgcatggccaatggccgtcctgaacctgttatcacctggagacaccttacaccaactggaagggaatttgaaggagaagaagaatatctggagatccttggcatcaccagggagcagtcaggcaaatatgagtgcaaagctgccaacgaggtctcctcggcggatgtcaaacaagtcaaggtcactgtgaactatcctcccactatcacagaatccaagagcaatgaagccaccacaggacgacaagcttcactcaaatgtgaggcctcggcagtgcctgcacctgactttgagtggtaccgggatgacactaggataaatagtgccaatggccttgagattaagagcacggagggccagtcttccctgacggtgaccaacgtcactgaggagcactacggcaactacacctgtgtggctgccaacaagctgggggtcaccaatgccagcctagtccttttcagacctgggtcggtgagaggaataaatggatccatcagtctggccgtaccactgtggctgctggcagcatctctgctctgccttctcagcaaatgttaatagaataaaaatttaaaaataatttaaaaaacacacaaaaatgcgtcacacagaatacagagagagagagacagagagagagagagagagagagatgggggagaccgtttatttcacaactttgtgtgtttatacatgaagggggaaataagaaagtgaagaagaaaatacaacatttaaaacaattttacagtccatcattaaaaatttatgtatcattcaggatggagaaggttctactgggatatgtttatatctactaagcaaatgtatgctgtgtaaagactacaccacactaaggacatctggatgctgtaaaaataagagaagaaccagatggatattaagccccccaacacacactttatccttccttccttcatcttttttcatctgtggggaagaaaataaggtcttgcctttggtgtttatatttccataaccttttaattctatttttcatttgagctgacttgtagccacttcagactatcaatggaatcttatgttgagcctttctctggctttccttcctccactatctctccaactttagagatcatcccctctccctccagtgcgttctatctcccccacacccaccctagatactcccttttcacccacctttcctccctcacctctcctcacctccaccccctccccagagcactagtcatgccgcaaatgctaggaagtgccattttcattttctccactgtgcgtgtgtgctcaagtctttcgctctcacgtgggtgtacatgtgtgtgagcgtgtgtgtgtctctctctaaagcatgccaagggaatggtccatgtgtacatagactcattgtgctgtagatactgtcctgcattgtaattgtgagatgcggctgtaacaagttgctgggggagatggcggggaaagaggcaaggagcagagtcctccctacatccatggctgtcacatggcatcagtgtgtattcaaaccaagctatgctccttccaagggcaggaccccatattcctcctagtcccatcatcagaaccgagtggggagtcactcagaatatcactgtaaatgaaagtgcctactatcgatggggtaagcaaacagcataaggaattatgacgtggacgaggtgacctaggagagaaaatttcagattttactctcatttcatgagtctgagggattcttatatttcctggcatttaacagggtaggccctgctccactgtgaaaatgagcagcatgtgttgagtaaatccccagaaacaggaaggtctccaagtgtcaactcccagtgaaagaatgatgaaccacttggagatcctaagcagccctgttttacctcctccctaatcttaaataacatttgtcccatgaattcccctgagcagagattgtttcctatttcagataaaatacagtgaaagtgagcaaggcagaaaaagtcaacagatgcccaggctcctactgtattctggagatactgtcagagctctaatacagagcactggccataatgaaaagcagttcactccttgtgctcctctgcagatgtttttcccagtgttctaggttaatgttttatttggttgcctgcataatccctgttctgtttcactgatggtgtttgcagcaccactgttcatggtggtccactgttatcctatgccagggtgctaagaattgcatgatattcatcttccctgctctatttaaatttacatctataagagtcatcttgacattaacactgaaatgtgatctaggtccttaaccaaaattgctgggcaacttgtaataaatttagacagaaattttatgagtaccacaaagcttggtgttaccacatcaccagaaggatttcttaggaaatgtcttgccgagagagctggctctctgcatatagatgtctttgtcagaaaaccaacccttgctctcacttacacagtagtaagcactgaaagtggttcagttcatgagaggacagagaattattttgagattatatttgaatgtaatcttgcagagccaaatatggtatgtcattaagttggaaccttgtaaatagctgttccatgttataaaatgagaaactttgtaactggaaaaaaagaaaggaaagaaggaaggaaggaaggaaggaaggcagggagggggggacggggaagggggggagggagggagggagggagggagggagggagggagggaggaaggaaggaaggaaggaaggaaggagaaaggaaaggaaggcaggagggagaaagatctaagtagcattgttaatttcttcaatttcttctaagggattttattgtttgttttagaagcttatcacagccttctttcatgattttgcagtttagacttgatacaaggaaaaattcagcttggggatggttaagagtgtttatagcagattctgacataggagagaaaacaaattctcatccaagaaggtagctagtaaaatatagggaaggtgagccatattcctatgcagcatcaatttattgacaatcaggtatttctcttaacagtttggtcttcttagttcaagaataaagggtatcatctttaataataagcattccccaaaaattgaagaggcagtcacacacttaagtgtgtggctttagaaaagcgcatgctaatttaaagatatacaggaagagaaaagtaggagttaagttggatgttgttagaagttggatgttagtattaccttcaggaacagatccccatggcatgtcacaggccttaattatatacctggctttcttattgtctccactttatcatgaggacaaggtcttggtttcatgggaggaacttctccattgaaataaatgtctgccatgtcagcaccgtttgttccctcagttttaatataatggaccatatattaaacataattaaacatatatttaaatgtggtgtttgcctgtgtctctagcaggatcttgaaattttaaaaatttgcttctggttcctgtttcagagaaaacattgtccccagaaatttcataggattgaaagtgttccctaagcagtgtgaacaatggaggaaaatatagtttagagaaaagtcagggaaaggtagggccagaggactgacaccaagaaatcattgaatctcaacataagacttcttggaatttagttaatcatattggaataaattccttcaagaatcttgtcccttggtaatcaaagtttgaaaccccgcactgaaaagcaccaactggttggaaataatatactgagaggagtgaaattcatcaattaatctgagtggctaatatatttaatatcctttgtatacaaagtaaaactccaccattcgtaaaaggaaatccttagacccaactttcagttaacaaaaacagaaatgactttgacccagggtgcttcctgaagaatgagaactatccagggctttacaactgcagaattgtaattatgctctgtgcaattgttgagcaaaggttttgccttgctggataaaaagtcttgtttgtttcgagacatgaaatccccatgtcttaaaagaactaaggcttatagaaaagcagatgggttttctctcaggaaggactgccccattgacctttgccttctcttccaagtcagacagacttctcgcttgccatgggcatttttttactacatagtcagactactggggctacttatagagaccttgtaaaagtactcgtgattttcacgttcttggaggaccaaacaaaaatctgtttctcctccaaaaatggacttacctcctttgcacacaaaagctaaactcctcagcatgaaattgtttgagttattactttaccaagttgtgagcttcttgaatcctccagagtcgcagattccatccctgagttggttgtggtttcactgtttctattggctattctccctgaatttttcattttgttctttgcagggctcgaattatttgtggaaaacaataatatatatgtgtgtgtattttttatctttatagatgctatatttacaataatgtatgtattataagacaaattaagaataatgttttgatcttaaaaggaagaaaagtactgaatttggttgtttagaaagaaaatctatgctcacgtagaaagacatagagccccactttttccgttttgtaattatttggcgaaaagaaatttgcttatagactatgtttaatgggattaaccatgtcctcatttttcttttcatcctcatacacttttagcctgcatttagtcttggttaccaatatcattttttaagagaaatgtaagtacagtgctatatcttacctacataaactattaatattttgaagacaaatgtgaaacacaccacaaaaatggtgagataagaaacaaaaatgcagtttaggaagcctcctccttgcttaaatgtttagaatatttcttctccaaagactgcatttgcctcagtgatgtaaattttccatcatggttggcataatatctgtaaacatctcactaaatgcaaaatggagtttacatttatgtgcatactagcagaaaagaagtaaactattctcatttgcatgtagctatgctgttcaaatgtcgccaaccaaaatttaggaaagaatttgttttcacccagcatgtacatctcacttttctcttggcaaggaagctggtgttaacgttgggtttaggtttaacatttacaccagcgaaaatgtttatgaatattatggaaaacttattttaaaccttgatttcttttgagcacatttacatgctgcgtgctgattaataaattaggcaccaatcatgtgtaaatcaatgtaaatcactagtttatgtacataatataaactatgtaaacttcatttttcatgcagtgcccaactactgctaaggtttactatgattcttaaggaaaaaaaaatcaaataaaaataaataaaaactgaaacgtttcacagttcagaatcggaagaattacgactaaagctccaaatatgaggttgccttagccaaaggaagcagacccacaggaacagttcaaggtttatatcctgctcaagtcacctctttggtctttcaggatctaagtggagattgtcccaactgttgctgtagttgtctcacccgaccccaaagcaagggaaatagagggaaaggttttaagggctatatgtctgctgtatccactcccagctatcttctttttactcctttctcaccatctaagatgtcttatttaaatagctccaaggaagtgacctaaaccttgatgagcaaaatattactcagtttttattttccattcaacaaaagcagtgggaaagcttgccatctggattctaagaaattgtgcaatataaaaaatgttatatcctcgagaaatatcttgctgagtcaccctaggaaataactaccttttatttatctggtagcctaatgtttccacacatttatcctgaatattgcaagtgagggactgaatcatttttaattgggagttatctttctcaggcatgttctcttggagtctttttgaagtgcctgacacgtgtaacaggatgacatatatatcattcctacaatatgaacaactgttacataaaaaattatcaaggagatgatttcaggaaacaagtgaactttctgcaaagacttataaaaattttaggtcaaattaacttcaggctttaaatgcactaatctacctaagagaaaaaaaaagaaaaaaaacaggaaggagttttaagggctcatgtgcctgttgtatcaacccccaagtgtcttcttggtactgcttcctcatcatctaagataaactgacaactttaaagtgaggtagaaggtgtattgaattgggagtcaggagaattgggttctaaccccaagttcagccacaaataaagctgtgagacattggcaagtcatttaactttcctgagtcttggttttctcatcctgaaagtgagggattcggctgacgtctctaaaatctctttcaactctaacctttattctgaataagaatttaatattcacttagtgctgtgcccagcactgtttgtaaacagcagctgtttgttatctctagtttggtctctgtattctcatcacttctcagaactatcattctaccgtcttcatttctaaacccaaaactgctagaatacagggactctggactgggtctgtaaattttttctgatcaaaactttatagcagtgtagagaagggacacattcaaattacactaaggacattgacatagctggggttgtttccttgtttatattataaaacctaaatgtggaactatattctaataatctttcataggaaggaaaatagccagactgggtattatgcatgtaacaaatgaggacattgtgcataagaaaggaaacattagttttctgtcatcctgggccaagtacctcattacagtaaatgtgtgtctttggaaactctttgcttgtgctgatggcggtaagcatggggtcccaggcaggttcaaaggctgaactgtaagaaatgggcaagacaatacattttgttttggaaggaatttctcatgggataagtttcccaaagcttgaattataggctatgaaataaagcaaatagatggagagaaaacaagtattgttttcaaaaagtacaagtcaattctatttaaagaagacaagctgaaaataaaacaaaaataaacacaatttaggaggttacagagttgaagacagtatgaattgttgtgaaggccaaaatcaaatgtgaaagttaggttctctgagaaaagggtaagcagaaaggatgatttctcaagcaattaataaggaattattttcttgtgccatgttctagatgcattgagcacagatcctcttgtcctaagctgtcctagaggctcaggttagcatctatccaaagttgtcctttgattttattgtctgaaagaacagaaggcatcagagtttccagtcactgaagagtagggtttgttcatcacttcccagcaatcacatcactttgtgtaggtaaggatatatgatgtgcttagattacttatgaagctctctctaagtgggagaatgacctgtccatgggacaactccccgttttcatggtcatttcagaagtacctctttttgggcagtgctcctggatctacttctacagccacattctactctgcacaatcctccctatgtaaagccaggcacagtacaaatatgcttcttgcaagtgaagaaaacccatggaagtcctagcttcatggcacgctgcagcaatcccaagctaccaggagcctcttttgaacccacttccctaagtctttgctcttcaccagagaatggaaattgttcatcctggtgaactgtggccaagttctgctccctaagtatttacttggagtagggaggttaaagggaagaaattcagggggagagaagcaaaagagaacacttccaactccctcccccatctcccaatgctccccaccttccttatcactgctctactgaagggtgtataaatcctgctcttggttagaattctccttattaacagtgttatatacatataaatatatatataaatatattccttttttcagccctgtagacatgaactgatcttcccttgaagatacaaacacatggccattttttgtttgggattttttgtttttcaaggtttttcatttttgtttattaggtggatttttttccctgggtactagctctgtgaaggagataaaaagcgcaatgtgtgttaaaaaaaaaaaattaaaattaaaatgaaaaaaagctttttttcttttctttttaaatgtatttaaattctgtttctctcttctgttactttacacgtatgaatgctctgctcttctgtgatcttaaaacaaaatgaaataaacgtgaaaaggagatgtgtcttcattgacctgtca

SEQ ID NO: 28 - protein, 338 aa, Homo sapiens
  misc_feature (1)..(338): limbic system-associated membrane protein preproprotein
MVRRVQPDRKQLPLVLLRLLCLLPTGLPVRSVDFNRGTDNITVRQGDTAILRCVVEDKNSKVAWLNRSGIIFAGHDKWSLDPRVELEKRHSLEYSLRIQKVDVYDEGSYTCSVQTQHEPKTSQVYLIVQVPPKISNISSDVTVNEGSNVTLVCMANGRPEPVITWRHLTPTGREFEGEEEYLEILGITREQSGKYECKAANEVSSADVKQVKVTVNYPPTITESKSNEATTGRQASLKCEASAVPAPDFEWYRDDTRINSANGLEIKSTEGQSSLTVTNVTEEHYGNYTCVAANKLGVTNASLVLFRPGSVRGINGSISLAVPLWLLAASLLCLLSKC

SEQ ID NO: 29 - protein, 11 aa, Artificial Sequence
  Synthetic peptide hypervariable region (HVR)-L1, sequence A1-A11
KASQDINSFLA

SEQ ID NO: 30 - protein, 7 aa, Artificial Sequence
  Synthetic peptide HVR-L2, sequence B1-B7
RANRLVD

SEQ ID NO: 31 - protein, 7 aa, Artificial Sequence
  Synthetic peptide HVR-L2, sequence B1-B7
RANRLVS

SEQ ID NO: 32 - protein, 7 aa, Artificial Sequence
  Synthetic peptide HVR-L2, sequence B1-B7
RANRLVE

SEQ ID NO: 33 - protein, 9 aa, Artificial Sequence
  Synthetic peptide HVR-L3, sequence C1-C9
LQYDEFPLT

SEQ ID NO: 34 - protein, 10 aa, Artificial Sequence
  Synthetic peptide HVR-H1, sequence D1-D10
GFSLTTYGVH

SEQ ID NO: 35 - protein, 17 aa, Artificial Sequence
  Synthetic peptide HVR-H2, sequence E1-E17
GVIWPGGGTDYNAAFIS

SEQ ID NO: 36 - protein, 13 aa, Artificial Sequence
  Synthetic peptide HVR-H3, sequence F1-F13
VRKEYANLYAMDY

SEQ ID NO: 37 - DNA, 2134 bp, Homo sapiens
  misc_feature (1)..(2134): Homo sapiens HGF activator (HGFAC), transcript variant 1, mRNA
gccttgaccccacctgccttggccgctctgctcccgcctcccactgcccctcaggccagctcaggagccatggggcgctgggcctgggtccccagcccctggcccccaccggggctgggccccttcctcctcctcctcctgctgctgctgctgctgccacgggggttccagccccagcctggcgggaaccgtacggagtccccagaacctaatgccacagcgacccctgcgatccccactatcctggtgacctctgtgacctctgagaccccagcaacaagtgctccagaggcagagggaccccaaagtggggggctcccgcccccgcccagggcagttccctcgagcagtagcccccaggcccaagcactcaccgaggacgggaggccctgcaggttccccttccgctacgggggccgcatgctgcatgcctgcacttcggagggcagtgcacacaggaagtggtgtgccacaactcacaactacgaccgggacagggcctggggctactgtgtggaggccaccccgcctccagggggcccagctgccctggatccctgtgcctccggcccctgcctcaatggaggctcctgctccaatacccaggacccccagtcctatcactgcagctgcccccgggccttcaccggcaaggactgcggcacagagaaatgctttgatgagacccgctacgagtacctggaggggggcgaccgctgggcccgcgtgcgccagggccacgtggaacagtgcgagtgcttcgggggccggacctggtgcgaaggcacccgacatacagcttgtctgagcagcccttgcctgaacgggggcacctgccacctgatcgtggccaccgggaccaccgtgtgtgcctgcccaccaggcttcgctggacggctctgcaacatcgagcctgatgagcgctgcttcttggggaacggcactgggtaccgtggcgtggccagcacctcagcctcgggcctcagctgcctggcctggaactccgatctgctctaccaggagctgcacgtggactccgtgggcgccgcggccctgctgggcctgggcccccatgcctactgccggaatccggacaatgacgagaggccctggtgctacgtggtgaaggacagcgcgctctcctgggagtactgccgcctggaggcctgcgatttggaaactgagggcagagaatccctcaccagagtccaactgtcaccggatctcctggcgaccctgcctgagccagcctccccggggcgccaggcctgcggcaggaggcacaagaagaggacgttcctgcggccacgtatcatcggcggctcctcctcgctgcccggctcgcacccctggctggccgccatctacatcggggacagcttctgcgccgggagcctggtccacacctgctgggtggtgtcggccgcccactgcttctcccacagcccccccagggacagcgtctccgtggtgctgggccagcacttcttcaaccgcacgacggacgtgacgcagaccttcggcatcgagaagtacatcccgtacaccctgtactcggtgttcaaccccagcgaccacgacctcgtcctgatccggctgaagaagaaaggggaccgctgtgccacacgctcgcagttcgtgcagcccatctgcctgcccgagcccggcagcaccttccccgcaggacacaagtgccagattgcgggctggggccacttggatgagaacgtgagcggctactccagctccctgcgggaggccctggtccccctggtcgccgaccacaagtgcagcagccctgaggtctacggcgccgacatcagccccaacatgctctgtgccggctacttcgactgcaagtccgacgcctgccagggggactcaggggggcccctggcctgcgagaagaacggcgtggcttacctctacggcatcatcagctggggtgacggctgcgggcggctccacaagccgggggtctacacccgcgtggccaactatgtggactggatcaacgaccggatacggcctcccaggcggcttgtggctccctcctgaccctccagcgggacaccctggttcccaccattccctgccttgctgacaataaagatatttccaagaacccggccca

SEQ ID NO: 38 - protein, 662 aa, Homo sapiens
  misc_feature (1)..(662): hepatocyte growth factor activator isoform 1 preproprotein
MGRWAWVPSPWPPPGLGPFLLLLLLLLLLPRGFQPQPGGNRTESPEPNATATPAIPTILVTSVTSETPATSAPEAEGPQSGGLPPPPRAVPSSSSPQAQALTEDGRPCRFPFRYGGRMLHACTSEGSAHRKWCATTHNYDRDRAWGYCVEATPPPGGPAALDPCASGPCLNGGSCSNTQDPQSYHCSCPRAFTGKDCGTEKCFDETRYEYLEGGDRWARVRQGHVEQCECFGGRTWCEGTRHTACLSSPCLNGGTCHLIVATGTTVCACPPGFAGRLCNIEPDERCFLGNGTGYRGVASTSASGLSCLAWNSDLLYQELHVDSVGAAALLGLGPHAYCRNPDNDERPWCYVVKDSALSWEYCRLEACDLETEGRESLTRVQLSPDLLATLPEPASPGRQACGRRHKKRTFLRPRIIGGSSSLPGSHPWLAAIYIGDSFCAGSLVHTCWVVSAAHCFSHSPPRDSVSVVLGQHFFNRTTDVTQTFGIEKYIPYTLYSVFNPSDHDLVLIRLKKKGDRCATRSQFVQPICLPEPGSTFPAGHKCQIAGWGHLDENVSGYSSSLREALVPLVADHKCSSPEVYGADISPNMLCAGYFDCKSDACQGDSGGPLACEKNGVAYLYGIISWGDGCGRLHKPGVYTRVANYVDWINDRIRPPRRLVAPS

SEQ ID NO: 39 - DNA, 12086 bp, Homo sapiens
  misc_feature (1)..(12086): Homo sapiens bone morphogenetic protein receptor, type II (serine/threonine kinase) (BMPR2), mRNA
gactcccccctttgtgtctggtctgctcggagccactggaagtgcctcccggagggacgcagggtgtctcgccgcctccctgcccacccccttccccggctaccttcatccgccctcccgccgccccccgccctcggtccgcgacgcccgagttccgtcaggagcccagagctgcgggagaacgaggcggcggcggcggcggcggcggcggcggcggcggcagcagcagcggcttcctcggggggttgtgattcgctcacaggagccattgacgggagaagaggaggctttcttggtggaatttacctcaggcaagatcgagccgcaggaataaaaagcgaggaagggaagggagcgccgccgggaggactagaaggggcagcctctcacacccactccgcctgccgtctcggggagcccggaccggggccgcgaccgcgacccctcccctcccccgctcctacctctcctcagccttcgccagggcctccccaaccctctcacggttgttctgcgaaggcgtggggactgtgagcttgtccatggaggcaggcaccttttttgatccagtcaaggaagaggatttgttgttttcgaaatcagagtgaaggaagcaccgaagcgaaacttaaggaatcctgccttcccggagccgcgggcgatgcgactagggctgccgggcgccgccgccgcccgtccggcttcgtccttcccggcagtcgggaactagttctgaccctcgccccccgaccccggatcgaatccccgccctccgcaccctggatatgttttctcccagacctggatatttttttgatatcgtgaaactacgagggaaataatttgggggatttcttcttggctccctgctttccccacagacatgccttccgtttggagggccgcggcaccccgtccgaggcgaaggaacccccccagccgcgagggagagaaatgaagggaatttctgcagcggcatgaaagctctgcagctaggtcctctcatcagccatttgtcctttcaaactgtattgtgatacgggcaggatcagtccacgggagagaagacgagcctcccggctgtttctccgccggtctacttcccatatttcttttctttgccctcctgattcttggctggcccagggatgacttcctcgctgcagcggccctggcgggtgccctggctaccatggaccatcctgctggtcagcactgcggctgcttcgcagaatcaagaacggctatgtgcgtttaaagatccgtatcagcaagaccttgggataggtgagagtagaatctctcatgaaaatgggacaatattatgctcgaaaggtagcacctgctatggcctttgggagaaatcaaaaggggacataaatcttgtaaaacaaggatgttggtctcacattggagatccccaagagtgtcactatgaagaatgtgtagtaactaccactcctccctcaattcagaatggaacataccgtttctgctgttgtagcacagatttatgtaatgtcaactttactgagaattttccacctcctgacacaacaccactcagtccacctcattcatttaaccgagatgagacaataatcattgctttggcatcagtctctgtattagctgttttgatagttgccttatgctttggatacagaatgttgacaggagaccgtaaacaaggtcttcacagtatgaacatgatggaggcagcagcatccgaaccctctcttgatctagataatctgaaactgttggagctgattggccgaggtcgatatggagcagtatataaaggctccttggatgagcgtccagttgctgtaaaagtgttttcctttgcaaaccgtcagaattttatcaacgaaaagaacatttacagagtgcctttgatggaacatgacaacattgcccgctttatagttggagatgagagagtcactgcagatggacgcatggaatatttgcttgtgatggagtactatcccaatggatctttatgcaagtatttaagtctccacacaagtgactgggtaagctcttgccgtcttgctcattctgttactagaggactggcttatcttcacacagaattaccacgaggagatcattataaacctgcaatttcccatcgagatttaaacagcagaaatgtcctagtgaaaaatgatggaacctgtgttattagtgactttggactgtccatgaggctgactggaaatagactggtgcgcccaggggaggaagataatgcagccataagcgaggttggcactatcagatatatggcaccagaagtgctagaaggagctgtgaacttgagggactgtgaatcagctttgaaacaagtagacatgtatgctcttggactaatctattgggagatatttatgagatgtacagacctcttcccaggggaatccgtaccagagtaccagatggcttttcagacagaggttggaaaccatcccacttttgaggatatgcaggttctcgtgtctagggaaaaacagagacccaagttcccagaagcctggaaagaaaatagcctggcagtgaggtcactcaaggagacaatcgaagactgttgggaccaggatgcagaggctcggcttactgcacagtgtgctgaggaaaggatggctgaacttatgatgatttgggaaagaaacaaatctgtgagcccaacagtcaatccaatgtctactgctatgcagaatgaacgcaacctgtcacataataggcgtgtgccaaaaattggtccttatccagattattcttcctcctcatacattgaagactctatccatcatactgacagcatcgtgaagaatatttcctctgagcattctatgtccagcacacctttgactataggggaaaaaaaccgaaattcaattaactatgaacgacagcaagcacaagctcgaatccccagccctgaaacaagtgtcaccagcctctccaccaacacaacaaccacaaacaccacaggactcacgccaagtactggcatgactactatatctgagatgccatacccagatgaaacaaatctgcataccacaaatgttgcacagtcaattgggccaacccctgtctgcttacagctgacagaagaagacttggaaaccaacaagctagacccaaaagaagttgataagaacctcaaggaaagctctgatgagaatctcatggagcactctcttaaacagttcagtggcccagacccactgagcagtactagttctagcttgctttacccactcataaaacttgcagtagaagcaactggacagcaggacttcacacagactgcaaatggccaagcatgtttgattcctgatgttctgcctactcagatctatcctctccccaagcagcagaaccttcccaagagacctactagtttgcctttgaacaccaaaaattcaacaaaagagccccggctaaaatttggcagcaagcacaaatcaaacttgaaacaagtcgaaactggagttgccaagatgaatacaatcaatgcagcagaacctcatgtggtgacagtcaccatgaatggtgtggcaggtagaaaccacagtgttaactcccatgctgccacaacccaatatgccaatgggacagtactatctggccaaacaaccaacatagtgacacatagggcccaagaaatgttgcagaatcagtttattggtgaggacacccggctgaatattaattccagtcctgatgagcatgagcctttactgagacgagagcaacaagctggccatgatgaaggtgttctggatcgtcttgtggacaggagggaacggccactagaaggtggccgaactaattccaataacaacaacagcaatccatgttcagaacaagatgttcttgcacagggtgttccaagcacagcagcagatcctgggccatcaaagcccagaagagcacagaggcctaattctctggatctttcagccacaaatgtcctggatggcagcagtatacagataggtgagtcaacacaagatggcaaatcaggatcaggtgaaaagatcaagaaacgtgtgaaaactccctattctcttaagcggtggcgcccctccacctgggtcatctccactgaatcgctggactgtgaagtcaacaataatggcagtaacagggcagttcattccaaatccagcactgctgtttaccttgcagaaggaggcactgctacaaccatggtgtctaaagatataggaatgaactgtctgtgaaatgttttcaagcctatggagtgaaattattttttgcatcatttaaacatgcagaagatgtttaaaaataaaaaaaaaactgctttatcctcctgtcagcaccccctcccacccctgcaacaaagacttgctttaaatagatttcagctatgcagaaaaatttagcttatgcttccatatttttaaattttgttttttaagttttgcacttttgtttagtctcgctaaagttatatttgtctgttatgaccacagagttatatgtgtgtgtatcaaaagtggtctcaaaatatttttttaagaaaaaaagcaaaaacaatgtattgctgataatcagtttggaccagtttcttaaggtcattaaaacagaagcaaattaagacaggtttgactgcagtggtgtctggtatccatgttttatttctgggcacaagctagtttttatgttgatacgttcctgaacatattatcttgttggacatcttttctcttgtgttttgtttgaatgtgcaatagtttataggccacaaataagctttcttgtaagctctcttcctaacagggcacatattcttccataatataaacacttttctgccccatctcccatacttttgaaggtcagttctatgacagtgaattttgcacaggagaagcagctacctgatttcttactttctctctccttatcatggagaatacagaaacattgtctgaaagggctctaaagaaggaactaccaaaacctgacttgaaatgccatttcttttaaccttccaaatcctaaatgtttccttcaaggcatcttaataaacttatttgcttctggttttgggagttcataagagagaatagaacaaaatacaggacatcaaatattagccatttcccattttattttatttttctatgtaggttcatgttccatgttcatttatttaagaaatacatttttattggtaagcttatagagctacacttatggaatttttaagtaggtaaataaatggttaagacaaaatagtgttatagccttcattctctgaataggccatctttgactcataaaattacccttactgtttattataacttcagaagtaatttatagttctgaacctatagtatcttttaccctgttcccaagcaaagactggtgactttatctgaaaatgattcctcttcccatgacctaaaacactgtgaggaaaaatcattcaagtggcatgccaagtccctatgaaggaagggctgctatcaaacctaccttttttgagcaaactgagactaaacttctctcttttcaaaattgtgttatcttccttaatcctattttcataatttttccttttgccagtttttcacattatctttgatatgtgagcaacatttattatttacattagagtataccttttagtaataaaatgacttgaaatcatattatttttaaaagccctttgcttctttcattacttataatctcctctaaaacaacctctgcatgttttttttaaataaagcactttctgtcaaataatggacttttttctaaacagaaattattttctattaatttgcaaactgatgatttcacttttttttacttttttttcgattatcagagtacttagtaaatgttatatagtttagttctaagatagttccagggattaaaaggttaagaggaaaacacaaatcaccaaatttctgatttatgtttttatctcctgaacaatattttctcactcatattcctctatcttatcacttagctaaagacagcctaaatttcccaattttctgtccaaaatatttgtgatttacttgtatataagccttctcatttgccatgtgctgtgatcttacaagttagatgttactatacctaccatttattcagctggattgctgaacacagttctggattcatagaattaagaatatcttgttagtgcccaggatttccacgttttgtgttttattggcccttttctttattcagccccttaatctattttcagtcttctggcacgtaatttttttcacagttattattcttctattaacaaattatttttatctttcctagtacattttcacttagttctcttgcccttaaatatctttcacatgcattttaggatattcttttcaaatatttgtaggacaactttgaatcaaaataaattatgttccttctccaatttgaagcattgaggataaatgaccatttgaggtctaactgatcttttcctgccagaagagttatcttacgttctgctatatttgtatttgggccagttgattgtaggttgtccaacattttttaatattgggaaaattatgataaaatgctttaaaaattaatatgccagattaaaataactgaatagtttactatttcattcaagcatgtttaaaacaaataatttcctttcaccagtttttcttagtaaactcctgaaaaagtaggaaaggtggaaagtatatatcatttttataaattttaaattgtacatcagacttttaaaatctgtaatatacaagcaagcaaaattattttaaatgacttaattgtatgctaatactcatctgataataaatgcttcttaaagttgacatttaactgctatcacaaagttttatatgtagaaaagtggggtccttttgaataaaagatcattcaactaaaaatattaaaatttatttcactggatggtaatgtaaccttaaaagcatcataataggtaaagtctaatattagttcccttaacaaaatcctaactgtataccagaattaggtcactgaaagaacttgatttgaattacgtttagacaaaaatgatttaattgtaaattcttaaaactttctaaatgcataattggcaaaaaaaaaaacccactgttaccagtgtaggaagttacaagaaggcacatactgaatgctgaagtatacatatgctatttctcttaaacctcagagcaaccatatgagcattgtaattaatattcccattttacagatgaggaaactgaagctaagagaagctaagtaatatgcccaaggtccacatctagtaacagacaaagctgggatttcagtctatgtctgcctctctccacatctctttcatccataccacactgcctacatgccatatgacaggatgtgtaatgggctaacgtttatttaaaaagttcaggccaggtgcagtggcttatgcctataatccccgcactttgggaggccgagccgggtggatcatgaggtcagacgttcaacaccagcctggccaagatggtgaaaccccgtctctattaaaaatattttttaaaaattaactgagcgtggtggtgggcgcctgtagtcccagctactcaggaggctaaggcaggagaatctcttgaacccgagaggtggaggttgcagtgagctgagatcgcgccactgcactgcagcctaggcgacagagcaagactgcctcaaaaaaaaaacataaaaattcagtcactatactctggcacaattttcatttgtatatgagccaaaccacataccttaatgatttgggaagttaggcaaatatgagatatgtagacatatctatgctgattgttgcttgagaaataattactaattctagacaaaactcaatcctgtatcttccatcatgaatcttaaaatcatttcacttcactcctaacatttcttacattgcagaactaatggtaaagtaaattttacggaaaaagttaagatagctttgggaacagagacctttccctaaattgattccatagcagatttgggggaattaacaaagaatttcagtctcatcaatcctttgaatccatcttcaaaacttctgcttttaataactttagaaaatttactaatctatagaactaattgagtaggatataggaaggatacaaggatataatgtcctttttataaaagtttagtatagcttctttacatgtatccacttgttccagaaaatgtgcattggttctgaatgtgaaaatatttaaagagagaaaggaacactcaagtaagtgtgggcttcagtgggaattatcacaaaacattggcaagtatttttatttaaattattttcaaatttgacttctacagccaagtggaattggtaggctgtagctgttacactgaaatttctagtctttgtaagtgcctcctgaaagtcatttaaaatggaaaaatatttcaatgagcttttccttttttcatatttatggacatgaatattttattggagatcattaactcctagaatttgagattatatttccatacaacattttataaagttatgttgaacttactacctgttatgtgcaggttattatgtaactattcacagattgcttcatatattgctttatcttcccatctaacttcttaaagttaaaatccggacacacatgttgattatctagaccagtcattctggaaattgtaacactcccacataaaccccaggagactttttcagaatgcaatgtttctaaatgtactgttactggcagtttactctccagcatataaggttgcattttaacttttagattatgaactgtgcaaactttacccaaaactatcttgcatgattccctcctaaatatattccttgattaagtaaatctggcaaatcactgtttgagctagttacataaaatttgttatcaagagaaggcttttctacaagtttccagattaacataaagaaaagagggaatcacagggcatttaagtgcaccttcccattactttccttaaatcacctcatagttaggctgggcgcggtggctcacgcctgtaatcccagcactttgggaggccgagacggtggatcacgaggtcaggggactgagatcatcctggctaacacgatgaaacctcatctctactaaaaatacaaataattagccaggcatggtggcacgcgcctgtagtcccagctactcgggaggcagagacaggagaatcgtttgcacccgggaggcggaggttgttgcagtgagccgagatcgcgccaatgcactccagcctgggctacagagtgagactccatctcaaaaaaaaacaaaaaaaatcacctcatagtttatgtctgacttactccaaacctcagttatctgatttgtagtctgtgtcaggaaagttttcttcatatctattctgtctccctcctctttgattttaaatttttttcttttacccagtaggacaaaaaagagcagttggtcatcatccccaatattcttagtcttcagtatgcttcaggcctctcaatgaacacttaagtctcaattcttcagacaaaattgcttaagctcttctcctcagtcctattttaatgactttatatttaagaatatagaattatattttcttttatattcaaattcattactccagttaagtaatagtttgatagtttgatagaatcgagagttaagatgtttctatttgaaagtggattcaaccatcagaccaccagcaaatcggcacttaatttttgtgttatctaacattttctattgtggaattttatgattttatattctcattagttataactaaaaagccatgcacacagaattgtatatcattttgccattaaaattttttaacatattgcagcaagcttagttttatgattgagccacaaccttttacatattttttgtatgaaatattaaacactaaatgcaagattaactttcaaaagcaaaccctacattaatcaggtattatctatggacatttttgtagaccacttttgaaatacttattattttgcaacatagactggactatacaactttcatttaacttttaggtgactgatttaagttgagtgtgcatatagagaaaaacctagaaatttatctcatggcagatacatttgaaagtacttcagaagaatttatgctgtatattaaaactaggctcaaaataaatctatcgtatctttaaaagtccaattctgttattactgtgatgtttgtagtgttactattaaacattgtgaacatacacatttttaaaacaacttgaaacccattttaaaatctgggtaagagagaaggaatcttcagaacaaaatcacatcattagggtgtccagtttatgattgaatttttaagcaaattactgtatttgaaactacaacttgatttggttttcagttttaaaaggcaacatgtgggttttatccattttatttatacctttagatttcagaaacatcttcatgttttagatgcattctacagacatcatgttacttaaaaactcagggcccctttcatccctttgtacactgaaaaagttcaattgttagcaagtaagcaattagatccagttgaatatttaaagtgtttgttgcacagttcatttaatgtttcatcttatttgactttttcacatagatataatatcagatttcattaattataaaaagttgcccagttctgtaattactgaacagagggaatgactcaactaattggctacatgttgcaacaaatttaggcctttagagttgaagcactgacttaaaacgacttacatttctgttctttggtcaaatgaccatacatgatatgggacaaattgtttcattttgtttgttttttaataagggaacttggtaaagtagttcctgtcagataggattttctcaagagacaatttaacgttataaagccttctaaaagtgaactaaatattttataactttagtaatagcttggatggttttgagaaaataacctgtatttatcacattgtcaaacagaatttttctttgaatcagacaagttcaagctctaaattgatgtgctatatacttaaaatcctaggaagttatctgtaaccagtctcttgtctcaggctcttcaccttgttaccaatcctcgtaagtatgtaaaggaaacatatttttaaagaagcttaacagtaagaaaaaattactaaaagatgcaattcaaagataggtcccagtttaacactgaattgcttgacttctgtggcttttctttttctggccacatttatttatttaagcaatttttgtatgccttgttatttcatttccatagagattatattgtatcagtgtttatgtaagctggaatcatcctcagttttttgctgataatttttcaaataaagatacatggataattgtaaaatacactaactcttagggtgttgtagtagctgaaacatggagatgcgtagctgtcatgctttttctgaatggacaggagaaacataagctacggagtattcacttctgaggatgcttttccggaaaaagaaaggctagaaaatactcgcacttcctcagaaccctctttcttgttaacgggtatcttttgttggtgtgttttgctcttacattacagatagactatcatatatgactttatgaataatttcagttattttgcttttgtataagctgtctgaagccttgctatgctgtataagttgtgtttgatggatcagtgtgagtataaaataaagcaaatcacttttcttttgtattatctatggatgccactatgaaagctgacattaagccactaaagagttttctatgaataagtgtaagtaaatgctttgatatatataaacctaaataaaaagattgtattgatacagagacattggagaaggagattttaaggcagttctttaggtttaaaaaggcttgctgtaaaatggtgcgttattccgtttattaaagatcatattaatgacaataaaaaaaaaaaaaaaaa

SEQ ID NO: 40 - protein, 1038 aa, Homo sapiens
  misc_feature (1)..(1038): bone morphogenetic protein receptor type-2 precursor
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 41 - DNA, 4260 bp, Homo sapiens
  misc_feature (1)..(4260): Homo sapiens growth differentiation factor 11 (GDF11), mRNA
tccccgccccccagtcctccctcccctcccctccagcatggtgctcgcggccccgctgctgctgggcttcctgctcctcgccctggagctgcggccccggggggaggcggccgagggccccgcggcggcggcggcggcggcggcggcggcggcagcggcgggggtcgggggggagcgctccagccggccagccccgtccgtggcgcccgagccggacggctgccccgtgtgcgtttggcggcagcacagccgcgagctgcgcctagagagcatcaagtcgcagatcttgagcaaactgcggctcaaggaggcgcccaacatcagccgcgaggtggtgaagcagctgctgcccaaggcgccgccgctgcagcagatcctggacctacacgacttccagggcgacgcgctgcagcccgaggacttcctggaggaggacgagtaccacgccaccaccgagaccgtcattagcatggcccaggagacggacccagcagtacagacagatggcagccctctctgctgccattttcacttcagccccaaggtgatgttcacaaaggtactgaaggcccagctgtgggtgtacctacggcctgtaccccgcccagccacagtctacctgcagatcttgcgactaaaacccctaactggggaagggaccgcagggggagggggcggaggccggcgtcacatccgtatccgctcactgaagattgagctgcactcacgctcaggccattggcagagcatcgacttcaagcaagtgctacacagctggttccgccagccacagagcaactggggcatcgagatcaacgcctttgatcccagtggcacagacctggctgtcacctccctggggccgggagccgaggggctgcatccattcatggagcttcgagtcctagagaacacaaaacgttcccggcggaacctgggtctggactgcgacgagcactcaagcgagtcccgctgctgccgatatcccctcacagtggactttgaggctttcggctgggactggatcatcgcacctaagcgctacaaggccaactactgctccggccagtgcgagtacatgttcatgcaaaaatatccgcatacccatttggtgcagcaggccaatccaagaggctctgctgggccctgttgtacccccaccaagatgtccccaatcaacatgctctacttcaatgacaagcagcagattatctacggcaagatccctggcatggtggtggatcgctgtggctgctcttaaggtgggggatagaggatgcctcccccacagaccctaccccaagacccctagccctgcccccatccccccaagccctagagctccctccactcttcccgcgaacatcacaccgttccccgaccaagccgtgtgcaatacaacagagggaggcaggtgggaattgagggtgaggggtttgggggaaaggggaagcaggggcatagtcagggtggggagtgtttgaagtttgcagatgagaaggtttgacaaaaagacagagagatgtagagacagtgatagagacagaggaacaaaaagagcagcagtgagaaggcaaagagagaggcagaagagacagacgaggcagagacaaaacactgagaaagagactgaaatggagtaataaatgaaagccccacaccaagcctcctttcttccactggcaaggtgaggggcttggtatagtttggggagatcccctgactattcagtaggagaagaaatcaaaaatccattcttttctccttctctccctccaacagtggccaggggaaggggaagtgagggcaggggcaaaaagatttgggaatttttatttatttatttattgtgacttttcatttttttggtatttggctttactggaataggagggcccctgcccactgtgccccgtttatcccttattccccaaaccctgctctccccaacacctactcacttaagcacttgtataaagcctccagggttgggaatgggagtaaagggcaagagggcggacacatgaagtttagtttctaacccatcatcaccctaactcaaccttttctgagccaaatggcttgaattgaagccagttgtcatggaaatagtaagaggttagggtttaagagctggggatgcgggggtgggagagagaaccctcaacatccaggatctatataatgagagctactttaaaccctcaggtccaccctcatgatgctgagttatttagccagagggtgcagcctgcttatgcccaaattccctcagccaagagagagaccaaagagcctctggaatggccctgctcccagcctctatcttcaggtcaattagagagagtatagagaccccagagtcccctgggtctggaaagcgttaggagaggtcaagaaaggagcagtaaggaggctgaaggttacagggcatttgaatccaaatcactgctctgggctagggaatagagccagcagaccaaggtgggaaggattctggaagggggacattttagtctcctaaccccaaagctcagggtggaagaggggagaacaaggaagcagagtgtataattattttttccttttatttttggaatctaacagtacctggcagcagggaggggaaagtacagtggggaaaagcatctgacaaggccagttagaacagaggatgggaaggatggagactcccgggcttggaaggctaggaagcaggcagagactggttgccatttcaagtcactagctaggcccattcattcctcccacaaccctgacccattctcctctggactcactgtgcctcagtttcttcccctcaatggaatgagaaatgacagcacccgccacagccaagagatgaattctgagcacttaccacgggcactttatggacataaaatacctctcgctgtgggacagataaccagggcaccagagtagtggtgaagagatgtgaggcttaagaggagtcacaggcttcagagtacaagttcccctctgcctcccagctggacagtgcctagaagccaaggagttgagaatctcctgatccacaccctatccttacttcaccaccaggcctcttggctccaggcaagagcttagaggatgtcaggagaggtgggggtaagaatcttcagcaaaactgtcactctaagtagagccagcagttacgggtctgataaaaacagtactgaactaaagtaaagcccaagctggtgagcaaaactggatggctcattcttcccaagagcatgactctcccccttggccagttggtggaaggggcaaaggtatgtgaccacccttgagaaggtgatgttggtgagctttaacatcttattcctattcttatagtgagaaagtgaaacaagatctttcagtagaggaatgggcagggctgttaggctcttcagcttgccttcacccatatagcagctatgctaaccccaagcctctctggccctgttcttcatccttccttctgccccaatcctgaaggacaagacacacccggccatcaacaccactcacatttccttggtggaaggaaaggaacagagaagtgaagaacagatacctccctccaaggtcaaatgcctcgtgatcttggcagagtagggattgggcaataagcatcaggtatcttccctctacagattctagagagctggggcattaaatatgggggacacttagaatacagctccttaaataccaccaaataaagacctttgtgtgtgtgtggtgggtggggggggggcaggggtctttctcttatgaacataaatctgtgagctgaagtctcattcccctgttcctccctacccccaaagaggcacagagtgaagggacttggggggcacagctcagcaacccagtgggagttagcaccccctcccaccttatgatgtgtgtggacctggccagtgcccctctgaacatatcattattagtgtaattatcatttattttgtgtatttgtcacattgtgtgcatgacagcctttgttaagggtgtctgaggagtatggagctgacaggggcattggaatgccaggaaagaacttcttcaactgagatcaaggcttcctggagggaaccactgcaaaaaggccatcaggcagttttcaagttatgtgacagagggcaaagacggccatagggtgctctgagttttgggatggtcacatgacacaatccagcacttgaacctgaaaaaaaaaataaaagcggtcaaagagtttagaattca

SEQ ID NO: 42 - protein, 407 aa, Homo sapiens
  misc_feature (1)..(407): growth/differentiation factor 11 precursor
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 43 - DNA, 1051 bp, Homo sapiens
  misc_feature (1)..(1051): Homo sapiens insulin-like growth factor binding protein 7 (IGFBP7), transcript variant 2, mRNA
actcgcgcccttgccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtacacaataaatctcacagccatttaaaaatgactagtacatttgctttaaaaagaacagaactaagtatgaaagtatcagacgtagctattgatgaaattctgtagttagcaacccataagggcattaagtatgccattaaaatgtacagcatgagactccaaaagattatctggatgggtgactg

SEQ ID NO: 44 - protein, 279 aa, Homo sapiens
  misc_feature (1)..(279): insulin-like growth factor-binding protein 7 isoform 2 precursor
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGTQ

SEQ ID NO: 45 - DNA, 980 bp, Homo sapiens
  misc_feature (1)..(980): Homo sapiens insulin-like growth factor binding protein 6 (IGFBP6), mRNA
gcggcggcgggcagcagctgcgctgcgactgctctggaaggagaggacggggcacaaaccctgaccatgaccccccacaggctgctgccaccgctgctgctgctgctagctctgctgctcgctgccagcccaggaggcgccttggcgcggtgcccaggctgcgggcaaggggtgcaggcgggttgtccagggggctgcgtggaggaggaggatggggggtcgccagccgagggctgcgcggaagctgagggctgtctcaggagggaggggcaggagtgcggggtctacacccctaactgcgccccaggactgcagtgccatccgcccaaggacgacgaggcgcctttgcgggcgctgctgctcggccgaggccgctgccttccggcccgcgcgcctgctgttgcagaggagaatcctaaggagagtaaaccccaagcaggcactgcccgcccacaggatgtgaaccgcagagaccaacagaggaatccaggcacctctaccacgccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaaagctgggggatagaggggctgcagggccactggaaggaacatggagctgtcatcactcaacaaaaaaccgaggccctcaatccaccttcaggccccgccccatgggcccctcaccgctggttggaaagagtgttggtgttggctggggtgtcaataaagctgtgcttggggtcgctgaaaaaaaaaaaaaa

SEQ ID NO: 46 - protein, 240 aa, Homo sapiens
  misc_feature (1)..(240): insulin-like growth factor-binding protein 6 precursor
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 47 - DNA, 1234 bp, Homo sapiens
  misc_feature (1)..(1234): Homo sapiens apolipoprotein E (APOE), transcript variant 2, mRNA
gggacagggggagccctataattggacaagtctgggatccttgagtcctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 48 - protein, 317 aa, Homo sapiens [sequence omitted]

SEQ ID NO: 49 - DNA, 1789 bp, Homo sapiens
  misc_feature (1)..(1789): Homo sapiens phospholipase A2, group VII (platelet-activating factor acetylhydrolase, plasma) (PLA2G7), transcript variant 2, mRNA
ggaaaaccggcctgactggggggtgaattcagcagggagtaaatctgatcggcatcaggtctgcggaaaggagctggtgagcacgacaccacccaggcattgcctggctctctccgcggcgggctaagttaaccgcgggtccaggagactaagctgaaactgctgctcagctcccaagatggtgccacccaaattgcatgtgcttttctgcctctgcggctgcctggctgtggtttatccttttgactggcaatacataaatcctgttgcccatatgaaatcatcagcatgggtcaacaaaatacaagtactgatggctgctgcaagctttggccaaactaaaatcccccggggaaatgggccttattccgttggttgtacagacttaatgtttgatcacactaataagggcaccttcttgcgtttatattatccatcccaagataatgatcgccttgacaccctttggatcccaaataaagaatatttttggggtcttagcaaatttcttggaacacactggcttatgggcaacattttgaggttactctttggttcaatgacaactcctgcaaactggaattcccctctgaggcctggtgaaaaatatccacttgttgttttttctcatggtcttggggcattcaggacactttattctgctattggcattgacctggcatctcatgggtttatagttgctgctgtagaacacagagatagatctgcatctgcaacttactatttcaaggaccaatctgctgcagaaataggggacaagtcttggctctaccttagaaccctgaaacaagaggaggagacacatatacgaaatgagcaggtacggcaaagagcaaaagaatgttcccaagctctcagtctgattcttgacattgatcatggaaagccagtgaagaatgcattagatttaaagtttgatatggaacaactgaaggactctattgatagggaaaaaatagcagtaattggacattcttttggtggagcaacggttattcagactcttagtgaagatcagagattcagatgtggtattgccctggatgcatggatgtttccactgggtgatgaagtatattccagaattcctcagcccctcttttttatcaactctgaatatttccaatatcctgctaatatcataaaaatgaaaaaatgctactcacctgataaagaaagaaagatgattacaatcaggggttcagtccaccagaattttgctgacttcacttttgcaactggcaaaataattggacacatgctcaaattaaagggagacatagattcaaatgtagctattgatcttagcaacaaagcttcattagcattcttacaaaagcatttaggacttcataaagattttgatcagtgggactgcttgattgaaggagatgatgagaatcttattccagggaccaacattaacacaaccaatcaacacatcatgttacagaactcttcaggaatagagaaatacaattaggattaaaataggttttttaaaagtcttgtttcaaaactgtctaaaattatgtgtgtgtgtgtgtgtgtgtgtgtgtgtgtgagagagagagagagagagagagagagagagagagagaattttaatgtattttcccaaaggactcatattttaaaatgtaggctatactgtaatcgtgattgaagcttggactaagaattttttccctttagatgtaaagaaagaatacagtatacaatattcaaaaaaaaaaaaaaaaaaaaaaaaaaaaaaaaaaaaaaaaaa

SEQ ID NO: 50 - protein, 441 aa, Homo sapiens
  misc_feature (1)..(441): platelet-activating factor acetylhydrolase precursor
MVPPKLHVLFCLCGCLAVVYPFDWQYINPVAHMKSSAWVNKIQVLMAAASFGQTKIPRGNGPYSVGCTDLMFDHTNKGTFLRLYYPSQDNDRLDTLWIPNKEYFWGLSKFLGTHWLMGNILRLLFGSMTTPANWNSPLRPGEKYPLVVFSHGLGAFRTLYSAIGIDLASHGFIVAAVEHRDRSASATYYFKDQSAAEIGDKSWLYLRTLKQEEETHIRNEQVRQRAKECSQALSLILDIDHGKPVKNALDLKFDMEQLKDSIDREKIAVIGHSFGGATVIQTLSEDQRFRCGIALDAWMFPLGDEVYSRIPQPLFFINSEYFQYPANIIKMKKCYSPDKERKMITIRGSVHQNFADFTFATGKIIGHMLKLKGDIDSNVAIDLSNKASLAFLQKHLGLHKDFDQWDCLIEGDDENLIPGTNINTTNQHIMLQNSSGIEKYN

SEQ ID NO: 51 - DNA, 2121 bp, Homo sapiens
  misc_feature (1)..(2121): Homo sapiens cyclin-dependent kinase 2 (CDK2), transcript variant 3, mRNA
aacgcgggaagcaggggcggggcctctggtggcggtcgggaactcggtgggaggcggcaacattgtttcaagttggccaaattgacaagagcgagaggtatactgcgttccatcccgacccggggccacggtactgggccctgtttccccctcctcggcccccgagagccagggtccgccttctgcagggttcccaggcccccgctccagggccgggctgacccgactcgctggcgcttcatggagaacttccaaaaggtggaaaagatcggagagggcacgtacggagttgtgtacaaagccagaaacaagttgacgggagaggtggtggcgcttaagaaaatccgcctggacacgctgctggatgtcattcacacagaaaataaactctacctggtttttgaatttctgcaccaagatctcaagaaattcatggatgcctctgctctcactggcattcctcttcccctcatcaagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcgcaaatgctgcactacgaccctaacaagcggatttcggccaaggcagccctggctcaccctttcttccaggatgtgaccaagccagtaccccatcttcgactctgatagccttcttgaagcccccagccctaatctcaccctctcctccagtgtgggcttgaccaggcttggccttgggctatttggactcaggtgggccctctgaacttgccttaaacactcaccttctagtcttggccagccaactctgggaatacaggggtgaaaggggggaaccagtgaaaatgaaaggaagtttcagtattagatgcacttaagttagcctccaccaccctttcccccttctcttagttattgctgaagagggttggtataaaaataattttaaaaaagccttcctacacgttagatttgccgtaccaatctctgaatgccccataattattatttccagtgtttgggatgaccaggatcccaagcctcctgctgccacaatgtttataaaggccaaatgatagcgggggctaagttggtgcttttgagaaccaagtaaaacaaaaccactgggaggagtctattttaaagaattcggttgaaaaaatagatccaatcagtttataccctagttagtgttttgcctcacctaataggctgggagactgaagactcagcccgggtggggctgcagaaaaatgattggccccagtccccttgtttgtcccttctacaggcatgaggaatctgggaggccctgagacagggattgtgcttcattccaatctattgcttcaccatggccttatgaggcaggtgagagatgtttgaatttttctcttccttttagtattcttagttgttcagttgccaaggatccctgatcccattttcctctgacgtccacctcctaccccataggagttagaagttagggtttaggcatcattttgagaatgctgacactttttcagggctgtgattgagtgagggcatgggtaaaaatatttctttaaaagaaggatgaacaattatatttatatttcaggttatatccaatagtagagttggctttttttttttttttttggtcatagtgggtggatttgttgccatgtgcaccttggggttttgtaatgacagtgctaaaaaaaaaaagcattttttttttatgatttgtctctgtcacccttgtccttgagtgctcttgctattaacgttatttgtaatttagtttgtagctcattaaaaaaatgtgcctagttttatagttca

SEQ ID NO: 52 - protein, 238 aa, Homo sapiens
  misc_feature (1)..(238): cyclin-dependent kinase 2 isoform 3
MENFQKVEKIGEGTYGVVYKARNKLTGEVVALKKIRLDTLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 53 - DNA, 2811 bp, Homo sapiens
  misc_feature (1)..(2811): Homo sapiens cyclin A2 (CCNA2), mRNA
ccatttcaatagtcgcgggatacttgaactgcaagaacagccgccgctccggcgggctgctcgctgcatctctgggcgtctttggctcgccacgctgggcagtgcctgcctgcgcctttcgcaacctcctcggccctgcgtggtctcgagctgggtgagcgagcgggcgggctggtaggctggcctgggctgcgaccggcggctacgactattctttggccgggtcggtgcgagtggtcggctgggcagagtgcacgctgcttggcgccgcaggctgatcccgccgtccactcccgggagcagtgatgttgggcaactctgcgccggggcctgcgacccgcgaggcgggctcggcgctgctagcattgcagcagacggcgctccaagaggaccaggagaatatcaacccggaaaaggcagcgcccgtccaacaaccgcggacccgggccgcgctggcggtactgaagtccgggaacccgcggggtctagcgcagcagcagaggccgaagacgagacgggttgcaccccttaaggatcttcctgtaaatgatgagcatgtcaccgttcctccttggaaagcaaacagtaaacagcctgcgttcaccattcatgtggatgaagcagaaaaagaagctcagaagaagccagctgaatctcaaaaaatagagcgtgaagatgccctggcttttaattcagccattagtttacctggacccagaaaaccattggtccctcttgattatccaatggatggtagttttgagtcaccacatactatggacatgtcaattgtattagaagatgaaaagccagtgagtgttaatgaagtaccagactaccatgaggatattcacacataccttagggaaatggaggttaaatgtaaacctaaagtgggttacatgaagaaacagccagacatcactaacagtatgagagctatcctcgtggactggttagttgaagtaggagaagaatataaactacagaatgagaccctgcatttggctgtgaactacattgataggttcctgtcttccatgtcagtgctgagaggaaaacttcagcttgtgggcactgctgctatgctgttagcctcaaagtttgaagaaatataccccccagaagtagcagagtttgtgtacattacagatgatacctacaccaagaaacaagttctgagaatggagcatctagttttgaaagtccttacttttgacttagctgctccaacagtaaatcagtttcttacccaatactttctgcatcagcagcctgcaaactgcaaagttgaaagtttagcaatgtttttgggagaattaagtttgatagatgctgacccatacctcaagtatttgccatcagttattgctggagctgcctttcatttagcactctacacagtcacgggacaaagctggcctgaatcattaatacgaaagactggatataccctggaaagtcttaagccttgtctcatggaccttcaccagacctacctcaaagcaccacagcatgcacaacagtcaataagagaaaagtacaaaaattcaaagtatcatggtgtttctctcctcaacccaccagagacactaaatctgtaacaatgaaagactgcctttgttttctaagatgtaaatcactcaaagtatatggtgtacagtttttaacttaggttttaattttacaatcatttctgaatacagaagttgtggccaagtacaaattatggtatctattactttttaaatggttttaatttgtatatcttttgtatatgtatctgtcttagatatttggctaattttaagtggttttgttaaagtattaatgatgccagctgtcaggataataaattgatttggaaaactttgcaagtcaaatttaacttcttcaggattttgcttagtaaagaagtttacttggtttactatataatgggaagtgaaaagccttcctctaaaattaaagtaggtttaggaaaacagaccctcaaattctgacattcattttcctaagcaactggatcaatttgctgacttgggcataatctaatctaagcatatctgaatacagtattcagagatagatacagtagagattccccagactttttcgctctttgtaaaacctgtttgtttaggttttgcgaggtaaactcaacagaggttgggagtggaagagggtgggaagcttatatgcaaattaacagacgagaaatgctccagaaggtttattattttaaagcacattaaaaacaaaaaactatttttaaaatcctgctagattttataatggatttgtgaataaaaaatacccagggttctcagaatggaataaatatcccttttaatagttatatatacagatatacaactgttagctttaattggcagctctcttcttttttcttcttttcactggctttttacttggtgctttttcttgttttgcactggtggtctgtgttctgtgaataaagcaaagtaagaatttactaagagtatgttaagttttggattattgaaataagaggcatttcttagttttccagtaggatctaaaatgtgtcagctatgagtaagactggcatccaagaagtttatattatagatttaggtcctaatttttataaatcacaaggtaaaaaaatcacagaacagatggatctctaatgaaaaagggatgtctttttgtttatagtcatgtggcaagatgagagtaaaaccagagagcaaacctctataagtgttgagtatatgtatacatttgaaataaaccagaaatttgttaccttaaaaaaaaaaaaaa

SEQ ID NO: 54 - protein, 432 aa, Homo sapiens
  misc_feature (1)..(432): cyclin-A2
MLGNSAPGPATREAGSALLALQQTALQEDQENINPEKAAPVQQPRTRAALAVLKSGNPRGLAQQQRPKTRRVAPLKDLPVNDEHVTVPPWKANSKQPAFTIHVDEAEKEAQKKPAESQKIEREDALAFNSAISLPGPRKPLVPLDYPMDGSFESPHTMDMSIVLEDEKPVSVNEVPDYHEDIHTYLREMEVKCKPKVGYMKKQPDITNSMRAILVDWLVEVGEEYKLQNETLHLAVNYIDRFLSSMSVLRGKLQLVGTAAMLLASKFEEIYPPEVAEFVYITDDTYTKKQVLRMEHLVLKVLTFDLAAPTVNQFLTQYFLHQQPANCKVESLAMFLGELSLIDADPYLKYLPSVIAGAAFHLALYTVTGQSWPESLIRKTGYTLESLKPCLMDLHQTYLKAPQHAQQSIREKYKNSKYHGVSLLNPPETLNL

SEQ ID NO: 55 - DNA, 2568 bp, Homo sapiens
  misc_feature (1)..(2568): Homo sapiens mitogen-activated protein kinase- activated protein kinase 3 (MAPKAPK3), transcript variant 2, mRNA
acgtgggcgccggcagcgcgactctcggccctgggatttctgcggccgccagctcccgcgaccgcctctcctgcccctcgccggtacctcagcaaggtgcgttgccgccaggtgccactagaagcgccaggctggggccgcctctgagcgccccgcgggggccatggatggtgaaacagcagaggagcaggggggccctgtgcccccgccagttgcacccggcggacccggcttgggcggtgctccgggggggcggcgggagcccaagaagtacgcagtgaccgacgactaccagttgtccaagcaggtgctgggcctgggtgtgaacggcaaagtgctggagtgcttccatcggcgcactggacagaagtgtgccctgaagctcctgtatgacagccccaaggcccggcaggaggtagaccatcactggcaggcttctggcggcccccatattgtctgcatcctggatgtgtatgagaacatgcaccatggcaagcgctgtctcctcatcatcatggaatgcatggaaggtggtgagttgttcagcaggattcaggagcgtggcgaccaggctttcactgagagagaagctgcagagataatgcgggatattggcactgccatccagtttctgcacagccataacattgcccaccgagatgtcaagcctgaaaacctactctacacatctaaggagaaagacgcagtgcttaagctcaccgattttggctttgctaaggagaccacccaaaatgccctgcagacaccctgctatactccctattatgtggcccctgaggtcctgggtccagagaagtatgacaagtcatgtgacatgtggtccctgggtgtcatcatgtacatcctcctttgtggcttcccacccttctactccaacacgggccaggccatctccccggggatgaagaggaggattcgcctgggccagtacggcttccccaatcctgagtggtcagaagtctctgaggatgccaagcagctgatccgcctcctgttgaagacagaccccacagagaggctgaccatcactcagttcatgaaccacccctggatcaaccaatcgatggtagtgccacagaccccactccacacggcccgagtgctgcaggaggacaaagaccactgggacgaagtcaaggaggagatgaccagtgccttggccactatgcgggtagactacgaccaggtgaagatcaaggacctgaagacctctaacaaccggctcctcaacaagaggagaaaaaagcaggcaggcagctcctctgcctcacagggctgcaacaaccagtagctcatggggccttggaggagcctggcctctcagcctgcataacagactgaaatgtgctcaggccctggccaggagggcccagggtcattcttttaacaaaaggattattttgttgtgttttaatttgtcactcggaacttcaggatggaggaccctgaccctaaacctccttcagatctctggcccaggctcaagccctagagatgggcagggcctaggggctgggagctgcctgctgccatagcagcacctttagctaggttggcccgagtgaggcctctgtgctgtcctgccctggtgcatggccttagctttctaggccactgggagttgtggctgggcttcccatcttccacagagacatctccctgtgggatgggcagatgggcctggccttgagaaaggcattggccattggttgccatggtgaccagggaccacgttgctgcctgtgaatgctgagtgagcgagtaagggaggaggggcgattgagggttcacctctgccttggggaggctgatttctcacacactggctggccctctcattctcactcctccttgggccctgaggctgctggatccggtctgcctgcctccctgtgcagtccagccctgccttgctgcagccccagcccagatggcactcagcgctctcccctgagggagtccctgggcctagccatcccctcactattcccgacccaaagggtgacttttcatctgaacttaaggtgggagatatttttaacttttttccactttggaaaatgtcactgtgacaaaagccagcatactttccctgcacccatctgctcaccagatctcaggcaggaaagcccctctctgttgaagtcaggggctatcttttggtatacttgtgtgaaagtggctggttgggagcagagctaagtggcttcccattaacctgaggtctctttctttactctgggtcagacctgaggttggggaaggcgactgagccatgctcagaatgtctggtcctggcttgggcctgagtagggcagagagggcctttcctggctgatcagagcttaccagccccaccccaccatggtagccttagggtgctgagtgcctgatactgcctgacaagtgcctgacacgcagcctagttccttcctggcccctctctcactggctgggaaaccctagaccatgtcagataggacaacactgctgggttttacatccagatagtaataaacaccatttcatcattttctcttggaaaaa

SEQ ID NO: 56 - protein, 382 aa, Homo sapiens
  misc_feature (1)..(382): MAP kinase-activated protein kinase 3
MDGETAEEQGGPVPPPVAPGGPGLGGAPGGRREPKKYAVTDDYQLSKQVLGLGVNGKVLECFHRRTGQKCALKLLYDSPKARQEVDHHWQASGGPHIVCILDVYENMHHGKRCLLIIMECMEGGELFSRIQERGDQAFTEREAAEIMRDIGTAIQFLHSHNIAHRDVKPENLLYTSKEKDAVLKLTDFGFAKETTQNALQTPCYTPYYVAPEVLGPEKYDKSCDMWSLGVIMYILLCGFPPFYSNTGQAISPGMKRRIRLGQYGFPNPEWSEVSEDAKQLIRLLLKTDPTERLTITQFMNHPWINQSMVVPQTPLHTARVLQEDKDHWDEVKEEMTSALATMRVDYDQVKIKDLKTSNNRLLNKRRKKQAGSSSASQGCNNQ

SEQ ID NO: 57 - DNA, 1906 bp, Homo sapiens
  misc_feature (1)..(1906): Homo sapiens kallikrein-related peptidase 3 (KLK3), transcript variant 3, mRNA
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 58 - protein, 238 aa, Homo sapiens
  misc_feature (1)..(238): prostate-specific antigen isoform 3 preproprotein
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 59 - DNA, 3173 bp, Homo sapiens
  misc_feature (1)..(3173): Homo sapiens plasminogen activator, tissue (PLAT), transcript variant 1, mRNA
atggccctgtccactgagcatcctcccgccacacagaaacccgcccagccggggccaccgaccccaccccctgcctggaaacttaaaggaggccggagctgtggggagctcagagctgagatcctacaggagtccagggctggagagaaaacctctgcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggcctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatactgtacttaataaattcagatatatttttcacagtttttccaaaatcagagtggaatggttttgttatagatgctgtatcccactctttattcatgttcacattttaaaatcatttggaattctgcttcactcgcttaacatatacacaacacctgtaacatacaaggcaatgggctaggtgctccagaccgggaaaaggagggacaggaatgcttggtctgatgggctaatatggcatttagagaagtaccaaggtacagtggagccggtcacaaaagggcagacttgtagtagaattcagttgcaagagggattggggaatcttaaggaaaaaatagaatcttaaggaaaaaataactgggtgagacgtggactgtggacaggtgtggaaaaggcactctccatggaggtatgaatatgtagagggccaagagaggggagtacagggagaaatgagttgagcttgtctgaagtgaacttcaggaagaggaacataggctggaatttagattatgggggctctgaacaccaaactgagtttggacttaattgacttctg

SEQ ID NO: 60 - protein, 562 aa, Homo sapiens
  misc_feature (1)..(562): tissue-type plasminogen activator isoform 1 preproprotein
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 61 - DNA, 805 bp, Homo sapiens
  misc_feature (1)..(805): Homo sapiens chemokine (C-C motif) ligand 3-like 1 (CCL3L1), transcript variant 1, mRNA
agaaggacgcaggcagcaaagagtagtcagtcccttcttggctctgctgacactcgagcccacattccatcacctgctcccaatcatgcaggtctccactgctgcccttgccgtcctcctctgcaccatggctctctgcaaccaggtcctctctgcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgcctgaggggtccagaagcttcgaggcccagcgacctcagtgggcccagtggggaggagcaggagcctgagccttgggaacatgcgtgtgacctctacagctacctcttctatggactggttattgccaaacagccacactgtgggactcttcttaacttaaattttaatttatttatactatttagtttttataatttatttttgatttcacagtgtgtttgtgattgtttgctctgagagttccccctgtcccctccaccttccctcacagtgtgtctggtgacaaccgagtggctgtcatcggcctgtgtaggcagtcatggcaccaaagccaccagactgacaaatgtgtatcagatgcttttgttcagggctgtgatcggcctggggaaataataaagatgttcttttaaacggtaaaaaaaaaaaaaaaaaaaaaaaa

SEQ ID NO: 62 - protein, 93 aa, Homo sapiens
  misc_feature (1)..(93): C-C motif chemokine 3-like 1 precursor
MQVSTAALAVLLCTMALCNQVLSAPLAADTPTACCFSYTSRQIPQNFIADYFETSSQCSKPSVIFLTKRGRQVCADPSEEWVQKYVSDLELSA

SEQ ID NO: 63 - DNA, 475 bp, Homo sapiens
  misc_feature (1)..(475): Homo sapiens chemokine (C-C motif) ligand 27 (CCL27), mRNA
cccagataaaaggtaggggaggaggagagagagagaaggaagagtctaggctgagcaacatgaaggggcccccaaccttctgcagcctcctgctgctgtcattgctcctgagcccagaccctacagcagcattcctactgccacccagcactgcctgctgtactcagctctaccgaaagccactctcagacaagctactgaggaaggtcatccaggtggaactgcaggaggctgacggggactgtcacctccaggctttcgtgcttcacctggctcaacgcagcatctgcatccacccccagaaccccagcctgtcacagtggtttgagcaccaagagagaaagctccatgggactctgcccaagctgaattttgggatgctaaggaaaatgggctgaagcccccaatagccaaataataaagcagcattggataataatttctgagtataatctccaaaaaaaaaaaaaaaaaa

SEQ ID NO: 64 - protein, 112 aa, Homo sapiens
  misc_feature (1)..(112): C-C motif chemokine 27 precursor
MKGPPTFCSLLLLSLLLSPDPTAAFLLPPSTACCTQLYRKPLSDKLLRKVIQVELQEADGDCHLQAFVLHLAQRSICIHPQNPSLSQWFEHQERKLHGTLPKLNFGMLRKMG

SEQ ID NO: 65 - DNA, 3353 bp, Homo sapiens
  misc_feature (1)..(3353): Homo sapiens adhesion G protein-coupled receptor E5 (ADGRE5), transcript variant 3, mRNA
caccttcccctcagagcagccagccccaacacagaggccaaagggttcgtcagagccacatggtggaaactctagcagagggtttttgaaagcaggttgcacttcattcttactgagtgcacgtgtgtgtgtgtgtgtgtgtgtgtgtgtgtgtgtgtgtgtatgcagcgcccctgggtctgtgtttttattgcactttcctgctggcttccagctgcaccgccagttccggggagggccctgggccagcggctgtccgccccccctccttcataaagtcctggcctcgggacagcctgcacagctgcctagcctgtggagacgggacagccctgtcccactcactctttcccctgccgctcctgccggcagctccaaccatgggaggccgcgtctttctcgcattctgtgtctggctgactctgccgggagctgaaacccaggactccaggggctgtgcccggtggtgccctcagaactcctcgtgtgtcaatgccaccgcctgtcgctgcaatccagggttcagctctttttctgagatcatcaccaccccgacggagacttgtgacgacatcaacgagtgtgcaacaccgtcgaaagtgtcatgcggaaaattctcggactgctggaacacagaggggagctacgactgcgtgtgcagcccgggatatgagcctgtttctggggcaaaaacattcaagaatgagagcgagaacacctgtcaagatgtggacgaatgtcagcagaacccaaggctctgtaaaagctacggcacctgcgtcaacacccttggcagctatacctgccagtgcctgcctggcttcaagttcatacctgaggatccgaaggtctgcacagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactccaagacaagctcagccgaggtcaccatccagaatgtcatcaaattggtggatgaactgatggaagctcctggagacgtagaggccctggcgccacctgtccggcacctcatagccacccagctgctctcaaaccttgaagatatcatgaggatcctggccaagagcctgcctaaaggccccttcacctacatttccccttcgaacacagagctgaccctgatgatccaggagcggggggacaagaacgtcactatgggtcagagcagcgcacgcatgaagctgaattgggctgtggcagctggagccgaggatccaggccccgccgtggcgggcatcctctccatccagaacatgacgacattgctggccaatgcctccttgaacctgcattccaagaagcaagccgaactggaggagatatatgaaagcagcatccgtggtgtccaactcagacgcctctctgccgtcaactccatctttctgagccacaacaacaccaaggaactcaactcccccatccttttcgccttctcccaccttgagtcctccgatggggaggcgggaagagaccctcctgccaaggacgtgatgcctgggccacggcaggagctgctctgtgccttctggaagagtgacagcgacaggggagggcactgggccaccgagggctgccaggtgctgggcagcaagaacggcagcaccacctgccaatgcagccacctgagcagctttgcgatccttatggctcattatgacgtggaggactggaagctgaccctgatcaccagggtgggactggcgctgtcactcttctgcctgctgctgtgcatcctcactttcctgctggtgcggcccatccagggctcgcgcaccaccatacacctgcacctctgcatctgcctcttcgtgggctccaccatcttcctggccggcatcgagaacgaaggcggccaggtggggctgcgctgccgcctggtggccgggctgctgcactactgtttcctggccgccttctgctggatgagcctcgaaggcctggagctctactttcttgtggtgcgcgtgttccaaggccagggcctgagtacgcgctggctctgcctgatcggctatggcgtgcccctgctcatcgtgggcgtctcggctgccatctacagcaagggctacggccgccccagatactgctggttggactttgagcagggcttcctctggagcttcttgggacctgtgaccttcatcattttgtgcaatgctgtcattttcgtgactaccgtctggaagctcactcagaagttttctgaaatcaatccagacatgaagaaattaaagaaggcgagggcgctgaccatcacggccatcgcgcagctcttcctgttgggctgcacctgggtctttggcctgttcatcttcgacgatcggagcttggtgctgacctatgtgtttaccatcctcaactgcctgcagggcgccttcctctacctgctgcactgcctgctcaacaagaaggttcgggaagaataccggaagtgggcctgcctagttgctggggggagcaagtactcagaattcacctccaccacgtctggcactggccacaatcagacccgggccctcagggcatcagagtccggcatatgaaggcgcatggttctggacggcccagcagctcctgtggccacagcagctttgtacacgaagaccatccatcctcccttcgtccaccactctactccctccaccctccctccctgatcccgtgtgccaccaggagggagtggcagctatagtctggcaccaaagtccaggacacccagtggggtggagtcggagccactggtcctgctgctggctgcctctctgctccaccttgtgacccagggtggggacaggggctggcccagggctgcaatgcagcatgttgccctggcacctgtggccagtactcgggacagactaagggcgcttgtcccatcctggacttttcctctcatgtctttgctgcagaactgaagagactaggcgctggggctcagcttccctcttaagctaagactgatgtcagaggccccatggcgaggccccttggggccactgcctgaggctcacggtacagaggcctgccctgcctggccgggcaggaggttctcactgttgtgaaggttgtagacgttgtgtaatgtgtttttatctgttaaaatttttcagtgttgacacttaaaattaaacacatgcatacagaagaaaaaaaaa

SEQ ID NO: 66 - protein, 786 aa, Homo sapiens
  misc_feature (1)..(786): CD97 antigen isoform 3 preproprotein
MGGRVFLAFCVWLTLPGAETQDSRGCARWCPQNSSCVNATACRCNPGFSSFSEIITTPTETCDDINECATPSKVSCGKFSDCWNTEGSYDCVCSPGYEPVSGAKTFKNESENTCQDVDECQQNPRLCKSYGTCVNTLGSYTCQCLPGFKFIPEDPKVCTDVDECSSGQHQCDSSTVCFNTVGSYSCRCRPGWKPRHGIPNNQKDTVCEDMTFSTWTPPPGVHSQTLSRFFDKVQDLGRDSKTSSAEVTIQNVIKLVDELMEAPGDVEALAPPVRHLIATQLLSNLEDIMRILAKSLPKGPFTYISPSNTELTLMIQERGDKNVTMGQSSARMKLNWAVAAGAEDPGPAVAGILSIQNMTTLLANASLNLHSKKQAELEEIYESSIRGVQLRRLSAVNSIFLSHNNTKELNSPILFAFSHLESSDGEAGRDPPAKDVMPGPRQELLCAFWKSDSDRGGHWATEGCQVLGSKNGSTTCQCSHLSSFAILMAHYDVEDWKLTLITRVGLALSLFCLLLCILTFLLVRPIQGSRTTIHLHLCICLFVGSTIFLAGIENEGGQVGLRCRLVAGLLHYCFLAAFCWMSLEGLELYFLVVRVFQGQGLSTRWLCLIGYGVPLLIVGVSAAIYSKGYGRPRYCWLDFEQGFLWSFLGPVTFIILCNAVIFVTTVWKLTQKFSEINPDMKKLKKARALTITAIAQLFLLGCTWVFGLFIFDDRSLVLTYVFTILNCLQGAFLYLLHCLLNKKVREEYRKWACLVAGGSKYSEFTSTTSGTGHNQTRALRASESGI

SEQ ID NO: 67 - DNA, 1997 bp, Homo sapiens
  misc_feature (1)..(1997): Homo sapiens afamin (AFM), mRNA
actttcttttgtaaatgtggtttctacaaagatgaaactactaaaacttacaggttttatttttttcttgttttttttgactgaatccctaaccctgcccacacaacctcgggatatagagaacttcaatagtactcaaaaatttatagaagataatattgaatacatcaccatcattgcatttgctcagtatgttcaggaagcaacctttgaagaaatggaaaagctggtgaaagacatggtagaatacaaagacagatgtatggctgacaagacgctcccagagtgttcaaaattacctaataatgttttacaggaaaaaatatgtgctatggaggggctgccacaaaagcataatttctcacactgctgcagtaaggttgatgctcaaagaagactctgtttcttctataacaagaaatctgatgtgggatttctgcctcctttccctaccctggatcccgaagagaaatgccaggcttatgaaagtaacagagaatcccttttaaatcactttttatatgaagttgccagaaggaacccatttgtcttcgcccctacacttctaactgttgctgttcattttgaggaggtggccaaatcatgttgtgaagaacaaaacaaagtcaactgccttcaaacaagggcaatacctgtcacacaatatttaaaagcattttcttcttatcaaaaacatgtctgtggggcacttttgaaatttggaaccaaagttgtacactttatatatattgcgatactcagtcaaaaattccccaagattgaatttaaggagcttatttctcttgtagaagatgtttcttccaactatgatggatgctgtgaaggggatgttgtgcagtgcatccgtgacacgagcaaggttatgaaccatatttgttcaaaacaagattctatctccagcaaaatcaaagagtgctgtgaaaagaaaataccagagcgcggccagtgcataattaactcaaacaaagatgatagaccaaaggatttatctctaagagaaggaaaatttactgacagtgaaaatgtgtgtcaagaacgagatgctgacccagacaccttctttgcgaagtttacttttgaatactcaaggagacatccagacctgtctataccagagcttttaagaattgttcaaatatacaaagatctcctgagaaattgctgcaacacagaaaaccctccaggttgttaccgttacgcggaagacaaattcaatgagacaactgagaaaagcctcaagatggtacaacaagaatgtaaacatttccagaatttggggaaggatggtttgaaataccattacctcatcaggctcacgaagatagctccccaactctccactgaagaactggtgtctcttggcgagaaaatggtgacagctttcactacttgctgtacgctaagtgaagagtttgcctgtgttgataatttggcagatttagtttttggagagttatgtggagtaaatgaaaatcgaactatcaaccctgctgtggaccactgctgtaaaacaaactttgccttcagaaggccctgctttgagagtttgaaagctgataaaacatatgtgcctccacctttctctcaagatttatttacctttcacgcagacatgtgtcaatctcagaatgaggagcttcagaggaagacagacaggtttcttgtcaacttagtgaagctgaagcatgaactcacagatgaagagctgcagtctttgtttacaaatttcgcaaatgtagtggataagtgctgcaaagcagagagtcctgaagtctgctttaatgaagagagtccaaaaattggcaactgaagccagctgctggagatatgtaaagaaaaaagcaccaaagggaaggcttcctatctgtgtggtgatgaatcgcatttcctgagaacaaaataaaaggatttttctgtaactgtcacctgaaataatacattgcagcaagcaataaacacaacattttgtaaagtta

SEQ ID NO: 68 - protein, 599 aa, Homo sapiens
  misc_feature (1)..(599): afamin precursor
MKLLKLTGFIFFLFFLTESLTLPTQPRDIENFNSTQKFIEDNIEYITIIAFAQYVQEATFEEMEKLVKDMVEYKDRCMADKTLPECSKLPNNVLQEKICAMEGLPQKHNFSHCCSKVDAQRRLCFFYNKKSDVGFLPPFPTLDPEEKCQAYESNRESLLNHFLYEVARRNPFVFAPTLLTVAVHFEEVAKSCCEEQNKVNCLQTRAIPVTQYLKAFSSYQKHVCGALLKFGTKVVHFIYIAILSQKFPKIEFKELISLVEDVSSNYDGCCEGDVVQCIRDTSKVMNHICSKQDSISSKIKECCEKKIPERGQCIINSNKDDRPKDLSLREGKFTDSENVCQERDADPDTFFAKFTFEYSRRHPDLSIPELLRIVQIYKDLLRNCCNTENPPGCYRYAEDKFNETTEKSLKMVQQECKHFQNLGKDGLKYHYLIRLTKIAPQLSTEELVSLGEKMVTAFTTCCTLSEEFACVDNLADLVFGELCGVNENRTINPAVDHCCKTNFAFRRPCFESLKADKTYVPPPFSQDLFTFHADMCQSQNEELQRKTDRFLVNLVKLKHELTDEELQSLFTNFANVVDKCCKAESPEVCFNEESPKIGN

SEQ ID NO: 69 - DNA, 1924 bp, Homo sapiens
  misc_feature (1)..(1924): Homo sapiens reticulon 4 receptor (RTN4R), mRNA
ctgtgcgccctgcgcgccctgcgcacccgcggcccgagcccagccagagccgggcggagcggagcgcgccgagcctcgtcccgcggccgggccggggccgggccgtagcggcggcgcctggatgcggacccggccgcggggagacgggcgcccgccccgaaacgactttcagtccccgacgcgccccgcccaacccctacgatgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctgacccccagcggacacaagagcgtgctcagcagccaggtgtgtgtacatacggggtctctctccacgccgccaagccagccgggcggccgacccgtggggcaggccaggccaggtcctccctgatggacgcctgccgcccgccacccccatctccaccccatcatgtttacagggttcggcggcagcgtttgttccagaacgccgcctcccacccagatcgcggtatatagagatatgcattttattttacttgtgtaaaaatatcggacgacgtggaataaagagctcttttcttaaaaaaa

SEQ ID NO: 70 - protein, 473 aa, Homo sapiens
  misc_feature (1)..(473): reticulon-4 receptor precursor
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 71 - DNA, 924 bp, Homo sapiens
  misc_feature (1)..(924): Homo sapiens granulysin (GNLY), transcript variant 1, mRNA
gtatctgtggtaaacccagtgacacgggggagatgacatacaaaaagggcaggacctgagaaagattaagctgcaggctccctgcccataaaacagggtgtgaaaggcatctcagcggctgccccaccatggctacctgggccctcctgctccttgcagccatgctcctgggcaacccaggccttgaggtcagtgtgagccccaagggcaagaacacttctggaagggagagtggatttggctgggccatctggatggaaggtctggtcttctctcgtctgagccctgagtactacgacctggcaagagcccacctgcgtgatgaggagaaatcctgcccgtgcctggcccaggagggcccccagggtgacctgttgaccaaaacacaggagctgggccgtgactacaggacctgtctgacgatagtccaaaaactgaagaagatggtggataagcccacccagagaagtgtttccaatgctgcgacccgggtgtgtaggacggggaggtcacgatggcgcgacgtctgcagaaatttcatgaggaggtatcagtctagagttacccagggcctcgtggccggagaaactgcccagcagatctgtgaggacctcaggttgtgtataccttctacaggtcccctctgagccctctcaccttgtcctgtggaagaagcacaggctcctgtcctcagatcccgggaacctcagcaacctctgccggctcctcgcttcctcgatccagaatccactctccagtctccctcccctgactccctctgctgtcctcccctctcacgagaataaagtgtcaagcaagattttagccgcagctgcttcttctttggtggatttgaggggtgggtgtcagtggcatgctggggtgagctgtgtagtccttcaataaatgtctgtcgtgtgtccc

SEQ ID NO: 72 - protein, 172 aa, Homo sapiens
  misc_feature (1)..(172): granulysin isoform 1 precursor
MATWALLLLAAMLLGNPGLEVSVSPKGKNTSGRESGFGWAIWMEGLVFSRLSPEYYDLARAHLRDEEKSCPCLAQEGPQGDLLTKTQELGRDYRTCLTIVQKLKKMVDKPTQRSVSNAATRVCRTGRSRWRDVCRNFMRRYQSRVTQGLVAGETAQQICEDLRLCIPSTGPL

SEQ ID NO: 73 - DNA, 743 bp, Homo sapiens
  misc_feature (1)..(743): Homo sapiens prefoldin subunit 5 (PFDN5), transcript variant 1, mRNA
gaggatcatagagctgtctggcgcagcgaggcctcccggcgccaccgagacgcgcagaggacggctagagcgttgctcgccgagagacttcctcttcgttaagtcggccttcccaacatggcgcagtctattaacatcacggagctgaatctgccgcagctagaaatgctcaagaaccagctggaccaggaagtggagttcttgtccacgtccattgctcagctcaaagtggtacagaccaagtatgtggaagccaaggactgtctgaacgtgctgaacaagagcaacgaggggaaagaattactcgtcccactgacgagttctatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctgagagtttttgcagaaatggggcagagggacaccctttgggcgtggcttcctggtgatgggaagggtcttgtgttttaatgccaataaatgtgccagctgggcagaaaaaaaaaaaaaaaaaaaaaaaaaaaaaaaaaaaaaaaaaaaaaaaaaaaaaaaaaa

SEQ ID NO: 74 - protein, 154 aa, Homo sapiens
  misc_feature (1)..(154): prefoldin subunit 5 isoform alpha
MAQSINITELNLPQLEMLKNQLDQEVEFLSTSIAQLKVVQTKYVEAKDCLNVLNKSNEGKELLVPLTSSMYVPGKLHDVEHVLIDVGTGYYVEKTAEDAKDFFKRKIDFLTKQMEKIQPALQEKHAMKQAVMEMMSQKIQQLTALGAAQATAKA

SEQ ID NO: 75 - DNA, 1078 bp, Homo sapiens
  misc_feature (1)..(1078): Homo sapiens myoglobin (MB), transcript variant 1, mRNA
gcagcctcaaaccccagctgttggggccaggacacccagtgagcccatacttgctctttttgtcttcttcagactgcgccatggggctcagcgacggggaatggcagttggtgctgaacgtctgggggaaggtggaggctgacatcccaggccatgggcaggaagtcctcatcaggctctttaagggtcacccagagactctggagaagtttgacaagttcaagcacctgaagtcagaggacgagatgaaggcgtctgaggacttaaagaagcatggtgccaccgtgctcaccgccctgggtggcatccttaagaagaaggggcatcatgaggcagagattaagcccctggcacagtcgcatgccaccaagcacaagatccccgtgaagtacctggagttcatctcggaatgcatcatccaggttctgcagagcaagcatcccggggactttggtgctgatgcccagggggccatgaacaaggccctggagctgttccggaaggacatggcctccaactacaaggagctgggcttccagggctaggcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaataaaacaacctgcaacatctca

SEQ ID NO: 76 - protein, 154 aa, Homo sapiens
  misc_feature (1)..(154): myoglobin
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 77 - DNA, 2966 bp, Homo sapiens
  misc_feature (1)..(2966): Homo sapiens glypican 5 (GPC5), mRNA
acatcccaggttagctgctgcgagccgagccgggcggcggaggcggcggcggcggcggcagtggcggcagtggcggcagtggcggcagcggcagcagttgcagcagtggtggccagagcggatgcttgcgggctccctgcggctccactagttttcttcgccccgcccagccgcccactcttctcggctagggaagaagaccagagggtgctcagctggaaaactctggtgtctcagcttagggcctcctccgggaagagctaactgctcccaggtgaagccggtgcccgcgggcggtccgtacaccccgcagccggctcgcaccgctcgagagcctcggccgctgtgtcttccacgtctgcagctcagccagggcgcgcagggcgagtggggtccactggcgggtaaaggggaccaggacggcgaggatggacgcacagacctggcccgtgggctttcgctgcctcctccttctggccctggttgggtccgcccgcagcgagggcgtgcagacctgcgaagaagttcggaaacttttccagtggcggctgctgggagctgtcagggggctgccggattcgccgcgggcaggacctgatcttcaggtttgcatatccaaaaagcctacatgttgcaccaggaagatggaggagagatatcagattgcggctcgccaggatatgcagcagtttcttcaaacgtccagctctacattaaagtttctaatatctcgaaatgcggctgcttttcaagaaacccttgaaactctcatcaaacaagcagaaaattacaccagtatacttttttgcagtacctacaggaacatggccttggaggctgctgcttcggttcaggagttcttcactgatgtggggctgtatttatttggtgcggatgttaatcctgaagaatttgtaaacagattttttgacagtctttttcctctggtctacaaccacctcattaaccctggtgtgactgacagttccctggaatactcagaatgcatccggatggctcgccgggatgtgagtccatttggtaatattccccaaagagtaatgggacagatggggaggtccctgctgcccagccgcacttttctgcaggcactcaatctgggcattgaagtcatcaacaccacagactatctgcacttctccaaagagtgcagcagagccctcctgaagatgcaatactgcccgcactgccaaggcctggcgctcactaagccttgtatgggatactgcctcaatgtcatgcgaggctgcctggcgcacatggcggagcttaatccacactggcatgcatatatccggtcgttggaagaactctcggatgcaatgcatggaacatacgacattggacacgtgctgctgaactttcacttgcttgttaatgatgctgtgttacaggctcacctcaatggacaaaaattattggaacaggtaaataggatttgtggccgccctgtaagaacacccacacaaagcccccgttgttcttttgatcagagcaaagagaagcatggaatgaagaccaccacaaggaacagtgaagagacgcttgccaacagaagaaaagaatttatcaacagccttcgactgtacaggtcattctatggaggtctagctgatcagctttgtgctaatgaattagctgctgcagatggacttccctgctggaatggagaagatatagtaaaaagttatactcagcgtgtggttggaaatggaatcaaagcccagtctggaaatcctgaagtcaaagtcaaaggaattgatcctgtgataaatcagattattgataaactgaagcatgttgttcagttgttacagggtagatcacccaaacctgacaagtgggaacttcttcagctgggcagtggtggaggcatggttgaacaagtcagtggggactgtgatgatgaagatggttgcgggggatcaggaagtggagaagtcaagaggacactgaagatcacagactggatgccagatgatatgaacttcagtgatgtaaagcaaatccatcaaacagacactggcagtactttagacacaacaggagcaggatgtgcagtggcgactgaatctatgacattcactctgataagtgtggtgatgttacttcccgggatttggtaactgaactcttctgtcctgacataccttactgaagtctcgatttcttctctctctgcatatgcctggaataagagatcctttttcaatgtaacaattatatttatgaaaagatatgttacactaacttctcagaagccaagctgaaatattcataaagtccctaaaactcaacgtttaaatgacacactttaaaaatatgtcttttttcaatctaactgaaaaccttcttaacttctaatatattaaatctgaagatgtgaagggcacagaagtgactttgaataagaagaatttagtgtatctgtaattttattatcaattccaagccccttcctttctaaattaaaaatgttttcatttgaaagtgtatttgccagacaatgaaaacagtatgcagtatttcttaaagtattgaaattagaatatcatgaaataaatcaaaacatacaatggcaagtagtatgcatgcatattcaagagactcttccatttttgcaagctgtagaaggaaatgtctgaatgtctataagttatggggtagattcttgagaagcatttcatataatttcactgaagaaccttgataattttgacccactgtaacttagccactgatgaaccttaaagctgagtattttattaacacctgatttgtattccattatattcaaaatgcatctttggtattgtgcctctgctcccatctctctctttgcctcatagatttagctatgttgggaagcacatgcttgctctaggaatatctccaataaagctgttaactatttggtggaaaaaaaaaaaaaaaaaaaaaaaaaa

SEQ ID NO: 78 - protein, 572 aa, Homo sapiens
  misc_feature (1)..(572): glypican-5 precursor
MDAQTWPVGFRCLLLLALVGSARSEGVQTCEEVRKLFQWRLLGAVRGLPDSPRAGPDLQVCISKKPTCCTRKMEERYQIAARQDMQQFLQTSSSTLKFLISRNAAAFQETLETLIKQAENYTSILFCSTYRNMALEAAASVQEFFTDVGLYLFGADVNPEEFVNRFFDSLFPLVYNHLINPGVTDSSLEYSECIRMARRDVSPFGNIPQRVMGQMGRSLLPSRTFLQALNLGIEVINTTDYLHFSKECSRALLKMQYCPHCQGLALTKPCMGYCLNVMRGCLAHMAELNPHWHAYIRSLEELSDAMHGTYDIGHVLLNFHLLVNDAVLQAHLNGQKLLEQVNRICGRPVRTPTQSPRCSFDQSKEKHGMKTTTRNSEETLANRRKEFINSLRLYRSFYGGLADQLCANELAAADGLPCWNGEDIVKSYTQRVVGNGIKAQSGNPEVKVKGIDPVINQIIDKLKHVVQLLQGRSPKPDKWELLQLGSGGGMVEQVSGDCDDEDGCGGSGSGEVKRTLKITDWMPDDMNFSDVKQIHQTDTGSTLDTTGAGCAVATESMTFTLISVVMLLPGIW

SEQ ID NO: 79 - DNA, 6076 bp, Homo sapiens
  misc_feature (1)..(6076): Homo sapiens arylsulfatase B (ARSB), transcript variant 1, mRNA
aaaagtgaatacatgattttatttaactcattaataaggaaattggtaaggtgttaaaaccaattcaaaggacaatccaaagaacagatcaggaatactaaaataaatatgcaagcggaggtgaaactgttttccttggtagtggtggaggggaaggattgctactccgctggataaagttcatttgtgtatatataaataagaattattttccattgttatttatctataacttataaagttgtaaacaacttccacggaatcagactcaacctggaagggtatggtctctaggcaatgcaaaaattttcccctacacctgttaacaactataatatctccagacagagtagacagaaagtctggatggcaacgggaatctactggtcatacggctaacttcctaattcaataagcacgtgactaaaggattttttccttccactcagatatttcaggctaactagatactgtgtgcttcttagtgtcactgcttagtgggggagccagctctgagtggggtcatatccggacaagcgaatgagctatttattcaatgaccacgcaacactccaaatcctcccagggcaacttgaaagtaaccgcaccttccaaagggcaccgtgcaatcagactgtgtgtttggcctcctgtttgctagtggggaggaagcggcttcatgggtgtacactacgcataaatgaatgtgaaaggctatttagacctctgccttttcaccgtcctcccacctgccacaggctgggctcttgtgctagaaatgacttgctagctagacatcatggttcaggatctgagtcagaggtttaaccatttataagcttttttcttatgaaaaattggcactaattataatgtctaactgtcagagttgttgcaggctttacaggagacgcgggctgtgaagatgctttgtaaattgtgaagcgttattaaagaacacatcttttttttttaggaaaccacagtgcaaatttaattgccggggaagataacgggccttggtgccctccaagcgtcagctgagtttccaagaagccgggcagcgggcgcccgcgggttcgtctctggctcctcctccgccacagcagccgggggcccgggtcggaggcggcgggggccgagcgcccggcctcgcaagcccacggcccgctgggggtgccgtcccgcgccggggcggagcaggccccggcagcccagttcctcattctatcagcggtacaaggggctggtggcgccacaggcgctgggaccgcgggcggacaaggatgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctcatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctccagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtaggatttcagggaggctagaaaacctttcaattggaagttggacctcaggccttttctcacgactcttgtctcatttgttatcccaacctgggttcacttggcccttctcttgctcttaaaccacaccgaggtgtctaatttcaacccctaatgcatttaagaagctgataaaatctgcaacactcctgctgttggctggagcatgtgtctagaggtgggggtggctgggtttatccccctttcctaagccttgggacagctgggaacttaacttgaaataggaagttctcactgaatcctggaggctggaacagctggctcttttagactcacaagtcagacgttcgattcccctctgccaatagccagttttattggagtgaatcacatttcttacgcaaatgaagggagcagacagtgattaatggttctgttggccaaggcttctccctgtcggtgaaggatcatgttcaggcactccaagtgaaccacccctcttggttcaccccttactcacttatctcatcacagagcataaggcccattttgttgttcaggtcaacagcaaaatgcctgcaccatgactgtggcttttaaaataaagaaatgtgtttttatcgtaatttatttccccccagccattgctcactctgtctagacttcctgccacttccaattcttctgtggcttttcctgcctttccttttgacctcagtagtcctatccctgggaaggccactttgcttctctacctgagcacccctgatttctggaacgctgctgagccctgccttacttttgcccctagggctgaagctagaggcctccccgtaataggcggtggagttgctctgtgaggatgttcatggtagacactaagagggctgggtgggagatgcttggctctgtggcatctgttcagcgaggcttttcctatattgcatggagttagtcattgtgattgtagctttatttcataatatattaagacttgcactgctatttactagcagtgagaagaaacctcaggaaaggatatgaaaaagcaagtggccagtgtctgggatactgggccttggtaaagcagaggagggcacacccacagtcctcttattctctgttttactgcttgttttgaggttctggggtctggcaaagaggatgcagtttgacacctgcagccctttctcaatcccactaatgtcttactaatgtggaacagtccatattagctccagagagtgtcaaacccagagaaatgtgtgcaaaaatgatactcttttctgcattagccccaccattgtgttcaccaatgcttggaacactgcctgaaggcactcattttttaatttttattttatttttaattttttatatctttatgagacgatctcactctgtcaccaggttggagtacagtggtacaatcacaactcaccgtagcctcaaactcctgggctcaagtgattctcccacctcaggcacccaaatagctggaactacaggcatataccgccacacccagctaattttattttttgaaaagacaaggttccctatgttgcccagctggtcttaaactcctgggctccagcaattatcccagcttgggctccaaaagtgctgggattacaggcatgagtcaccatgcctggcctcattttttaaaacaaatgaataaatggacaaatgagtaaatgagaaagtctcacaccatgaaagatgctagtccaatgagctgaatacagaggtaatataaatgtcttccagctgttgcttttctgttctcaagctgcccctcctggggtaggagcataatctacatcactgggcagtcacaggacactctatagcaaggttgtagcgtcctctccagtggggggagaaaaggaactgtgcctaccaaaggtactctcttgtcagcaatttccatttctatactttatgggacactagaaactaaaagcaacaaataatctgatataagtccttgtatagtcatccttcaattcagtagcaatattttctggtcactactaacctgtattgtattaaaatgagactattggaaggaaatggtgctaaaactaataacatctcttaccaacctttacccaactcctgggttggcaaacagctgaccaaactgccatcacctcccacttggaagtgtatggccgacagcatgaaatagctgagcccagatgttccttctgcatcctccgaatcccagggctgggtgtaggtagccgttggaggccatcgctacagggcacctatctgttatcgctgctgtcctcccaacagctgtctccagttctagttccttggttttcaggcacagtgggggatgttctgcacccagtggacttcaaaagagttttgaagacttaattttttgtaaaacaagtacttgagattttggtttatccataatagaatgtatttcattagattctctgattctatataagaatgtgaaaagattgatatattgttgttagaaataatgttatttctttccaattttttttttttttttttttgagatggagtctcgctctgtcacccaggctggagtgcagtggtgtgatctcggctcactgcagcctctaactcccaggttcaagctattctcctgcctcagcctcccaagtagctggattacaggcatacaccaccacgcctggctatgttttgtatttttcgtagagatagggtttcaccatgttggccaggctggtctcaaactcctgacctcaagtgatccacccacttcagcttcccaaagcactgggattacaggtgtgagccactgtgcccggcaaatttttttacctttacagaaggttttgcttatttaattgtgagctcatttttctttgttacttttgtccccccagatttgggggacaaaataaaattaatcttttaaaatgtgtcagccatatgtatggggcttccatttggggtgaggagaaagttctggaactagatagtggtcatggttatacaacatcataaatgcaattactgccactgaattgtatgttttaaagtggttaaaatgttaagttttatgttttattacaatttttaaatgtgtcaaccaactttatagtacataaattatatctcagtaaagctgttaaataaataaatatagtaaaaattttagaactaaaaaaa

SEQ ID NO: 80 - protein, 533 aa, Homo sapiens
  misc_feature (1)..(533): arylsulfatase B isoform 1 precursor
MGPRGAASLPRGPGPRRLLLPVVLPLLLLLLLAPPGSGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 81 - DNA, 6061 bp, Homo sapiens
  misc_feature (1)..(6061): Homo sapiens sortilin-related VPS10 domain containing receptor 2 (SORCS2), mRNA
atggcgcaccgggggccctcgcgcgcctcgaagggccccggccccaccgcccgagccccgagccccggggctccgccgccgccgcgctcgccgcgctcgcggccgctcctgctgctgctgctgctgctgggcgcctgcggggcggcggggcgctcccctgagcccgggcgcctgggtcctcacgcccaactgacccgggtgccgcggagccctcccgcggggcgcgcggagcccggtggcggcgaggaccggcaggcgcgcggcacggagccaggcgccccgggtccgagtcccggtcccgctcctggtcccggcgaggacggcgcccccgccgcgggctaccggcgctgggagcgggcggcgccgctggccggagtggcttcgcgggcgcaggtctcgctcatcagcacgtcgttcgtgctcaagggggacgcgacgcacaaccaggcgatggtgcactggacgggcgagaacagcagcgtgatcttgatcctgacgaagtactaccacgcagacatggggaaggttctggaaagttctctgtggcggtcatcagatttcgggacgtcctacaccaagctcaccctccagcctggtgtcaccaccgtcatcgacaatttctacatctgcccgaccaacaagaggaaggtcatccttgtcagctcctcactcagtgaccgggaccagagcctattcctcagcgcagacgaaggcgccacctttcagaagcagcccattcccttcttcgtggaaactctgattttccaccctaaggaggaggacaaggtcctcgcctacacaaaggagagcaagctctacgtgtcatctgacttggggaaaaagtggacacttctgcaagagcgagtgaccaaagaccacgtgttctggtctgtgtctggggtggacgctgaccctgacttggtccacgtggaagcccaagacctcggtggagattttcggtacgtcacctgcgcaatccacaattgctccgagaagatgctgacagccccattcgcaggccccattgaccacgggtctctgaccgtgcaggacgattacatcttctttaaggcaacatcagcaaaccagacaaaatactacgtctcttatcgtcgaaatgaatttgtcctgatgaagctgccgaagtatgcattgccaaaggatctgcagatcatcagcacggacgagagtcaggtgttcgtggcggtgcaggagtggtaccagatggacacctacaacctgtaccagtcggacccacggggcgtgcgctacgcgctggtgctgcaggacgtgcgcagctcacggcaggcggaggagagcgtgctcatcgacatcctggaggtcagaggggtgaaaggagtcttcctggcaaaccaaaaaattgatgggaaagtgatgacgcttataacctacaacaagggccgcgactgggattacctgaggccacccagcatggacatgaatggaaaaccaaccaactgcaagcctccagactgccacctgcacctgcacctgcgctgggcagacaacccctacgtatcaggcaccgtgcacaccaaggacaccgccccaggcctcatcatgggtgcaggtaacctgggctcacagctggtggaatataaagaagaaatgtacatcacgtcagactgtggtcacacctggcggcaggtgtttgaggaagagcatcacatcctgtacctggaccacggcggcgtgatcgtggccatcaaagacacctccatccctttgaagatcctcaagttcagtgtggacgagggcctcacctggagcacgcacaacttcaccagcacctcggtgtttgtggacgggctgctgagtgagccaggggacgagacgctggtcatgacggtctttggccacatcagcttccgctccgattgggagctggtcaaggtggacttccggccctcattctccaggcagtgcggcgaggaggactacagctcctgggagctctccaacctgcagggcgaccgctgtatcatgggccagcagagaagtttccggaaaagaaagtccacgtcctggtgcatcaaggggaggagcttcacgtcggcgctcacgtcccgcgtgtgcgagtgccgggactcggacttcctgtgcgactacggatttgagcgctcctcctcctcagagtccagcaccaacaagtgctctgccaacttctggtttaacccattgtccccgcctgacgactgtgccctgggccagacctacaccagcagccttgggtaccggaaagtggtgtccaacgtgtgtgagggtggggtggacatgcagcagagtcaggtgcagctgcagtgccccctcacgccgccccggggcctgcaggtcagcattcaaggcgaggcggtggccgtgcggcctggagaggacgtcctgtttgtggtgcggcaggagcagggtgatgtcctgactaccaagtaccaggtagaccttggggacggcttcaaggccatgtacgtgaaccttacactgaccggggagcccatccggcaccgctacgagagccccggcatctaccgcgtgtccgtcagggcagagaacacggcaggccacgatgaggcggtgctctttgtccaggtcaactcccccctgcaggccctctacctggaggtggttcctgtcattggcctcaaccaggaggtgaacctcacagctgtgctgcttcccttgaaccctaacctcaccgtcttctactggtggatcggccacagcctgcagcccctcctttccctggataattctgtgacaacgcggttttcggacacgggcgacgtgcgtgtgacggtgcaggccgcctgtgggaactcggtgctgcaggactccagggtcctccgtgtgctggatcaatttcaagtcatgcctctgcagttttccaaggagctggatgcctacaaccccaacacccctgagtggagggaagacgtgggcctggtggtcacccggctgctctccaaggagaccagcgtccctcaggagcttctggtgactgtggtgaagccggggctgcccactttggccgatctgtacgtgctcctgccccctcccaggcccacaaggaagaggagcctctcgagtgataagaggctcgccgccatccagcaggtgctgaacgcacagaagatcagcttcctcctgcgaggcggagtccgggtcctggtggccctgcgggacacaggcacaggtgctgagcagctgggcggcggtggcggctactgggcggtagtggtgctgtttgtcatcgggctcttcgcagcgggagccttcatcctctacaagttcaaaaggaaacggccaggcaggaccgtgtacgcccaaatgcacaacgagaaggagcaggagatgaccagccctgtgagtcacagtgaggacgtccagggcgctgtccagggcaaccactcaggcgtggtcctgagcatcaactcccgagagatgcacagctacctggtgagctgatgccaccccagcatctgtcttttcacccacggagggcacagaaccaccagcaaagccggcggctggactggcgcccctcagagacctgcggaaagccccctccctgagtcgtcgccacaccaggcgacaggcaccaccccctctgataaatccaagcccgcccaggcccacggggggcccacgggaccccccgggactccccggacatggccctgcccctatgggacaccaggcctgactcaggcaggttctgccccccagaccccacacacggccgccccacgtgctgtcgctcagcccgaggcctgacttctctgggctgaggctggtcgtcctggagccctcccagtacctcgggctgcaagagctgcagacccgttcagacactgcgttgcgggctccttccccgcagaggccggggcctccctgactttgcttcttcactcccacctgtgcggccacccagtccctctgtgggagcccgggtgccaggccctcccaacaccacaccaccctccaggcccccctgccctccggctggaaactgcagctctgtaaatgcctctctaggtagctgctggcggtggtgggggtgtctcactctctgtctttatagccggcggtagccaccggggtggctctgtcagagttccgtactcgggagcccctttccctgagtgcccagggtgtctcctctgcccagaggggcagcagctctccctggttctccccagggcagacggggtagggcgggctcaggacccagtgcccatggttcctcactcctaccagcaagcagcaccatcccgcaggcttctcccacctgatggctgttctcccaccgggtctgggctctggaaggagccagatgcccccagaaaggtgggtggtggagacggcaccagatgtaccagttttctgcagttccttataggcgaagggaaccgggtggaaaccaagctgggagaggctgaggatggcagggctggaagggccatcagcgtgaccctcattttcaagaaggggaaactgaggcccagggaggagagtaactgaaggtcacagcacggtcacagcagaggtggccgaacccagccctctgcgcgccagtgctgtgcggtctccacacccttacggtttcctagaatcagggatgttagtgtaagtctataggaatataggggggtgggggggtcaccttttgccttgaaatgggaagtcagtagccccttcctcctcctccctcctccccctcctctctggcagggatctcagatgaccgtggcctccctctcagagggggagaacgccagagccctggctggtgatgtgctggctgggggtgaatcccaatgagggtccctctcagagcgggagaacgccagagccctggatggtgatgcgctggctgggggtgaatcccaatgagggtccctctcagaaagggagaacgccagagccctggctggtgacatgctggctgggggtgaatccgaatgacagtgcagacgttctcccatccaccatgtctgagcttgggggaattgcctcatttcccctggaaaagaaacatggtccattagaggggaaagcccaggggtgaatcttcacgccccaaacagtgcccggtggggaggaggcacccgctccttgttgagtaaaaccacccatggagactggaacctcatctccctgggtcggggggtgttcaaggccacaggacaaggggagcaccctgggccacacaggcgtggaggtgtccccaccccttccacctgtcccccagacccaaagctctctccccaccctacctgcccacctggggctcctgtgccccctccccactccagaggccaccctacaagttgtcctcaaggtcatcctggagatgggatccaggacgtggggccatgactctctgggaccttgccacagcccccattcccctgcttgcagtctgcaaggacacctttgcagggattcttgtcctgctggccaccccacccacacctgtccctggccagcaggccgcctgcaagcgtcaggcacacagggacagacatggcgagcacagtgcaggcccggggcccacgggcaacatggaaccctgggaactgccctcccccttagctcacagtgcctgcggtagccactctaggtcgttggccttccttgaccactccatttaattctctctgctgtttgggttgggtttttccccttagttatctgtgggtttcctgtattttatgttaatatttctattaagaacatgttgggcatgtggacccaagcacctgggaaggaggtggcatctgagacagcctgatacgttcccgtctgtgcacccatggagatccaggcgtgggcccgtgtctgtccctggttgtaaattcgagggtctgcatatctgatgttcaggtagacctgggcgcctgggaacgaggccatcagctgccatgcacataacaaagagacaatgcattccttcttatttttcctttttaaaaatcgatgaatcatttgtgatgcttttaacaaagattaaatgaatttgatcagcttttgccttaaaaaaaaa

SEQ ID NO: 82 - protein, 1159 aa, Homo sapiens
  misc_feature (1)..(1159): VPS10 domain-containing receptor SorCS2 precursor
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTSPVSHSEDVQGAVQGNHSGVVLSINSREMHSYLVS